SEQUENCE LISTING (11 sequences)

SEQ ID NO: 1 - protein, 508 aa, synthetic construct
MEPAGPAPGRLGPLLLCLLLSASCFCTGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAADNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 2 - protein, 293 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 3 - protein, 305 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQRTIQPPRNR

SEQ ID NO: 4 - protein, 312 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQRTIQPPRKAVEEPLNE

SEQ ID NO: 5 - protein, 323 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQRTIQPPRKAVEEPLNAFKESKGMMNDE

SEQ ID NO: 6 - DNA, 22 bp, synthetic construct
  source 1..22: mol_type = other DNA
gatgttgcaacagcagcgagaa

SEQ ID NO: 7 - DNA, 20 bp, synthetic construct
  source 1..20: mol_type = other DNA
caccggactgggctagttca

SEQ ID NO: 8 - DNA, 20 bp, synthetic construct
  source 1..20: mol_type = other DNA
atgcaatactggcccccagg

SEQ ID NO: 9 - DNA, 22 bp, synthetic construct
  source 1..22: mol_type = other DNA
gatttggggctgctggtttcaa

SEQ ID NO: 10 - DNA, 25 bp, synthetic construct
  source 1..25: mol_type = other DNA
tcagacgatttgtaagatgcaggga

SEQ ID NO: 11 - DNA, 23 bp, synthetic construct
  source 1..23: mol_type = other DNA
tgtagcagatcctggccttggac

(19) United States
(12) Patent Application Publication
(10) Pub. No.: US 2024/0102045 A1
(43) Pub. Date: Mar. 28, 2024

(54) VECTORS, GENETICALLY MODIFIED CELLS, AND GENETICALLY MODIFIED NON-HUMAN ANIMALS COMPRISING THE SAME

(71) Applicant: Regeneron Pharmaceuticals, Inc., Tarrytown, NY (US)

(72) Inventors: Davor Frleta, Tarrytown, NY (US); Naxin Tu, Tarrytown, NY (US)

(21) Appl. No.: 18/223,434

(22) Filed: Jul. 18, 2023

Related U.S. Application Data
(60) Provisional application No. 63/390,513, filed on Jul. 19, 2022.

(51) Int. Cl. C12N 15/85; C12N 5/073; G01N 33/50

(52) U.S. Cl. CPC C12N 15/8509; C12N 5/0603; G01N 33/5008

(57) ABSTRACT

Provided herein are genetically modified cells and genetically modified non-human animals (e.g., rats and mice) comprising: (i) a Rag1 and/or Rag2 gene knock-out; (ii) a IL2rg gene knock-out; and (iii) a homozygous null mutation in the non-human animal Heme oxygenase-1 (Hmox-1) gene, and optionally a Fah gene knock-out and/or expressing one or more human or humanized polypeptides. Methods and compositions of making and using such genetically modified cells and non-human animals are also provided.

### RELATED APPLICATIONS

This application claims priority to U.S. Provisional Application No. 63/390,513, filed Jul. 19, 2022, which is hereby incorporated herein by reference in its entirety.

### SEQUENCE LISTING

The instant application contains a Sequence Listing which has been submitted electronically in XML format and is hereby incorporated by reference in its entirety. Said XML copy, created on Nov. 29, 2023, is named RPD-00101_SL.xml and is 12,431 bytes in size.

### BACKGROUND

Genetically modified cells, genetically modified mice comprising the same, as well as modified and engrafted mice, and their use in modeling human diseases, e.g., for the purpose of drug testing, are known in the art. Use of genetically modified mice to model a human immune system (HIS) has been reported (Manz (2007) Immunity, 26:537-541). For example, HIS mice generated by transplanting a severely immunodeficient mouse strain (such as Rag2 KO Il2rg KO mice) with human hematopoietic stem and progenitor cells have been reported. Although multi-lineage hematopoietic development has been observed in these HIS mice, they are known to lack human red blood cells (hRBCs) in the peripheral blood. It was found that human RBC progenitors can develop in the bone marrow of such mice but fail to survive in the periphery. This impeded the use of these HIS models in modeling diseases or infections related to human red blood cells and in testing effects of therapeutic drugs on human red blood cells.

Thus, there is a need for genetically modified mice that can support maintenance and propagation of human red blood cells, and for mice suitable for engraftment that can model or approximate certain aspects of human red blood cells.

### SUMMARY

The present disclosure is based, in part, on the discovery that knocking out Hmox-1 gene in immune-compromised mice (e.g., mice having a Rag1 and/or Rag2 gene knockout and an IL2rg gene knockout) that are engrafted with human hematopoietic cells restored human red blood cells in the peripheral blood.

Accordingly, in one aspect, the present disclosure relates to genetically modified non-human animals (e.g., rodents) comprising (i) a homozygous null mutation in Rag2 gene; (ii) a homozygous null mutation in IL2rg gene; and (iii) a homozygous null mutation in the non-human animal Heme oxygenase-1 (Hmox-1) gene. In some embodiments, the genetically modified non-human animal comprises a homozygous null mutation in Rag1 gene. In some embodiments, the null mutation in Hmox-1 gene is a deletion of at least exons that correspond to mouse Hmox-1 exons 3-5. In some embodiments, the null mutation in Hmox-1 gene is a deletion of the full Hmox-1 endogenous coding sequence.

In some embodiments, the genetically modified non-human animal comprises a homozygous null mutation in Fah gene. In some embodiments, the homozygous null mutation in Fah gene comprises an insertion, a deletion, and/or a substitution in the endogenous Fah gene.

In some embodiments, the genetically modified non-human animal expresses a human or humanized SIRPA protein encoded by a nucleic acid operably linked to a Sirpa promoter. In some embodiments, the genetically modified non-human animal comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter. In some embodiments, the Sirpa gene comprises exons 2-4 of a human SIRPA gene. In some embodiments, the genetically modified non-human animal expresses a Sirpa polypeptide comprising an extracellular portion of a human SIRPA polypeptide and an intracellular portion of a non-human animal Sirpa polypeptide. In some embodiments, the non-human animal Sirpa polypeptide is an endogenous non-human animal Sirpa polypeptide, and/or the non-human animal Sirpa gene is an endogenous non-human animal gene. In some embodiments, the genetically modified non-human animal expresses a human SIRPA polypeptide encoded by a nucleic acid operably linked to a Sirpa promoter.

In some embodiments, the genetically modified non-human animal further expresses one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to an EPO promoter.

In some embodiments, at least one promoter operably linked to a nucleic acid that encodes a human or humanized protein is an endogenous non-human animal promoter. In some embodiments, all promoters operably linked to the nucleic acids that encode the human or humanized proteins are endogenous non-human animal promoters. In some embodiments, the endogenous non-human animal promoter is at the corresponding non-human animal gene locus.

In some embodiments, the genetically modified non-human animals described herein comprise a null mutation in at least one corresponding non-human animal gene at the corresponding non-human animal gene locus.

In some embodiments, the genetically modified non-human animal is heterozygous for at least one allele comprising the nucleic acid sequence that encodes the human or humanized protein. In some embodiments, the genetically modified non-human animal is homozygous for at least one allele comprising the nucleic acid sequence that encodes the human or humanized protein.

In some embodiments, at least one nucleic acid comprises genomic coding and noncoding sequence for the human or humanized protein. In some embodiments, at least one nucleic acid comprises cDNA sequence for the human or humanized protein.

In some embodiments, the genetically modified non-human animal expresses a human M-CSF protein encoded by a nucleic acid operably linked to an M-CSF promoter.

In some embodiments, the genetically modified non-human animal expresses a human or humanized CD47 protein encoded by a nucleic acid operably linked to a CD47 promoter. In some embodiments, the genetically modified non-human animal expresses a humanized CD47 protein, and the humanized CD47 protein comprises an extracellular portion of a human CD47 protein and an intracellular portion of an endogenous non-human animal CD47 protein.

In some embodiments, the genetically modified non-human animal expresses: (i) a human or humanized SIRPA protein encoded by a nucleic acid operably linked to a Sirpa promoter; (ii) a human M-CSF protein encoded by a nucleic acid operably linked to an M-CSF promoter; and (iii) a human or humanized CD47 protein encoded by a nucleic acid operably linked to a CD47 promoter.

In some embodiments, the genetically modified non-human animal expresses a human EPO protein encoded by a nucleic acid operably linked to an EPO promoter.

In certain aspects, the genetically modified non-human animal described herein further comprises an engraftment of human hematopoietic cells. In some embodiments, human hematopoietic cells comprise one or more cells selected from the group consisting of a human CD34-positive cell, a human hematopoietic stem cell, a human hematopoietic progenitor cell, a human erythroid precursor cell, and a human erythrocyte. In some embodiments, the animal comprises human cells of erythroid lineage.

In some embodiments, the non-human animal further comprises an infection with a pathogen that targets human cells of the erythroid lineage. In some embodiments, the animal comprises the inactivated endogenous FAH gene, and further comprises transplanted human hepatocytes. In some embodiments, the pathogen can cause malaria in human. Such pathogen may be selected from a Plasmodium sp., Babesia sp., and a Theileri sp.

In some embodiments, the engrafted human hematopoietic cells give rise to abnormal human cells of the erythroid lineage. In some embodiments, the engrafted human hematopoietic cells comprise a mutation in β-globin gene that leads to sickle cell disease.

In some embodiments, the genetically modified non-human animal is a mammal. In some embodiments, the mammal is a rodent, such as a rat or a mouse. In some embodiments, the rodent is a mouse.

In certain aspects, provided herein is a method for identifying an agent that inhibits an infection by a pathogen that targets human cells of the erythroid lineage, the method comprising: a. administering the agent to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; iii. an engraftment of human hematopoietic cells; and iv. an infection by a pathogen that targets human cells of the erythroid lineage, and b. determining whether the agent reduces the amount of the pathogen and/or inhibits the activity of the pathogen in the pathogen-infected non-human animal.

In certain aspects, provided herein is a method for identifying an agent that prevents an infection by a pathogen that targets human cells of the erythroid lineage, the method comprising: a. administering the agent to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic cells, b. injecting the genetically modified non-human animal with parasitized reticulocytes or erythrocytes, and c. determining whether the agent prevents the infection of the human reticulocytes and/or erythrocytes of the non-human animal. In some embodiments, the pathogen can cause malaria in human. Such pathogen may be selected from a Plasmodium sp., Babesia sp., and a Theileri sp.

In certain aspects, provided herein is a method for identifying an agent that treats sickle cell disease, the method comprising: a. administering the agent to a genetically modified non-human animal, wherein the genetically modified non-human animal comprises: i. a homozygous null mutation in the non-human animal Hmox-1 gene; ii. a homozygous null mutation in Rag2 gene t and a homozygous null mutation in IL2rg gene; and iii. an engraftment of human hematopoietic cells comprising a mutation in β-globin gene that leads to sickle cell disease, and b. determining whether the agent prevents or reduces red cell sickling in the non-human animal.

In certain aspects, provided herein is a method for assessing therapeutic efficacy of a drug candidate targeting human red blood cells, the method comprising: a. administering the drug candidate to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic progenitor cells, and b. monitoring the human red blood cells in the non-human animal to assess the therapeutic efficacy of the drug candidate.

In some embodiments, the human red blood cells are monitored to determine whether number of the human red blood cells in the non-human animal is reduced by the drug candidate. In some embodiments, the drug candidate is a chemotherapeutic agent, or an anti-malaria agent. In some embodiments, the human red blood cells are monitored to assess whether the drug candidate induces agglutination of the red blood cells. In some embodiments, the drug candidate is a modulator (e.g., an antibody) of a human CD47 protein.

In certain aspects, provided herein is a method of identifying an agent that reduces toxicity of a toxic drug on human red blood cells, comprising: a. administering the agent and the toxic drug to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic progenitor cells, and b. determining whether the agent reduces the toxicity of the toxic drug on human red blood cells in the non-human animal. In some embodiments, the agent and the toxic drug are administered to the non-human animal concurrently or sequentially. In some embodiments, the toxicity is on-target toxicity or off-target toxicity.

In certain aspects, provided herein is a genetically modified non-human animal cell, comprising: (i) a homozygous null mutation in Rag2 gene; (ii) a homozygous null mutation in IL2rg gene; and (iii) a homozygous null mutation in the non-human animal Heme oxygenase-1 (Hmox-1) gene.

In some embodiments, the genetically modified non-human animal cell comprises a homozygous null mutation in Rag1 gene. In some embodiments, the null mutation is a deletion of at least exons that correspond to mouse Hmox-1 exons 3-5. In some embodiments, the null mutation is a deletion of the full Hmox-1 endogenous coding sequence.

In some embodiments, the genetically modified non-human animal cell comprises a homozygous null mutation in Fah gene. In some embodiments, the homozygous null mutation in Fah gene comprises an insertion, a deletion, and/or a substitution in the endogenous Fah gene.

In some embodiments, the genetically modified non-human animal cell expresses a human or humanized SIRPA polypeptide encoded by a nucleic acid operably linked to a Sirpa promoter. In some embodiments, the genetically modified non-human animal cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter. In some embodiments, the Sirpa gene comprises exons 2-4 of a human SIRPA gene. In some embodiments, the genetically modified non-human animal cell expresses a Sirpa polypeptide comprising an extracellular portion of a human SIRPA polypeptide and an intracellular portion of a non-human animal Sirpa polypeptide. In some embodiments, the non-human animal Sirpa polypeptide is an endogenous non-human animal Sirpa polypeptide, and/or the non-human animal Sirpa gene is an endogenous non-human animal gene. In some embodiments, the genetically modified non-human animal cell expresses a human SIRPA polypeptide encoded by a nucleic acid operably linked to a Sirpa promoter.

In some embodiments, the genetically modified non-human animal cell further expresses one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to an EPO promoter.

In some embodiments, at least one promoter operably linked to a nucleic acid that encodes a human or humanized protein is an endogenous non-human animal promoter. In some embodiments, all promoters operably linked to the nucleic acids that encode the human or humanized proteins are endogenous non-human animal promoters. In some embodiments, the endogenous non-human animal promoter is at the corresponding non-human animal gene locus.

In some embodiments, the genetically modified non-human animal cell comprises a null mutation in at least one corresponding non-human animal gene at the corresponding non-human animal gene locus. In some embodiments, the genetically modified non-human animal cell is heterozygous for at least one allele comprising the nucleic acid sequence that encodes the human or humanized protein. In some embodiments, the genetically modified non-human animal cell is homozygous for at least one allele comprising the nucleic acid sequence that encodes the human or humanized protein.

In some embodiments, the genetically modified non-human animal cell expresses a human M-CSF protein encoded by a nucleic acid operably linked to an M-CSF promoter.

In some embodiments, the genetically modified non-human animal cell expresses a human or humanized CD47 protein encoded by a nucleic acid operably linked to a CD47 promoter.

In some embodiments, the genetically modified non-human animal cell expresses a humanized CD47 protein, and the humanized CD47 protein comprises an extracellular portion of a human CD47 protein and an intracellular portion of an endogenous non-human animal CD47 protein.

In some embodiments, the genetically modified non-human animal cell expresses: (i) a human or humanized SIRPA protein encoded by a nucleic acid operably linked to a Sirpa promoter; (ii) a human M-CSF protein encoded by a nucleic acid operably linked to an M-CSF promoter; and (iii) a human or humanized CD47 protein encoded by a nucleic acid operably linked to a CD47 promoter.

In some embodiments, the genetically modified non-human animal cell expresses a human EPO protein encoded by a nucleic acid operably linked to an EPO promoter.

In some embodiments, the genetically modified non-human animal cell is a mammalian cell. In some embodiments, the mammalian cell is a rodent cell, such as a rat cell or a mouse cell. In some embodiments, the rodent cell is a mouse cell.

In some embodiments, the genetically modified non-human animal cell is a non-human animal embryonic stem (ES) cell.

In certain aspects, provided herein is a method of making a non-human animal embryonic stem cell, comprising genetically engineering the non-human animal embryonic stem cell so that the non-human animal embryonic stem cell has a genome that comprises: (i) a homozygous null mutation in Rag2 gene; (ii) a homozygous null mutation in IL2rg gene; and (iii) a homozygous null mutation in the non-human animal Heme oxygenase-1 (Hmox-1) gene.

In certain aspects, provided herein is a non-human animal embryo comprises the non-human animal embryonic stem cell described herein, or the non-human animal embryonic stem cell made according to the method described herein.

In certain aspects, provided herein is a method of making a non-human animal comprising in its genome: (i) a homozygous null mutation in Rag2 gene; (ii) a homozygous null mutation in IL2rg gene; and (iii) a homozygous null mutation in the non-human animal Heme oxygenase-1 (Hmox-1) gene, the method comprising steps of: (a) obtaining a non-human animal embryonic stem cell described herein, or the non-human animal embryonic stem cell made according to the method described herein; and (b) creating a non-human animal using the non-human animal embryonic cell of (a).

In certain aspects, provided herein is a method of making a non-human animal comprising in its genome: (i) a homozygous null mutation in Rag2 gene; (ii) a homozygous null mutation in IL2rg gene; and (iii) a homozygous null mutation in the non-human animal Heme oxygenase-1 (Hmox-1) gene, the method comprising modifying the genome of the non-human animal so that it comprises: (i) a homozygous null mutation in Rag2 gene; (ii) a homozygous null mutation in IL2rg gene; and (iii) a homozygous null mutation in the non-human animal Heme oxygenase-1 (Hmox-1) gene.

### BRIEF DESCRIPTION OF THE DRAWINGS

FIG. 1 shows schematic summaries, not to scale, of genetic engineering of exemplary modified Hmox-1 loci according to certain exemplary embodiments provided herein.

FIG. 2A shows that HMOX-1^{-/-} HIS mice have reduced murine macrophages but no decrease in monocytes in the spleen. Specifically, no significant differences are noted in mouse CD11b+/CD14+ monocytes between HMOX-1^{-/-} and HMOX-1^{+/+} spleen, but HMOX-1^{-/-} have a significant reduction in F4/80+/VCAM+ macrophage subset relative to HMOX-1^{+/+} spleen.

FIG. 2B shows that HMOX-1^{-/-} HIS mice have reduced murine macrophages but no decrease in monocytes in the blood Similar data as FIG. 2A is observed in the blood with reduced F4/80+ macrophage population in HMOX-1−/− blood, despite an increase in CD11b+/CD14+ monocyte population relative to HMOX-1+/+ blood.

FIG. 3 shows that injected human red blood cells (hRBCs) survive longer in HMOX-1^{-/-} mice relative to HMOX-1^{+/+} mice.

FIG. 4A shows that HSC-engrafted HMOX-1^{-/-} HIS mice exhibit hRBCs in peripheral blood.

FIG. 4B shows that both HSC-engrafted HMOX-1^{-/-} and HMOX-1^{+/+} HIS mice exhibit hRBCs in bone marrow (BM), albeit with trending higher levels in HMOX-1^{-/-} HIS mice, indicating that although hRBCs can develop in the BM of HIS mice, HMOX-1^{-/-} HIS mice can allow hRBCs to survive in the peripheral blood.

FIG. 5 shows that injection of human EPO increases hRBC levels in peripheral blood of HMOX-1^{-/-} HIS mice, as measured by percentage of CD235a+ cells.

FIG. 6 shows no indication of kidney/liver damage by serum chemistry in engrafted and non-engrafted HMOX-1^{-/-} HIS mice, as measured by levels of AST, ALP, ALT, and creatine.

FIG. 7 shows that HMOX-1^{-/-} HIS mice have higher engraftment than HSC-donor matched HMOX-1^{+/+} HIS mice. After 12 weeks of engraftment, human CD45+ levels were slightly higher in HMOX-1^{-/-} mice, suggesting that loss of murine macrophages may be potentiating engraftment.

FIG. 8 shows that HMOX-1^{-/-} HIS mice have reduced murine macrophages but no decrease in monocytes in the liver. As the liver is believed the major site of hRBC clearance in mice (Song, Y. et al. Science 371, 1019-1025 (2021)), a reduction of murine F4/80+ macrophages was observed in the liver of HMOX-1^{-/-} relative to HMOX-1^{+/+}, whereas there was a concomitant increase in overall murine CD11b+ monocytes in HMOX-1^{-/-}.

### DETAILED DESCRIPTION

### General

The present disclosure relates to a genetically modified non-human animal (e.g., mouse or rat) comprising: (i) a Rag1 and/or Rag2 gene knock-out; (ii) a IL2rg gene knock-out; and (iii) a homozygous null mutation in the non-human animal Heme oxygenase-1 (Hmox-1) gene. In some embodiments, provided herein is a genetically modified non-human animal (e.g., mouse) comprising: (i) a Rag2 gene knock-out; (ii) a IL2rg gene knock-out; and (iii) a homozygous null mutation in the non-human animal Heme oxygenase-1 (Hmox-1) gene. In some embodiments, provided herein is a genetically modified non-human animal (e.g., rat) comprising: (i) a Rag1 gene knock-out; (ii) a Rag2 gene knock-out; (iii) a IL2rg gene knock-out; and (iv) a homozygous null mutation in the non-human animal Heme oxygenase-1 (Hmox-1) gene. In some embodiments, the genetically modified non-human animal expresses a human or humanized SIRPA protein encoded by a nucleic acid operably linked to a Sirpa promoter. In some embodiments, the genetically modified non-human animal further comprises an inactivation (e.g., a deletion) of fumarylacetoacetase (Fah) gene. In some embodiments, the genetically modified non-human animal further expresses one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to an EPO promoter. In certain embodiments, at least one promoter operably linked to a nucleic acid that encodes a human or humanized protein is an endogenous non-human animal promoter. In other embodiments, the genetically modified animal expresses a human or humanized nucleic acid from the native human promoter and native regulatory elements. The skilled artisan will understand that the genetically modified animal includes genetically modified animals that express at least one human or humanized nucleic acid from any promoter. Examples of promoters useful in the invention include, but are not limited to, DNA pol II promoter, PGK promoter, ubiquitin promoter, albumin promoter, globin promoter, ovalbumin promoter, SV40 early promoter, the Rous sarcoma virus (RSV) promoter, retroviral LTR and lentiviral LTR. Promoter and enhancer expression systems useful in present disclosure also include inducible and/or tissue-specific expression systems.

Rodents, such as mice and rats with components of the human immune system (e.g., HIS mice and rats) hold great promise for studying the human immune system in vivo and for testing human vaccines and testing and developing drugs to treat human diseases and disorders. HIS mice are generated by transplanting a severely immunodeficient mouse strain (such as recombination-activating gene 2 (Rag2) knockout (KO) and interleukin 2 receptor gamma (IL2rg) KO mice) with human hematopoietic stem and progenitor cells (such as CD34+ HSCs). Compared to nonhuman primates, HIS mice have the advantages of a small animal model, i.e., they allow more versatile experimentation, are more accessible to the research community, and are ethically more acceptable than conducting experiments with human subjects. Most importantly, experimental findings derived from HIS mice can be more relevant and applicable to humans Similar rat models that offer such advantages have also been described and are contemplated herein.

Although HIS mice develop human immune cells such as B and T cells, human red blood cells (hRBCs) do not develop in the peripheral blood of HIS mouse models. Human RBC progenitors can develop in the bone marrow of such mice but fail to survive in the periphery. Thus, current HIS mice are not well suited to model diseases or infections related to human red blood cells (hRBCs) or to test effects of therapeutic drugs on human red blood cells.

To overcome these limitations, the present disclosure provides Hmox-1-deficient HIS mouse models. Infused human RBCs are rapidly cleared from mice and their destruction is primarily believed to be due to phagocytosis by murine macrophages. This idea is supported by the fact that elimination of macrophages with in vivo clodronate liposome treatment extends survival of human RBCs. However, this approach requires repeated injections, which are not feasible due to murine toxicity. Hmox-1 deficiency in mice results in loss of erythrophagocytic macrophages, i.e., macrophages that eat red blood cells. This is because the Hmox-1 enzyme is necessary for macrophages to breakdown and recycle heme from engulfed RBCs. With the loss of this enzyme, toxic heme builds up in erythrophagocytic macrophages resulting in their death. Thus, Hmox-1 KO in HIS model allows for the survival of human RBCs upon HSC engraftment. It was demonstrated herein that Hmox-1 deficient hSIRPA Rag2^{-/-} IL-2Rγ^{-/-} mice exhibited human RBCs in the peripheral blood, in contrast to HSC donor-matched Hmox-1 competent control mice. This new model provides a useful tool to study human RBC biology as well as a useful model for developing treatments for RBC diseases.

The genetically modified non-human animals provided herein find many uses in the art, including, for example, in modeling human erythropoiesis and erythrocyte function; in modeling human pathogen infection of erythrocytes; in in vivo screens for agents that modulate erythropoiesis and/or erythrocyte function, e.g., in a healthy or a diseased state; in in vivo screens for agents that are toxic to erythrocytes or erythrocyte progenitors; in in vivo screens for agents that prevent against, mitigate, or reverse the toxic effects of toxic agents on erythrocytes or erythrocyte progenitors; in in vivo screens of erythrocytes or erythrocyte progenitors from an individual to predict the responsiveness of an individual to a disease therapy. These and other objects, advantages, and features of the invention will become apparent to those persons skilled in the art upon reading the details of the compositions and methods as more fully described below.

### Definitions

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

A “coding region” of a gene includes the nucleotide residues of the coding strand of the gene and the nucleotides of the non-coding strand of the gene which are homologous with or complementary to, respectively, the coding region of an mRNA molecule which is produced by transcription of the gene. A “coding region” of a mRNA molecule also includes the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

As used herein, the phrase “endogenous gene” or “endogenous gene segment” refers to a gene or gene segment found in a parent or reference organism prior to introduction of a disruption, deletion, replacement, alteration, or modification as described herein. In some embodiments, a reference organism is a wild-type organism. In some embodiments, a reference organism is an engineered organism. In some embodiments, a reference organism is a laboratory-bred organism (whether wild-type or engineered).

As used herein, the term “chimeric” refers to nucleic acids or proteins whose structures (i.e., nucleotide or amino acid sequences) include portions that are from different species. In some embodiments, the “chimeric” nucleic acids or proteins described herein include nucleotide or amino acid sequences that are from both a non-human source and a human. In such embodiments, the “chimeric” nucleic acids or proteins can also be referred to as “humanized” nucleic acids or protein.

As used herein, the term “correspond to” refers to exons that encode the same or homologous functional domain or portion of a protein. For example, “exons that correspond to mouse Hmox-1 exons 3-5” refers to the exons from the genetically modified non-human animal that encode the same or homologous functional domain or portion of the protein as encoded by the mouse exons 3-5. These could be exons 3-5 of the genetically modified non-human animal, or other exons due to the difference in exon configuration among different non-human animal species.

The term “humanized”, is used herein in accordance with its art-understood meaning to refer to nucleic acids or proteins whose structures (i.e., nucleotide or amino acid sequences) include portions that are from a non-human source, which are engineered to have a structure and function more similar to true human nucleic acids or proteins than the original source nucleic acids or proteins. For example, humanizing can involve selecting amino acid substitutions to make a non-human sequence more similar to a human sequence. Humanizing can also involve grafting at least a portion of a non-human protein into a human protein. To give but one example, in the case of a membrane receptor, a “humanized” gene may encode a polypeptide having an extracellular portion having an amino acid sequence as that of a human extracellular portion and the remaining sequence as that of a non-human (e.g., mouse) polypeptide. In some embodiments, a humanized gene comprises at least a portion of a DNA sequence of a human gene. In some embodiments, a humanized protein comprises a sequence having a portion that appears in a human protein. The term “human” is art recognized, and refers to nucleic acids or proteins whose structures (i.e., nucleotide or amino acid sequences) are entirely from a human source.

As used herein, the term “locus” refers to a location on a chromosome that contains a set of related genetic elements (e.g., genes, gene segments, regulatory elements). A locus can be endogenous or non-endogenous. The term “endogenous locus” refers to a location on a chromosome at which a particular genetic element is naturally found. In some embodiments, an endogenous locus has a sequence found in nature. In some embodiments, an endogenous locus is a wild-type locus. In some embodiments, an endogenous locus is an engineered locus.

The phrase “non-human animal” as used herein refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human mammal is a primate, a goat, a sheep, a pig, a dog, a cow, or a rodent. In some embodiments, a non-human animal is a rodent such as a rat or a mouse.

The phrase “operably linked”, as used herein, refers to a juxtaposition wherein the components described are in a relationship permitting them to function in their intended manner A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to affect the expression and processing of coding sequences to which they are ligated. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “polypeptide”, as used herein, refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man.

The term “promoter” as used herein includes a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors. The phrase “endogenous promoter” refers to a promoter that is naturally associated, e.g., in a wild-type organism, with an endogenous gene.

The term “recombinant”, as used herein, is intended to refer to polypeptides (e.g., signal-regulatory proteins as described herein) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597, Little M. et al (2000) Immunology Today 21:364-370) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant polypeptide is comprised of sequences found in the genome of a source organism of interest (e.g., human, mouse, etc.). In some embodiments, a recombinant polypeptide has an amino acid sequence that resulted from mutagenesis (e.g., in vitro or in vivo, for example in a non-human animal), so that the amino acid sequences of the recombinant polypeptides are sequences that, while originating from and related to polypeptides sequences, may not naturally exist within the genome of a non-human animal in vivo.

The term “replacement” is used herein to refer to a process through which a “replaced” nucleic acid sequence (e.g., a gene) found in a host locus (e.g., in a genome) is removed from that locus and a different, “replacement” nucleic acid is located in its place. In some embodiments, the replaced nucleic acid sequence and the replacement nucleic acid sequences are comparable to one another in that, for example, they are homologous to one another and/or contain corresponding elements (e.g., protein-coding elements, regulatory elements, etc.). In some embodiments, a replaced nucleic acid sequence includes one or more of a promoter, an enhancer, a splice donor site, a splice receiver site, an intron, an exon, an untranslated region (UTR); in some embodiments, a replacement nucleic acid sequence includes one or more coding sequences. In some embodiments, a replacement nucleic acid sequence is a homolog of the replaced nucleic acid sequence. In some embodiments, a replacement nucleic acid sequence is an ortholog of the replaced sequence. In some embodiments, a replacement nucleic acid sequence is or comprises a human nucleic acid sequence. In some embodiments, including where the replacement nucleic acid sequence is or comprises a human nucleic acid sequence, the replaced nucleic acid sequence is or comprises a rodent sequence (e.g., a mouse sequence). The nucleic acid sequence so placed may include one or more regulatory sequences that are part of source nucleic acid sequence used to obtain the sequence so placed (e.g., promoters, enhancers, 5′- or 3′-untranslated regions, etc.). For example, in various embodiments, the replacement is a substitution of an endogenous sequence with a heterologous sequence that results in the production of a gene product from the nucleic acid sequence so placed (comprising the heterologous sequence), but not expression of the endogenous sequence; the replacement is of an endogenous genomic sequence with a nucleic acid sequence that encodes a protein that has a similar function as a protein encoded by the endogenous sequence. In various embodiments, an endogenous gene or fragment thereof is replaced with a corresponding human gene or fragment thereof. A corresponding human gene or fragment thereof is a human gene or fragment that is an ortholog of, or is substantially similar or the same in structure and/or function, as the endogenous gene or fragment thereof that is replaced.

“Variant” as the term is used herein, includes a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

The term “vector”, as used herein, refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

The term “wild-type”, as used herein, has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

### Genetically Modified Loci

In certain aspects provided herein are genetically modified non-human animals (e.g., mice or rats) comprising: (i) a Rag1 and/or Rag2 gene knock-out; (ii) a IL2rg gene knock-out; and (iii) a homozygous null mutation in the non-human animal Heme oxygenase-1 (Hmox-1) gene. In some embodiments, provided herein are genetically modified animals (e.g., mice) comprising: (i) a Rag2 gene knock-out; (ii) a IL2rg gene knock-out; and (iii) a homozygous null mutation in the mouse Hmox-1 gene. In some embodiments, provided herein are genetically modified animals (e.g., rats) comprising: (i) a Rag1 gene knock-out; (ii) a Rag 2 gene knock-out; (iii) a IL2rg gene knock-out; and (iv) a homozygous null mutation in the rat Hmox-1 gene. In some embodiments, the genetically modified non-human animal expresses a human or humanized SIRPA protein encoded by a nucleic acid operably linked to a Sirpa promoter. In certain embodiments, the genetically modified non-human animal further expresses one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to an EPO promoter. In some embodiments, the genetically modified non-human animal further comprises an inactivation (e.g., deletion) of fumarylacetoacetase (FAH) gene. In certain embodiments, at least one promoter operably linked to a nucleic acid that encodes a human or humanized protein is an endogenous non-human animal promoter. In some embodiments, the genetically modified non-human animal comprises engraftment of human hematopoietic stem cells (HSC). In some embodiments, the genetically modified non-human animal comprises engraftment of human liver cells.

### Hmox-1 Knockout

In certain aspects, genetically modified non-human animals (e.g., mice or rats) provided herein comprise a homozygous null mutation in the non-human animal Heme oxygenase-1 (Hmox-1) gene.

Heme oxygenase metabolizes heme and releases free iron, carbon monoxide, and biliverdin, which quickly undergoes conversion to bilirubin. Humans and mice contain two well-characterized heme oxygenase enzymes: HMOX-1, which is inducible, and HMOX-2, which is constitutively expressed in most tissues (Kovtunovych et al. (2010) Blood 116:6054-6062). Previous work in mouse models has shown that the lack of both Hmox-1 and Hmox-2 is embryonically lethal. A HMOX-1 deficient human patient died at 6 years of age and presented with growth retardation, severe anemia, endothelial cell damage, fibrosis of spleen, liver, and kidney, and iron overload in liver and kidney (Kawashima et al. (2002) Hum. Path. 33:125-130; Yachie et al. (1999) J. Clin. Invest. 103:129-135). Hmox-1^{-/-} mice are anemic, but oxidatively-stressed RBCs have longer lifespan. Splenomegaly from increased red pulp was observed in Hmox-1^{-/-} mice, and older mice have splenic fibrosis. Iron build-up in kidneys in Hmox-1^{-/-} mice caused kidney damage in these mice. Macrophage-deficiency in spleen and liver was also observed in Hmox-1^{-/-} mice (Poss et al. (1997) PNAS 94:10919-10924; Kovtunovych et al. (2010) Blood 116:6054-6062; Kovtunovych et al. (2014) Blood 124:1522-1530). For example, Hmox-1^{-/-} mice have decreased macrophages (F4/80⁺) in spleen, liver, blood and bone marrow (BM), but still have myeloid cells (CD11b⁺) (Fraser et al. (2015) Haematologica 100:601-610). Lack of Hmox-1 renders erthyrophagocytic macrophages unable to process heme and causes intracellular toxicity due to heme build-up (Kovtunovych et al. (2010) Blood 116:6054-6062). Wild-type bone marrow transplantation reverses disease in Hmox-1^{-/-} mice as a result of restoration of heme recycling by repopulation of the tissues with wild-type macrophages (Kovtunovych et al. (2014) Blood 124:1522-1530).

Representative human HMOX-1 cDNA and human HMOX-1 protein sequences are well-known in the art and are publicly available from the National Center for Biotechnology Information (NCBI). For example, human (NP_002124.1) is encodable by the transcript (NM_002133.3). Nucleic acid and polypeptide sequences of Hmox-1 orthologs in organisms other than humans are well-known and include, for example, chimpanzee Hmox-1 (XM_525579.6 and XP_525579.2), Rhesus monkey Hmox-1 (XM_028827760.1 and XP_028683593.1), cattle Hmox-1 (NM_001014912.1 and NP_001014912.1), dog Hmox-1 (NM_001194969.1 and NP_001181898.1), rat Hmox-1 (NM_012580.2 and NP_036712.1), mouse Hmox-1 (NM_010442.2 and NP_034572.1), Chinese hamster Hmox-1 (XM_003511957.5 and XP_003512005.1), chicken Hmox-1 (XM_046921508.1 and XP_046777464.1), tropical clawed frog Hmox-1 (XM_002934720.5 and XP_002934766.2), and zebrafish Hmox-1 (NM_001127516.1 and NP_001120988.1).

In certain aspects, the genetically modified non-human animal comprises a homozygous null mutation in the non-human animal Heme oxygenase-1 (Hmox-1) gene. A null mutation comprises a deletion, an insertion, and/or a substitution in a gene which leads to no functional gene product (e.g., complete absence of the gene product (protein, RNA) at the molecular level, or the expression of a non-functional gene product). In the present disclosure, a null mutation has the same meaning and is used interchangeably with an inactivating mutation. A homozygous null mutation refers to having a null mutation in all alleles. For example, a homozygous null mutation in mouse or rat Hmox-1 gene refers to having a null mutation in two alleles (i.e., two null alleles) for mouse or rat Hmox-1 gene. A gene with a homozygous null mutation is also referred to as a gene knockout or a gene deficient/deficiency. For example, a homozygous null mutation in Hmox-1 is also referred to as a Hmox-1 knockout or a Hmox-1-deficiency. Thus, the null mutation in the non-human animal Hmox-1 gene comprises a deletion, an insertion, and/or a substitution in the non-human animal Hmox-1 gene. In some instances, the endogenous non-human animal Hmox-1 locus comprises a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some embodiments, the null mutation is a deletion of at least exons that correspond to mouse Hmox-1 exons 3-5. In some embodiments, the null mutation is a deletion of the full Hmox-1 endogenous coding sequence. In some embodiments, the non-human animals provided herein do not express Hmox-1 protein.

In some embodiments, the homozygous null mutation in the non-human animal Hmox-1 gene comprises the same null mutation for all the alleles. In some embodiments, the homozygous null mutation in the non-human animal Hmox-1 gene comprises different null mutations for different alleles.

Mouse Hmox-1 is located on Chromosome 8, GRCm39, NC_000074.7 (75820246-75827221), and the mouse Hmox-1 coding sequence may be found at Genbank Accession No. NM_010442.2. The mouse Hmox-1 locus includes 5 exons, with exons 1-5 being coding exons. As such, in some embodiments, the genetically modified animals provided herein are mice, and one or more of exons 1-5 of the mouse Hmox-1 gene are deleted or mutated in the genetically modified mice. In some instances, other aspects of the genomic locus of the mouse Hmox-1 gene, e.g., introns, 3′ and/or 5′ untranslated sequence (UTRs) are also deleted or mutated. In some instances, the whole regions of the mouse Hmox-1 genomic locus are deleted. In some embodiments, the whole genomic region from the start codon to the stop codon of the mouse Hmox-1 gene is deleted. For example, the genetically modified mice may comprise a deletion of ˜7 kb of mouse sequence (GRCm38 coordinates chr8: 75093750-75100019) as illustrated in Example 1.

The deleted, modified or altered Hmox-1 gene at the endogenous Hmox-1 locus can be detected using a variety of methods including, for example, PCR, Western blot, Southern blot, restriction fragment length polymorphism (RFLP), or a gain or loss of allele assay. In some embodiments, the non-human animal is homozygous for the deletion or null mutation of the endogenous Hmox-1 gene.

In some embodiments, the non-human animal (e.g., mouse or rat) comprising a homozygous null mutation in Hmox-1 gene, i.e., the Hmox-1 deficient non-human animal, is an immunocompromised animal. For example, the Hmox-1 deficient non-human animal (e.g., mouse or rat) may include at least one null allele for the Rag2 gene (“recombination activating gene 2”, wherein the coding sequence for the mouse gene may be found at Genbank Accession No. NM_009020.3). In some embodiments, the Hmox-1 deficient non-human animal (e.g., mouse or rat) includes two null alleles for Rag2. In other words, the Hmox-1 deficient non-human animal (e.g., mouse or rat) is homozygous null for Rag2. In other embodiments, Hmox-1 deficient non-human animal (e.g., mouse or rat) includes one or two null alleles for Rag1 gene. In some embodiments, the Hmox-1 deficient non-human animal (e.g., mouse or rat) is homozygous null for Rag1. In some embodiments, Hmox-1 deficient non-human animal (e.g., mouse or rat) includes (i) one or two null alleles for Rag1 gene; and (ii) one or two null alleles for Rag2 gene. In some embodiments, the Hmox-1 deficient non-human animal (e.g., mouse or rat) is homozygous null for both Rag1 and Rag2. In some embodiments, the Hmox-1 deficient non-human animal is an immunocompromised mouse comprising two null alleles (i.e., homozygous null) for Rag2. In some embodiments, the Hmox-1 deficient non-human animal is an immunocompromised rat comprising two null alleles (i.e., homozygous null) for Rag1 and two null alleles (i.e., homozygous null) for Rag2. As another example, the Hmox-1 deficient non-human animal (e.g., mouse or rat) includes at least one null allele for the IL2rg gene (“interleukin 2 receptor, gamma”, also known as the common gamma chain, or γC, wherein the coding sequence for the mouse gene may be found at Genbank Accession No. NM 013563.4). In some embodiments, the Hmox-1 deficient non-human animal (e.g., mouse or rat) includes two null alleles for IL2rg. In other words, the Hmox-1 deficient non-human animal (e.g., mouse or rat) is homozygous null for IL2rg, i.e., it is IL2rg^{-/-} (or IL2rg^{Y/-} where the IL2rg gene is located on the X chromosome as in mouse). In some embodiments, the Hmox-1 deficient non-human animal (e.g., mouse or rat) includes a null allele for both Rag2 and IL2rg, i.e., it is Rag2^{-/-} IL2rg^{-/-} (or Rag2^{-/-} IL2rg^{Y/-} where the IL2rg gene is located on the X chromosome as in mouse or rat). In some embodiments, the Hmox-1 deficient non-human animal (e.g., mouse or rat) includes a null allele for both Rag1 and IL2rg. In some embodiments, the Hmox-1 deficient non-human animal (e.g., mouse or rat) includes a null allele for Rag1, Rag2, and IL2rg. In some embodiments, the Hmox-1 deficient non-human animal is an immunocompromised mouse comprising two null alleles (i.e., homozygous null) for Rag2 and two null alleles (i.e., homozygous null) for IL2rg (or one null allele for male mice). In some embodiments, the Hmox-1 deficient non-human animal is an immunocompromised rat comprising two null alleles (i.e., homozygous null) for Rag1, two null alleles (i.e., homozygous null) for Rag2, and two null alleles (i.e., homozygous null) for IL2rg (or one null allele for male rat). Other genetic modifications are also contemplated. For example, the Hmox-1 deficient non-human animal (e.g., mouse or rat) may include modifications in other genes associated with the development and/or function of hematopoietic cells and the immune system, e.g., the replacement of one or more other non-human animal genes with nucleic acid sequence(s) encoding human or humanized polypeptides. Such genes include but are not limited to, e.g., SIRPA, CD47, M-CSF, GM-CSF, TPO, EPO, IL-3, and IL-15. Additionally or alternatively, the Hmox-1 deficient non-human animal (e.g., mouse or rat) may include modifications in genes associated with the development and/or function of other cells and tissues, e.g., genes associated with human disorders or disease, or genes that, when modified in a non-human animal, e.g., mice, provide for models of human disorders and disease. In one embodiment described herein below, the Hmox-1 deficient non-human animal (e.g., mouse or rat) may include a mutation, insertion, or deletion in Fah gene. Introduction of other genetic modifications may be accomplished by either ES cell modification and/or breeding. For example, the Hmox-1 deficient (and, optionally, Rag2 and IL2rg deficient) non-human animal (e.g., mouse or rat) may be bred with a non-human animal that comprises one or more other genetic modifications, including but not limited to, e.g., a modification in SIRPA, CD47, M-CSF, GM-CSF, TPO, EPO, IL-3, IL-15, and/or Fah gene. In some embodiments, all genetic modifications are bred to homozygous in the genetically modified animal described herein.

### Immunodeficient Non-Human Animals

As explained above, genetically modified non-human animals comprising Hmox-1 deficiency described herein are also immunodeficient because they comprise a deficiency in Rag1 and/or Rag2, and Il2rg genes. Rag1, Rag2, and Il2rg are essential components of the adaptive immune system. When one or more of these genes are mutated in animals, T-cells and B-cells do not mature, and the animals are severely compromised. When these animals are challenged with xenotransplanted cells, they are unable to mount an immune response to the foreign cells.

V(D)J recombination-activating protein 1 (also known as RAG1, recombination-activating 1, recombination activating gene 1, and recombination activating protein 1) is encoded by the Rag1 gene (also known as recombination activating 1). RAG1 is a catalytic component of the RAG complex, a multiprotein complex that mediates the DNA cleavage phase during V(D)J recombination. V(D)J recombination assembles a diverse repertoire of immunoglobulin and T-cell receptor genes in developing B and T-lymphocytes through rearrangement of different V (variable), in some cases D (diversity), and J (joining) gene segments. In the RAG complex, RAG1 mediates the DNA-binding to the conserved recombination signal sequences (RSS) and catalyzes the DNA cleavage activities by introducing a double-strand break between the RSS and the adjacent coding segment. RAG2 is not a catalytic component but is required for all known catalytic activities. RAG1 and RAG2 are essential to the generation of mature B cells and T cells, two types of lymphocytes that are crucial components of the adaptive immune system.

Mouse Rag1 maps to 2 E2; 2 53.88 cM on chromosome 2 (NCBI RefSeq Gene ID 19373; Assembly GRCm39 (GCF_000001635.27); location NC_000068.8 (101468597 . . . 101479877, complement). Reference to the mouse Rag1 gene includes the canonical, wild type form as well as all allelic forms and isoforms. The canonical, wild type mouse RAG1 protein has been assigned UniProt accession number P15919 and NCBI Accession No. NP_033045.2. Reference to mouse RAG1 proteins includes wild type forms as well as all allelic forms and isoforms. An mRNA (cDNA) encoding the canonical isoform is assigned NCBI Accession No. NM_009019.2. Reference to the mouse Rag1 mRNA (cDNA) and coding sequence includes the canonical, wild type forms as well as all allelic forms and isoforms.

Rat Rag1 maps to 3q31 on chromosome 3 (NCBI RefSeq Gene ID 84600; Assembly mRatBN7.2 (GCF_015227675.2); location NC_051338.1 (87917061 . . . 87928158, complement). Reference to the rat Rag1 gene includes the canonical, wild type form as well as all allelic forms and isoforms. The canonical, wild type rat RAG1 protein has been assigned UniProt accession number G3V6K9 and NCBI Accession No. NP_445920.1. Reference to rat RAG1 proteins includes canonical, wild type forms as well as all allelic forms and isoforms. An mRNA (cDNA) encoding the canonical isoform is assigned NCBI Accession No. NM_053468.1. Reference to the rat Rag1 mRNA (cDNA) and coding sequence includes the canonical, wild type forms as well as all allelic forms and isoforms.

An inactivated endogenous Rag1 gene is a Rag1 gene that does not produce a RAG1 protein or does not produce a functional RAG1 protein. The non-human animal (or cell or genome) can comprise the inactivated Rag1 gene in its germline. The non-human animal (or cell or genome) can be homozygous for an inactivating mutation in the Rag1 gene. As one example, an inactivated endogenous Rag1 gene can comprise an insertion, a deletion, or one or more point mutations in the endogenous Rag1 gene resulting in loss of expression of functional RAG1 protein. Some inactivated endogenous Rag1 genes can comprise a deletion or disruption of all of the endogenous Rag1 gene or can comprise a deletion or disruption of a fragment of (i.e., a part of or portion of) the endogenous Rag1 gene. For example, some, most, or all of the coding sequence in the endogenous Rag1 gene can be deleted or disrupted. In one example, a 5′ fragment of the Rag1 gene can be deleted or disrupted (e.g., including the start codon). As one example, an inactivated endogenous Rag1 gene can be one in which the start codon of the endogenous Rag1 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag1 gene can be deleted or disrupted (e.g., including the stop codon). In another example, an internal fragment of the Rag1 gene (i.e., a fragment from the middle of the Rag1 gene) can be deleted or disrupted. In another example, all of the coding sequence in the endogenous Rag1 gene is deleted or disrupted.

V(D)J recombination-activating protein 2 (also known as RAG2, recombination-activating 2, recombination activating gene 2, and recombination activating protein 2) is encoded by the Rag2 gene (also known as recombination activating 2). As mentioned above, RAG1 is a catalytic component of the RAG complex, a multiprotein complex that mediates the DNA cleavage phase during V(D)J recombination. RAG2 is not a catalytic component but is required for all known catalytic activities. RAG1 and RAG2 are essential to the generation of mature B cells and T cells, two types of lymphocyte that are crucial components of the adaptive immune system.

Mouse Rag2 maps to 2 E2; 2 53.87 cM on chromosome 2 (NCBI RefSeq Gene ID 19374; Assembly GRCm39 (GCF_000001635.27); location NC_000068.8 (101455057 . . . 101462873). Reference to the mouse Rag2 gene includes the canonical, wild type form as well as all allelic forms and isoforms. The canonical, wild type mouse RAG2 protein has been assigned UniProt accession number P21784 and NCBI Accession No. NP_033046.1. Reference to mouse RAG2 proteins includes canonical, wild type forms as well as all allelic forms and isoforms. An mRNA (cDNA) encoding the canonical isoform is assigned NCBI Accession No. NM_009020.3. Reference to the mouse Rag2 mRNA (cDNA) and coding sequence includes the canonical, wild type forms as well as all allelic forms and isoforms.

Rat Rag2 maps to 3q31 on chromosome 3 (NCBI RefSeq Gene ID 295953; Assembly mRatBN7.2 (GCF_015227675.2); location NC_051338.1 (87902373 . . . 87910227). Reference to the rat Rag2 gene includes the canonical, wild type form as well as all allelic forms and isoforms. The canonical, wild type rat RAG2 protein has been assigned UniProt accession number G3V6K7 and NCBI Accession No. NP_001093998.1. Reference to rat RAG2 proteins includes canonical, wild type forms as well as all allelic forms and isoforms. An mRNA (cDNA) encoding the canonical isoform is assigned NCBI Accession No. NM_001100528.1. Reference to the rat Rag2 mRNA (cDNA) and coding sequence includes the canonical, wild type forms as well as all allelic forms and isoforms.

An inactivated endogenous Rag2 gene is a Rag2 gene that does not produce a RAG2 protein or does not produce a functional RAG2 protein. The non-human animal (or cell or genome) can comprise the inactivated Rag2 gene in its germline. The non-human animal (or cell or genome) can be homozygous for an inactivating mutation in the Rag2 gene. As one example, an inactivated endogenous Rag2 gene can comprise an insertion, a deletion, or one or more point mutations in the endogenous Rag2 gene resulting in loss of expression of functional RAG2 protein. Some inactivated endogenous Rag2 genes can comprise a deletion or disruption of all of the endogenous Rag2 gene or can comprise a deletion or disruption of a fragment of (i.e., a part of or portion of) the endogenous Rag2 gene. For example, some, most, or all of the coding sequence in the endogenous Rag2 gene can be deleted or disrupted. In one example, a 5′ fragment of the Rag2 gene can be deleted or disrupted (e.g., including the start codon). As one example, an inactivated endogenous Rag2 gene can be one in which the start codon of the endogenous Rag2 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag2 gene can be deleted or disrupted (e.g., including the stop codon). In another example, an internal fragment of the Rag2 gene (i.e., a fragment from the middle of the Rag2 gene) can be deleted or disrupted. In another example, all of the coding sequence in the endogenous Rag2 gene is deleted or disrupted.

Interleukin 2 receptor subunit gamma (also known as interleukin 2 receptor, gamma; interleukin 2 receptor, gamma (severe combined immunodeficiency), isoform CRA_a; cytokine receptor common subunit gamma precursor) is encoded by the Il2rg gene (also known as interleukin 2 receptor subunit gamma or IL2RG). IL2RG is a cytokine receptor subunit that is common to receptor complexes for several different interleukin receptors. IL2RG is located on the surface of immature blood-forming cells in bone marrow. IL2RG partners with other proteins to direct blood-forming cells to form lymphocytes. IL2RG also directs the growth and maturation of T cells, B cells, and natural killer cells. Mutations in Il2rg can cause X-linked severe combined immunodeficiency in which lymphocytes cannot develop normally. A lack of functional mature lymphocytes disrupts the immune system's ability to protect the body from infection.

Mouse Il2rg maps to X D; X 43.9 cM on chromosome X (NCBI RefSeq Gene ID 16186; Assembly GRCm39 (GCF_000001635.27); location NC_000086.8 (100307991 . . . 100311861, complement). Reference to the mouse Il2rg gene includes the canonical, wild type form as well as all allelic forms and isoforms. The canonical, wild type mouse IL2rG protein has been assigned UniProt accession number P34902 and NCBI Accession No. NP_038591.1. Reference to mouse IL2RG proteins includes canonical, wild type forms as well as all allelic forms and isoforms. An mRNA (cDNA) encoding the canonical isoform is assigned NCBI Accession No. NM_013563.4. Reference to the mouse Il2rg mRNA (cDNA) and coding sequence includes the canonical, wild type forms as well as all allelic forms and isoforms.

Rat Il2rg maps to Xq22 on chromosome X (NCBI RefSeq Gene ID 140924; Assembly mRatBN7.2 (GCF_015227675.2); location NC_051356.1 (66395330 . . . 66399026, complement). Reference to the rat Il2rg gene includes the canonical, wild type form as well as all allelic forms and isoforms. The canonical, wild type rat IL2rG protein has been assigned UniProt accession number Q68FU6 and NCBI Accession No. NP_543165.1. Reference to rat IL2RG proteins includes canonical, wild type forms as well as all allelic forms and isoforms. An mRNA (cDNA) encoding the canonical isoform is assigned NCBI Accession No. NM_080889.1. Reference to the rat Il2rg mRNA (cDNA) and coding sequence includes the canonical, wild type forms as well as all allelic forms and isoforms.

An inactivated endogenous Il2rg gene is an Il2rg gene that does not produce a IL2RG protein or does not produce a functional IL2RG protein. The non-human animal (or cell or genome) can comprise the inactivated Il2rg gene in its germline. The non-human animal (or cell or genome) can be homozygous for an inactivating mutation in the Il2rg gene. As one example, an inactivated endogenous Il2rg gene can comprise an insertion, a deletion, or one or more point mutations in the endogenous Il2rg gene resulting in loss of expression of functional IL2RG protein. Some inactivated endogenous Il2rg genes can comprise a deletion or disruption of all of the endogenous Il2rg gene or can comprise a deletion or disruption of a fragment of (i.e., a part of or portion of) the endogenous Il2rg gene. For example, some, most, or all of the coding sequence in the endogenous Il2rg gene can be deleted or disrupted. In one example, a 5′ fragment of the Il2rg gene can be deleted or disrupted (e.g., including the start codon). As one example, an inactivated endogenous Il2rg gene can be one in which the start codon of the endogenous Il2rg gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Il2rg gene can be deleted or disrupted (e.g., including the stop codon). In another example, an internal fragment of the Il2rg gene (i.e., a fragment from the middle of the Il2rg gene) can be deleted or disrupted. In another example, all of the coding sequence in the endogenous Il2rg gene is deleted or disrupted.

### Humanized Sirpa Loci

In certain aspects, the genetically modified non-human animals provided herein further express a human or humanized SIRPA protein encoded by a nucleic acid operably linked to a Sirpa promoter.

Signal regulatory proteins (SIRPs) constitute a family of cell surface glycoproteins which are expressed on lymphocytes, myeloid cells (including macrophages, neutrophils, granulocytes, myeloid dendritic cells, and mast cells) and neurons (e.g., see Barclay and Brown, 2006, Nat Rev Immunol 6, 457-464). The reported SIRP genes include at least SIRPA, SIRP3, SIRPβ, SIRPγ, and SIRP8 and can be categorized by their respective ligands and types of signaling in which they are involved. SIRPA (also referred to as CD172A, SHPS1, P84, MYD-1, BIT and PTPNS1) is expressed on immune cells of the myeloid lineage and functions as an inhibitory receptor via an immunoreceptor tyrosine-based inhibitory motif (ITIM). SIRPA expression has also been observed on neurons. Reported ligands for SIRPA include, most notably, CD47, but also include surfactant proteins A and D. The role of SIRPA, in particular, has been investigated in respect of its inhibitory role in the phagocytosis of host cells by macrophages. For example, CD47 binding to SIRPA on macrophages triggers inhibitory signals that negatively regulates phagocytosis. Alternatively, positive signaling effects mediated through SIRPA binding have been reported (Shultz et al., 1995, J Immunol 154, 180-91). SIRPA has been shown to improve cell engraftment in immunodeficient mice (Strowig et al. Proc Natl Acad Sci USA 2011; 108: 13218-13223).

Polypeptide sequences for wild-type human SIRPA and the nucleic acid sequences that encode wild-type human SIRPA may be found at Genbank Accession Nos. NP_001035111.1 and NM_001040022.1 (isoform 1 and transcript variant 1); NP_001035112.1 and NM_001040023.2 (isoform 1 and transcript variant 2); NP_001317657.1 and NM_001330728.1 (isoform 2 and transcript variant 4); and NP_542970.1 and NM_080792.3 (isoform 1 and transcript variant 3). The SIRPA gene is conserved in at least chimpanzee, Rhesus monkey, dog, cow, mouse, rat, and chicken. The genomic locus encoding the wild-type human SIRPA protein may be found in the human genome at Chromosome 20; NC_000020.11 (1894167-1940592). In some embodiments, human SIRPA protein is encoded by exons 2 through 9 at this locus. As such, in some embodiments, a nucleic acid sequence including coding sequence for human SIRPA includes one or more of exons 2-9 of the human SIRPA gene. In some instances, the nucleic acid sequence also includes aspects of the genomic locus of the human SIRPA, e.g., introns, 3′ and/or 5′ untranslated sequence (UTRs). In some instances, the nucleic acid sequence includes whole regions of the human SIRPA genomic locus. In some instances, the nucleic acid sequence includes exons 2-4 of the human SIRPA genomic locus.

Exemplary humanized Sirpa sequences are set forth in Table 3. For protein sequences, signal peptides are underlined, and transmembrane and cytoplasmic sequences are italicized. Representative mouse Sirpa cDNA, mouse Sirpa protein, human SIRPA cDNA, and human SIRPA protein sequences are described in U.S. Pat. No. 11,019,810, which is incorporated by reference herein in its entirety.

TABLE 3
Humanized Sirpa Protein (SEQ ID NO: 1)

MEPAGPAPGRLGPLLLCLLLSASCFCTGVAGEEELQVIQPDKSVLVAAGETATLRCTATSLIPVGPIQWER
GAGPGRELIYNQKEGHFPRVTTVSDLTKRNNMDFSIRIGNITPADAGTYYCVKFRKGSPDDVEFKSGAGT
ELSVRAKPSAPVVSGPAARATPQHTVSFTCESHGFSPRDITLKWFKNGNELSDFQTNVDPVGESVSYSIH
STAKVVLTREDVHSQVICEVAHVTLQGDPLRGTANLSETIRVPPTLEVTQQPVRAENQVNVTCQVRKFYP
QRLQLTWLENGNVSRTETASTVTENKDGTYNWMSWLLVNVSAHRDDVKLTCQVEHDGQPAVSKSHDLKVS
AHPKEQGSNTAADNNATHNWNVFIGVGVACALLVVLLMAALYLLRIKQKKAKGSTSSTRLHEPEKNAR
EITQIQDINDINDITYADLNLPKEKKPAPRAPEPNNHTEYASIETGKVPRPEDTLTYADLDMVHLSRAQP
APKPEPSFSEYASVQVQRK

In some embodiments, the non-human animals provided herein express humanized Sirpa proteins on the surface of immune cells (e.g., myeloid cells) of the non-human animals resulting from a genetic modification of an endogenous locus of the non-human animal that encodes a Sirpa protein. Suitable examples described herein include rodents, for example, mice.

A humanized Sirpa gene, in some embodiments, comprises genetic material from a heterologous species (e.g., humans), wherein the humanized Sirpa gene encodes a Sirpa protein that comprises the encoded portion of the genetic material from the heterologous species. In some embodiments, a humanized Sirpa gene of the present disclosure comprises genomic DNA of a heterologous species that corresponds to the extracellular portion of a SIRPA protein that is expressed on the plasma membrane of a cell. Non-human animals, embryos, cells and targeting constructs for making non-human animals, non-human embryos, and cells containing said humanized Sirpa gene are also provided.

In some embodiments, an endogenous non-human animal (e.g., rodent) Sirpa gene is deleted. In some embodiments, an endogenous non-human animal (e.g., rodent) Sirpa gene is altered, wherein a portion of the endogenous non-human animal (e.g., rodent) Sirpa gene is replaced with a heterologous sequence (e.g., a human SIRPA sequence in whole or in part). In some embodiments, all or substantially all of an endogenous non-human animal (e.g., rodent) Sirpa gene is replaced with a heterologous gene (e.g., a human SIRPA gene). In some embodiments, a portion of a heterologous SIRPα gene is inserted into an endogenous non-human Sirpa gene at an endogenous non-human animal (e.g., rodent) Sirpa locus. In some embodiments, the heterologous gene is a human gene. In some embodiments, the modification or humanization is made to one of the two copies of the endogenous non-human animal (e.g., rodent) Sirpa gene, giving rise to a non-human animal which is heterozygous with respect to the humanized Sirpa gene. In other embodiments, a non-human animal is provided that is homozygous for a humanized Sirpa gene. In some embodiments, all of an endogenous non-human animal (e.g., rodent) Sirpa gene is replaced with a portion of heterologous gene (e.g., a portion of human SIRPA gene), such that the genetically modified non-human animal (e.g., rodent) expresses a functional fragment of a full-length human SIRPA polypeptide (e.g., an extracellular domain of a human SIRPA polypeptide).

A non-human animal of the present disclosure contains a human SIRPA gene in whole or in part at an endogenous non-human Sirpa locus. Thus, such non-human animals can be described as having a heterologous SIRP gene. The replaced, inserted or modified SIRPα gene at the endogenous non-human animal (e.g., rodent) Sirpa locus can be detected using a variety of methods including, for example, PCR, Western blot, Southern blot, restriction fragment length polymorphism (RFLP), or a gain or loss of allele assay. In some embodiments, the non-human animal is heterozygous with respect to the humanized Sirpa gene.

In various embodiments, a humanized Sirpa gene according to the present disclosure includes a SIRPα gene that has a second, third and fourth exon each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a second, third and fourth exon that appear in a human SIRPA gene.

In various embodiments, a humanized Sirpa gene according to the present disclosure includes a SIRPα gene that has a nucleotide coding sequence (e.g., a cDNA sequence) at least 50% (e.g., 50%, 55%, 60%, 65%, 70%, 75%, 80%, 85%, 90%, 91%, 92%, 93%, 94%, 95%, 96%, 97%, 98%, 99% or more) identical to nucleotides 352-1114 that appear in a human SIRPA cDNA sequence.

In various embodiments, a humanized Sirpa protein produced by a non-human animal of the present disclosure has an extracellular portion having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an extracellular portion of a human SIRPA protein.

In various embodiments, a humanized Sirpa a protein produced by a non-human animal of the present disclosure has an extracellular portion having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to amino acid residues 28-362 that appear in a human SIRPA protein.

In various embodiments, a humanized Sirpa protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an amino acid sequence of a humanized SIRPA protein that appears in Table 3.

Compositions and methods for making non-human animals that express a humanized Sirpa protein, including specific polymorphic forms or allelic variants (e.g., single amino acid differences), are provided, including compositions and methods for making non-human animals that express such proteins from a human promoter and a human regulatory sequence. In some embodiments, compositions and methods for making non-human animals that express such proteins from an endogenous promoter and an endogenous regulatory sequence are also provided. The methods include inserting the genetic material encoding a human SIRPA protein in whole or in part at a precise location in the genome of a non-human animal that corresponds to an endogenous non-human animal (e.g., rodent) Sirpa gene thereby creating a humanized Sirpa gene that expresses a SIRPA protein that is human in whole or in part. In some embodiments, the methods include inserting genomic DNA corresponding to exons 2-4 of a human SIRPA gene into an endogenous non-human animal (e.g. rodent) Sirpa gene of the non-human animal thereby creating a humanized gene that encodes a Sirpa protein that contains a human portion containing amino acids encoded by the inserted exons.

In various embodiments, a humanized Sirpa gene approach employs a relatively minimal modification of the endogenous gene and results in natural Sirpa-mediated signal transduction in the non-human animal. Thus, in such embodiments, the Sirpa gene modification does not affect other surrounding genes or other endogenous non-human animal (e.g., rodent) Sirp genes. Further, in various embodiments, the modification does not affect the assembly of a functional receptor on the plasma and maintains normal effector functions via binding and subsequent signal transduction through the cytoplasmic portion of the receptor which is unaffected by the modification.

In addition to mice having humanized Sirpa genes as described herein, also provided herein are other genetically modified non-human animals (e.g., rodents, e.g., rats) that comprise humanized Sirpa genes. In some embodiments, such non-human animals comprise a humanized Sirpa gene operably linked to an endogenous Sirpa promoter. In some embodiments, such non-human animals express a humanized Sirpa protein from an endogenous locus, wherein the humanized Sirpa protein comprises amino acid residues 28-362 of a human SIRPA protein.

Humanized Sirpa polypeptides, loci encoding humanized Sirpa polypeptides and non-human animals expressing humanized Sirpa polypeptides are described in U.S. Pat. No. 11,019,810, WO 2014/039782, WO 2014/071397, and WO 2016/168212, each of which is incorporated by reference herein in its entirety.

### Humanized CD47 Loci

In one aspect, non-human animals are provided that are genetically modified to express one or more human proteins from their genome. In certain aspects, the genetically modified non-human animals provided herein further express a human or humanized CD47 protein encoded by a nucleic acid operably linked to a CD47 promoter.

CD47, originally named integrin-associated protein (IAP) for its role in signal transduction from integrins on immune cells, is a transmembrane protein that includes an N-terminal immunoglobulin V (IgV) domain, five transmembrane domains, and a short C-terminal intracytoplasmic tail. The intracytoplasmic tail differs in length according to four alternatively spliced isoforms that have been identified. CD47 (or IAP) was initially described as being expressed on all tissues (isoform 2), neurons (isoform 4) and keratinocytes and macrophages (isoform 1; see Reinhold et al. (1995) J. Cell Sci. 108:3419-3425). In addition to integrins, CD47 is known to interact with several other cell surface proteins such as, for example, thrombospondin and members of the SIRP family Most notably, CD47 interacts with SIRPA and leads to bidirectional signaling that regulates a variety of cell-to-cell responses such as, for example, inhibition of phagocytosis and T cell activation. Indeed, CD47-SIRPA interaction has come into focus in recent years for its role in providing tumor cells with the capacity to evade immune surveillance. CD47 binding to SIRPA normally provides protection through anti-phagocytic signals (“don't eat me”) for normal cells. However, it has been discovered that tumors also express anti-phagocytic signals, including CD47, to evade destruction by phagocytosis. Interestingly, CD47 is known to be upregulated in several hematologic cancers and contribute to both the growth and dissemination of tumors (Chao et al. (2012) Curr Opin Immunol. 24(2): 225-232).

Polypeptide sequence for wild-type human CD47 and the nucleic acid sequence that encode wild-type human CD47 may be found at Genbank Accession Nos. NP_001369235.1 and NM_001382306.1 (isoform 3 and transcript variant 3); NP_001768.1 and NM_001777.4 (isoform 1 and transcript variant 1); NP_942088.1 and NM_198793.3 (isoform 2 and transcript variant 2); and XP_005247966.1 and XM_005247909.3 (isoform X1 and transcript variant X1). The CD47 gene is conserved in at least chimpanzee, Rhesus monkey, dog, cow, mouse, rat, and chicken. The genomic locus encoding the wild-type human CD47 protein may be found in the human genome at Chromosome 3; NC_000003.12 (c108091031-108043091). In some embodiments, human CD47 protein is encoded by exons 1 through 11 at this locus. As such, in some embodiments, a nucleic acid sequence including coding sequence for human CD47 includes one or more of exons 1-11 of the human CD47 gene. In some instances, the nucleic acid sequence also includes aspects of the genomic locus of the human CD47, e.g., introns, 3′ and/or 5′ untranslated sequence (UTRs). In some instances, the nucleic acid sequence includes whole regions of the human CD47 genomic locus. In some instances, the nucleic acid sequence includes exons 2-7 of the human CD47 genomic locus.

Exemplary humanized CD47 sequences are set forth in Table 4. For humanized protein sequences, non-human (e.g., mouse) sequences are indicated in regular font, human sequences are indicated in bold font, and signal peptides are underlined. Representative mouse CD47 cDNA, mouse CD47 protein, human CD47 cDNA, and human CD47 protein sequences are described in U.S. Pat. Pub. No. 2021/0161112 A1, which is incorporated by reference herein in its entirety.

TABLE 4
Humanized CD47 amino acid isoform 1 (SEQ ID NO: 2)
MWPLAAALLLGSCCCGSAQLLENKTKSVEFTFCNDTVVIPCFVTNMEAQ
NTTEVYVKWKFKGRDIYTFDGALNKSTVPTDESSAKIEVSQLLKGDASL
KMDKSDAVSHTGNYTCEVTELTREGETIIELKYRVVSWFSPNENILIVI
FPIFAILLEWGQFGIKTLKYRSGGMDEKTIALLVAGLVITVIVIVGAIL
FVPGEYSLKNATGLGLIVTSTGILILLHYYVFSTAIGLTSFVIAILVIQ
VIAYILAVVGLSLCIAACIPMHGPLLISGLSILALAQLLGLVYMKFVE
Humanized CD47 amino acid isoform 2 (SEQ ID NO: 3)
MWPLAAALLLGSCCCGSAQLLENKTKSVEFTFCNDTVVIPCFVTNMEAQ
NTTEVYVKWKFKGRDIYTFDGALNKSTVPTDESSAKIEVSQLLKGDASL
KMDKSDAVSHTGNYTCEVTELTREGETIIELKYRVVSWFSPNENILIVI
FPIFAILLEWGQFGIKTLKYRSGGMDEKTIALLVAGLVITVIVIVGAIL
FVPGEYSLKNATGLGLIVTSTGILILLHYYVFSTAIGLTSFVIAILVIQ
VIAYILAVVGLSLCIAACIPMHGPLLISGLSILALAQLLGLVYMKFVAS
NQRTIQPPRNR
Humanized CD47 amino acid isoform 3 (SEQ ID NO: 4)
MWPLAAALLLGSCCCGSAQLLENKTKSVEFTFCNDTVVIPCFVTNMEAQ
NTTEVYVKWKFKGRDIYTFDGALNKSTVPTDFSSAKIEVSQLLKGDASL
KMDKSDAVSHTGNYTCEVTELTREGETIIELKYRVVSWESPNENILIVI
FPIFAILLEWGQFGIKTLKYRSGGMDEKTIALLVAGLVITVIVIVGAIL
FVPGEYSLKNATGLGLIVTSTGILILLHYYVFSTAIGLTSFVIAILVIQ
VIAYILAVVGLSLCIAACIPMHGPLLISGLSILALAQLLGLVYMKFVAS
NQRTIQPPRKAVEEPLNE
Humanized CD47 amino acid isoform 4 (SEQ ID NO: 5)
MWPLAAALLLGSCCCGSAQLLENKTKSVEFTFCNDTVVIPCFVTNMEAQ
NTTEVYVKWKFKGRDIYTFDGALNKSTVPTDESSAKIEVSQLLKGDASL
KMDKSDAVSHTGNYTCEVTELTREGETIIELKYRVVSWFSPNENILIVI
FPIFAILLEWGQFGIKTLKYRSGGMDEKTIALLVAGLVITVIVIVGAIL
FVPGEYSLKNATGLGLIVTSTGILILLHYYVFSTAIGLTSFVIAILVIQ
VIAYILAVVGLSLCIAACIPMHGPLLISGLSILALAQLLGLVYMKEVAS
NQRTIQPPRKAVEEPLNAFKESKGMMNDE

In some embodiments, the non-human animals provided herein express humanized CD47 proteins on the surface of cells of the non-human animals resulting from a genetic modification of an endogenous locus of the non-human animal that encodes a CD47 protein. Suitable examples described herein include rodents, for example, mice.

A humanized CD47 gene, in some embodiments, comprises genetic material from a heterologous species (e.g., humans), wherein the humanized CD47 gene encodes a CD47 protein that comprises the encoded portion of the genetic material from the heterologous species. In some embodiments, a humanized CD47 gene of the present disclosure comprises genomic DNA of a heterologous species that encodes the extracellular portion of a CD47 protein that is expressed on the plasma membrane of a cell. In some embodiments, a humanized CD47 gene of the present disclosure comprises genomic DNA of a heterologous species that encodes the extracellular portion and the transmembrane portion of a CD47 protein that is expressed on the plasma membrane of a cell. Non-human animals, embryos, cells and targeting constructs for making non-human animals, non-human embryos, and cells containing said humanized CD47 gene are also provided.

In some embodiments, an endogenous CD47 gene is deleted. In some embodiments, an endogenous CD47 gene is altered, wherein a portion of the endogenous CD47 gene is replaced with a heterologous sequence (e.g., a human CD47 sequence, in whole or in part). In some embodiments, all or substantially all of an endogenous CD47 gene is replaced with a heterologous gene (e.g., a human CD47 gene). In some embodiments, a portion of a heterologous CD47 gene is inserted into an endogenous non-human CD47 gene at an endogenous CD47 locus. In some embodiments, the heterologous gene is a human gene. In some embodiments, the modification or humanization is made to one of the two copies of the endogenous CD47 gene, giving rise to a non-human animal that is heterozygous with respect to the humanized CD47 gene. In other embodiments, a non-human animal is provided that is homozygous for a humanized CD47 gene.

In some embodiments, a non-human animal of the present disclosure contains a human CD47 gene, in whole or in part, at an endogenous non-human CD47 locus. Thus, such non-human animals can be described as having a heterologous CD47 gene. The replaced, inserted, modified or altered CD47 gene at the endogenous CD47 locus can be detected using a variety of methods including, for example, PCR, Western blot, Southern blot, restriction fragment length polymorphism (RFLP), or a gain or loss of allele assay. In some embodiments, the non-human animal is heterozygous with respect to the humanized CD47 gene. In some embodiments, the non-human animal is homozygous for the humanized CD47 gene.

In various embodiments, a humanized CD47 gene according to the present disclosure includes a CD47 gene that has a second, third, fourth, fifth, sixth and seventh exon each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a second, third, fourth, fifth, sixth and seventh exon that appear in a human CD47 gene.

In various embodiments, a humanized CD47 gene according to the present disclosure includes a CD47 gene that has a first exon and exon(s) downstream of exon 7 (e.g., eighth and ninth exons of isoform 2) each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a respective exon that appears in a mouse CD47 gene.

In various embodiments, a humanized CD47 gene according to the present disclosure includes a CD47 gene that has a 5′ untranslated region and a 3′ untranslated region each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a 5′ untranslated region and a 3′ untranslated region that appear in a mouse CD47 gene.

In various embodiments, a humanized CD47 gene according to the present disclosure includes a CD47 gene that has a nucleotide coding sequence (e.g., a cDNA sequence) at least 50% (e.g., 50%, 55%, 60%, 65%, 70%, 75%, 80%, 85%, 90%, 91%, 92%, 93%, 94%, 95%, 96%, 97%, 98%, 99% or more) identical to a nucleotide coding sequence that appears in a human CD47 nucleotide coding sequence.

In various embodiments, a humanized CD47 protein produced by a non-human animal of the present disclosure has an extracellular portion having an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an extracellular portion of a human CD47 protein.

In various embodiments, a humanized CD47 protein produced by a non-human animal of the present disclosure has an extracellular portion having an amino acid sequence that is identical to amino acid residues 19-141 that appear in a human CD47 protein.

In various embodiments, a humanized CD47 protein produced by a non-human animal of the present disclosure has an N-terminal immunoglobulin V domain having an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an N-terminal immunoglobulin V domain of a human CD47 protein.

In various embodiments, a humanized CD47 protein produced by a non-human animal of the present disclosure has an N-terminal immunoglobulin V domain having an amino acid sequence that is identical to amino acid residues 19-127 that appear in a human CD47 protein.

In various embodiments, a humanized CD47 protein produced by a non-human animal of the present disclosure has an N-terminal immunoglobulin V domain and five transmembrane domains each having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an N-terminal immunoglobulin V domain and five transmembrane domains of a human CD47 protein.

In various embodiments, a humanized CD47 protein produced by a non-human animal of the present disclosure has an intracytoplasmic tail having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an intracytoplasmic tail of a mouse CD47 protein.

In various embodiments, a humanized CD47 protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to amino acid residues 16-292 that appear in a human CD47 protein.

In various embodiments, a humanized CD47 protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to amino acid residues 19-292 that appear in a human CD47 protein.

In various embodiments, a humanized CD47 protein produced by a non-human animal of the present disclosure has an amino acid sequence that is identical to amino acid residues 19-292 (or 16-292) that appear in a human CD47 protein.

In various embodiments, a humanized CD47 protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an amino acid sequence of a humanized CD47 protein that appears in Table 4.

In various embodiments, a humanized CD47 protein produced by a non-human animal of the present disclosure has an amino acid sequence that is identical to an amino acid sequence of a humanized CD47 protein that appears in Table 4.

Compositions and methods for making non-human animals that express a humanized CD47 protein, including specific polymorphic forms, allelic variants (e.g., single amino acid differences) or alternatively spliced isoforms, are provided, including compositions and methods for making non-human animals that express such proteins from a human promoter and a human regulatory sequence. In some embodiments, compositions and methods for making non-human animals that express such proteins from an endogenous promoter and an endogenous regulatory sequence are also provided. The methods include inserting the genetic material encoding a human CD47 protein in whole or in part at a precise location in the genome of a non-human animal that corresponds to an endogenous CD47 gene thereby creating a humanized CD47 gene that expresses a CD47 protein that is human in whole or in part. In some embodiments, the methods include inserting genomic DNA corresponding to exons 2-7 of a human CD47 gene into an endogenous CD47 gene of the non-human animal thereby creating a humanized gene that encodes a CD47 protein that contains a human portion containing amino acids encoded by the inserted exons.

Where appropriate, the coding region of the genetic material or polynucleotide sequence(s) encoding a human CD47 protein in whole or in part may be modified to include codons that are optimized for expression in the non-human animal (e.g., see U.S. Pat. Nos. 5,670,356 and 5,874,304). Codon optimized sequences are synthetic sequences, and preferably encode the identical polypeptide (or a biologically active fragment of a full-length polypeptide which has substantially the same activity as the full-length polypeptide) encoded by the non-codon optimized parent polynucleotide. In some embodiments, the coding region of the genetic material encoding a human CD47 protein, in whole or in part, may include an altered sequence to optimize codon usage for a particular cell type (e.g., a rodent cell). For example, the codons of the genomic DNA corresponding to exons 2-7 of a human CD47 gene to be inserted into an endogenous CD47 gene of a non-human animal (e.g., a rodent) may be optimized for expression in a cell of the non-human animal. Such a sequence may be described as a codon-optimized sequence.

A humanized CD47 gene approach employs a relatively minimal modification of the endogenous gene and results in natural CD47-mediated signal transduction in the non-human animal, in various embodiments, because the genomic sequence of the CD47 sequences are modified in a single fragment and therefore retain normal functionality by including necessary regulatory sequences. Thus, in such embodiments, the CD47 gene modification does not affect other surrounding genes or other endogenous CD47-interacting genes (e.g., thrombospondin, SIRPs, integrins, etc.). Further, in various embodiments, the modification does not affect the assembly of a functional CD47 transmembrane protein on the plasma membrane and maintains normal effector functions via binding and subsequent signal transduction through the cytoplasmic portion of the protein which is unaffected by the modification.

Although embodiments employing a humanized CD47 gene in a mouse (i.e., a mouse with a CD47 gene that encodes a CD47 protein that includes a human portion and a mouse portion) are extensively discussed herein, other non-human animals (e.g., rodents, e.g., rats) that comprise a humanized CD47 gene are also provided. In some embodiments, such non-human animals comprise a humanized CD47 gene operably linked to an endogenous CD47 promoter. In some embodiments, such non-human animals express a humanized CD47 protein from an endogenous locus, wherein the humanized CD47 protein comprises amino acid residues 16-292 (or 19-141 or 19-127) of a human CD47 protein.

Humanized CD47 polypeptides, loci encoding humanized CD47 polypeptides and non-human animals expressing humanized CD47 polypeptides are described in U.S. Pat. Publication No. 2021/0161112, which is incorporated by reference herein.

### Humanized M-CSF Loci

In some aspects, the genetically modified non-human animals provided herein further express a human M-CSF protein encoded by a nucleic acid operably linked to an M-CSF promoter. By a human M-CSF protein, it is a meant a protein that is human M-CSF or is substantially identical to human M-CSF, e.g., it is 80% or more identical, 85% or more identical, 90% or more identical, or 95% or more identical to human M-CSF, for example, 97%, 98%, or 99% identical to human M-CSF. A nucleic acid sequence that encodes a human M-CSF protein is, therefore, a polynucleotide that comprises coding sequence for a human M-CSF protein, i.e., human M-CSF or a protein that is substantially identical to human M-CSF.

M-CSF (also known as CSF-1, for “colony stimulating factor 1”) is a cytokine that controls the production, differentiation, and function of macrophages. Polypeptide sequence for human M-CSF and the nucleic acid sequence that encodes for human M-CSF may be found at Genbank Accession Nos. NP_000748.4 and NM_000757.6 (isoform a and transcript variant 1); NP_757349.2 and NM_172210.3 (isoform b and transcript variant 2); NP_757350.2 and NM_172211.4 (isoform c and transcript variant 3) and NP_757351.2; and NM 172212.3 (isoform a and transcript variant 4). The genomic locus encoding the human M-CSF protein may be found in the human genome at Chromosome 1; NC_000001.11 (109910506-109930992). Protein sequence is encoded by exons 1 through 8 at this locus, while exon 9 comprises untranslated sequence. As such, a nucleic acid sequence comprising coding sequence for human M-CSF comprises one or more of exons 1-8 of the human M-CSF gene. In some instances, the nucleic acid sequence also comprises aspects of the genomic locus of the human M-CSF, e.g., introns, 3′ and/or 5′ untranslated sequence (UTRs). In some instances, the nucleic acid sequence comprises whole regions of the human M-CSF genomic locus. In some instances, the nucleic acid sequence comprises exon 2 of the human M-CSF genomic locus to 633 nucleotides downstream of noncoding exon 9.

In some embodiments, in the genetically modified non-human animals provided herein, the nucleic acid sequence that encodes a human M-CSF protein is operably linked to one or more regulatory sequences of the non-human animal (e.g., mouse) M-CSF gene. Non-human animal (e.g., mouse) M-CSF regulatory sequences are those sequences of the non-human animal (e.g., mouse) M-CSF genomic locus that regulate non-human animal (e.g., mouse) M-CSF expression, for example, 5′ regulatory sequences, e.g., the M-CSF promoter, M-CSF 5′ untranslated region (UTR), etc.; 3′ regulatory sequences, e.g., the 3′UTR; and enhancers, etc. For example, mouse M-CSF is located on chromosome 3, NC_000069.7, at about positions c107668048-107648364, and the mouse M-CSF coding sequence may be found at Genbank Accession Nos. NM_007778.4 (transcript variant 1 encoding isoform 1), NM_001113529.1 (transcript variant 2 encoding isoform 2), and NM 001113530.1 (transcript variant 3 encoding isoform 1). The regulatory sequences of mouse M-CSF are well defined in the art, and may be readily identified using in silico methods, e.g., by referring to the above Genbank Accession Nos. on the UCSC Genome Browser, on the world wide web at genome.ucsc.edu, or by experimental methods as described in the art, e.g., Abboud et al. (2003) Analysis of the Mouse CSF-1 Gene Promoter in a Transgenic Mouse Model. J. Histochemistry and Cytochemistry 51 (7):941-949, the disclosure of which is incorporated herein by reference. In some instances, e.g., when the nucleic acid sequence that encodes a human M-CSF protein is located at the non-human animal (e.g., mouse) M-CSF genomic locus, the regulatory sequences operably linked to the human CSF coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human M-CSF protein is generated by the random integration, or insertion, of human nucleic acid sequence encoding human M-CSF protein or a fragment thereof, i.e., “human M-CSF nucleic acid sequence”, or “human M-CSF sequence”, into the genome of the non-human animal. Typically, in such embodiments, the location of the nucleic acid sequence encoding a human M-CSF protein in the genome is unknown. In other instances, the genetically modified non-human animal expressing a human M-CSF protein is generated by the targeted integration, or insertion, of human M-CSF nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human M-CSF protein created by targeting human M-CSF nucleic acid sequence to the non-human animal M-CSF (e.g., mouse) locus, human M-CSF nucleic acid sequence may replace some or all of the non-human animal (e.g., mouse) sequence, e.g., exons and/or introns, at the M-CSF locus. In some such instances, human M-CSF nucleic acid sequence is integrated into the non-human animal (e.g., mouse) M-CSF locus such that expression of the human M-CSF sequence is regulated by the native, or endogenous, regulatory sequences at the non-human animal (e.g., mouse) M-CSF locus. In other words, the regulatory sequence(s) to which the nucleic acid sequence encoding a human M-CSF protein is operably linked are the native M-CSF regulatory sequences at the non-human animal (e.g., mouse) M-CSF locus.

In some instances, the integration of human M-CSF sequence does not affect the transcription of the gene into which the human M-CSF sequence has integrated. For example, if the human M-CSF sequence integrates into coding sequence as an intein, or the human M-CSF sequence comprises a 2A peptide, the human M-CSF sequence will be transcribed and translated simultaneously with the gene into which the human M-CSF sequence has integrated. In other instances, the integration of the human M-CSF sequence interrupts the transcription of the gene into which the human M-CSF sequence has integrated. For example, upon integration of the human M-CSF sequence by homologous recombination, some or all of the coding sequence at the integration locus may be removed, such that the human M-CSF sequence is transcribed instead. In some such instances, the integration of human M-CSF sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the genetically modified non-human animal (e.g., mouse) expressing a human M-CSF protein comprises one copy of the nucleic acid sequence encoding a human M-CSF protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human M-CSF nucleic acid sequence is integrated into the non-human animal (e.g., mouse) M-CSF locus such that it creates a null allele for non-human animal (e.g., mouse) M-CSF. In some such embodiments, the humanized M-CSF mouse may be heterozygous for the nucleic acid sequence encoding, i.e., the humanized M-CSF mouse comprises one null allele for non-human animal (e.g., mouse) M-CSF (the allele comprising the nucleic acid sequence) and one endogenous M-CSF allele (wild type or otherwise). In other instances, the genetically modified non-human animal (e.g., mouse) expressing a human M-CSF protein comprises two copies of the nucleic acid sequence encoding a human M-CSF protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human M-CSF protein comprises two null alleles for the mouse M-CSF (the allele comprising the nucleic acid sequence).

Although embodiments employing a human M-CSF gene in a mouse are extensively discussed herein, other non-human animals (e.g., rodents, e.g., rats) that comprise a a human M-CSF gene are also provided.

Human M-CSF polypeptides, loci encoding human M-CSF polypeptides and non-human animals expressing human M-CSF polypeptides are described in WO 2012/112544, WO 2014/039782, and WO 2014/071397, each of which is incorporated by reference herein.

### Humanized GM-CSF Loci

In some aspects, the genetically modified non-human animals provided herein further express a human GM-CSF protein encoded by a nucleic acid operably linked to a GM-CSF promoter. By a human GM-CSF protein, it is a meant a protein that is human GM-CSF or is substantially identical to human GM-CSF, e.g., it is 80% or more identical, 85% or more identical, 90% or more identical, or 95% or more identical to human GM-CSF, for example, 97%, 98%, or 99% identical to human GM-CSF. A nucleic acid sequence that encodes a human GM-CSF protein is, therefore, a polynucleotide that comprises coding sequence for a human GM-CSF protein, i.e., human GM-CSF or a protein that is substantially identical to human GM-CSF.

GM-CSF is a cytokine crucial for myeloid cell development and function. GM-CSF is not cross-reactive between human and mouse. GM-CSF is highly expressed in the lung and important for lung homeostasis in vivo, as demonstrated by the fact that GM-CSF KO mice develop pulmonary alveolar proteinosis (PAP) which is characterized by protein accumulation in the lung due to defective surfactant clearance. Alveolar macrophages from GM-CSF KO mice have a defect in terminal differentiation, which leads to impaired innate immunity to pathogens in the lung. GM-CSF also stimulates the proliferation of human alveolar macrophages (AM) in vitro. GM-CSF is largely dispensable for steady-state hematopoiesis. In contrast, GM-CSF is required for inflammatory responses such as the production of proinflammatory cytokines by macrophages and the mobilization and recruitment of monocytes. GM-CSF is also essential for protective immunity against a range of pathogens, including M. tuberculosis. In particular, GM-CSF KO mice infected with M. tuberculosis do not develop granulomas, a hallmark of tuberculosis.

Polypeptide sequence for human GM-CSF and the nucleic acid sequence that encodes for human GM-CSF may be found at Genbank Accession Nos. NP_000749.2 and NM_000758.4, respectively. The genomic locus encoding the human GM-CSF protein may be found in the human genome at Chromosome 5; NG_033024.1 (4998-7379). Protein sequence is encoded by exons 1 through 4 at this locus. As such, a nucleic acid sequence comprising coding sequence for human GM-CSF comprises one or more of exons 1-4 of the human GM-CSF gene. In some instances, the nucleic acid sequence also comprises aspects of the genomic locus of the human GM-CSF, e.g., introns, 3′ and/or 5′ untranslated sequence (UTRs). In some instances, the nucleic acid sequence comprises whole regions of the human GM-CSF genomic locus.

In some embodiments, in the genetically modified non-human animals provided herein, the nucleic acid sequence that encodes a human GM-CSF protein is operably linked to one or more regulatory sequences of the non-human animal (e.g., mouse) GM-CSF gene. Non-human animal (e.g., mouse) GM-CSF regulatory sequences are those sequences of the non-human animal (e.g., mouse) GM-CSF genomic locus that regulate non-human animal (e.g., mouse) GM-CSF expression, for example, 5′ regulatory sequences, e.g., the GM-CSF promoter, GM-CSF 5′ untranslated region (UTR), etc.; 3′ regulatory sequences, e.g., the 3′UTR; and enhancers, etc. For example, mouse GM-CSF is located on chromosome 11, GRCm39, NC_000077.7, at about positions c54140725-54138096, and the mouse GM-CSF coding sequence may be found at Genbank Accession No. NM_009969.4. The regulatory sequences of mouse GM-CSF are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human GM-CSF protein is located at the non-human animal (e.g., mouse) GM-CSF genomic locus, the regulatory sequences operably linked to the human GM-CSF coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human GM-CSF protein is generated by the random integration, or insertion, of human nucleic acid sequence encoding human GM-CSF protein or a fragment thereof, i.e., “human GM-CSF nucleic acid sequence”, or “human GM-CSF sequence”, into the genome of the non-human animal. Typically, in such embodiments, the location of the nucleic acid sequence encoding a human GM-CSF protein in the genome is unknown. In other instances, the genetically modified non-human animal expressing a human GM-CSF protein is generated by the targeted integration, or insertion, of human GM-CSF nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human GM-CSF protein created by targeting human GM-CSF nucleic acid sequence to the non-human animal GM-CSF (e.g., mouse) locus, human GM-CSF nucleic acid sequence may replace some or all of the non-human animal (e.g., mouse) sequence, e.g., exons and/or introns, at the GM-CSF locus. In some such instances, human GM-CSF nucleic acid sequence is integrated into the non-human animal (e.g., mouse) GM-CSF locus such that expression of the human GM-CSF sequence is regulated by the native, or endogenous, regulatory sequences at the non-human animal (e.g., mouse) GM-CSF locus. In other words, the regulatory sequence(s) to which the nucleic acid sequence encoding a human GM-CSF protein is operably linked are the native GM-CSF regulatory sequences at the non-human animal (e.g., mouse) GM-CSF locus.

In some instances, the integration of human GM-CSF sequence does not affect the transcription of the gene into which the human GM-CSF sequence has integrated. For example, if the human GM-CSF sequence integrates into coding sequence as an intein, or the human GM-CSF sequence comprises a 2A peptide, the human GM-CSF sequence will be transcribed and translated simultaneously with the gene into which the human GM-CSF sequence has integrated. In other instances, the integration of the human GM-CSF sequence interrupts the transcription of the gene into which the human GM-CSF sequence has integrated. For example, upon integration of the human GM-CSF sequence by homologous recombination, some or all of the coding sequence at the integration locus may be removed, such that the human GM-CSF sequence is transcribed instead. In some such instances, the integration of human GM-CSF sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the genetically modified non-human animal (e.g., mouse) expressing a human GM-CSF protein comprises one copy of the nucleic acid sequence encoding a human GM-CSF protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human GM-CSF nucleic acid sequence is integrated into the non-human animal (e.g., mouse) GM-CSF locus such that it creates a null allele for non-human animal (e.g., mouse) GM-CSF. In some such embodiments, the humanized GM-CSF mouse may be heterozygous for the nucleic acid sequence encoding, i.e., the humanized GM-CSF mouse comprises one null allele for non-human animal (e.g., mouse) GM-CSF (the allele comprising the nucleic acid sequence) and one endogenous GM-CSF allele (wild type or otherwise). In other instances, the genetically modified non-human animal (e.g., mouse) expressing a human GM-CSF protein comprises two copies of the nucleic acid sequence encoding a human GM-CSF protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human GM-CSF protein comprises two null alleles for the mouse GM-CSF (the allele comprising the nucleic acid sequence).

Although embodiments employing a human GM-CSF gene in a mouse are extensively discussed herein, other non-human animals (e.g., rodents, e.g., rats) that comprise a a human GM-CSF gene are also provided.

Human GM-CSF polypeptides, loci encoding human GM-CSF polypeptides and non-human animals expressing human GM-CSF polypeptides are described in WO2011/044050, WO 2014/039782 and WO 2014/071397, each of which is incorporated by reference herein.

### Humanized TPO Loci

In some aspects, the genetically modified non-human animals provided herein further express a human TPO protein encoded by a nucleic acid operably linked to a TPO promoter. By a human TPO protein, it is a meant a protein that is human TPO or is substantially identical to human TPO, e.g., it is 80% or more identical, 85% or more identical, 90% or more identical, or 95% or more identical to human TPO, for example, 97%, 98%, or 99% identical to human TPO. A nucleic acid sequence that encodes a human TPO protein is, therefore, a polynucleotide that comprises coding sequence for a human TPO protein, i.e., human TPO or a protein that is substantially identical to human TPO.

Thrombopoietin (TPO) was initially identified as a growth factor that promotes the development of megakaryocytes and platelets. TPO is constitutively produced by the liver and the kidneys and released into the blood circulation. The receptor for TPO, c-Mpl, is expressed by hematopoietic stem and progenitor cells in the bone marrow. C-Mpl is also expressed on circulating platelets. However, the binding of TPO on platelets does not activate any signaling pathway. Thus, thrombocytes act as a sink or scavengers for TPO and via this mechanism contribute to negative regulation of thrombopoiesis. Subsequently, TPO has been recognized for its important function to support the expansion and self-renewal of HSCs. TPO deficiency leads to reduced numbers of HSCs in adult mice, and the presence of TPO is needed to maintain adult HSCs in quiescence. Furthermore, TPO is required to support post-transplantation expansion of HSCs, necessary to replenish the hematopoietic compartment of irradiated hosts. Interestingly, it has been demonstrated that osteoblastic cells involved in forming the HSC niche in the bone marrow produce TPO, critical for HSC function and maintenance.

Polypeptide sequence for human TPO and the nucleic acid sequence that encodes for human TPO may be found at Genbank Accession Nos. NM_000547.6 and NP_000538.3 (transcript variant 1 and isoform a); NM_001206744.2 and NP_001193673.1 (transcript variant 6 and isoform a); NM_001206745.2 and NP_001193674.1 (transcript variant 7 and isoform b); NM_175719.4 and NP_783650.1 (transcript variant 2 and isoform b); NM_175721.3 and NP_783652.1 (transcript variant 4 and isoform d); and NM_175722.3 and NP_783653.1 (transcript variant 5 and isoform e). The genomic locus encoding the human TPO protein may be found in the human genome at Chromosome 2; NG_011581.1 (4999-134265). Protein sequence is encoded by exons 2 through 17 at this locus. As such, a nucleic acid sequence comprising coding sequence for human TPO comprises one or more of exons 2-17 of the human TPO gene. In some instances, the nucleic acid sequence also comprises aspects of the genomic locus of the human TPO, e.g., introns, 3′ and/or 5′ untranslated sequence (UTRs). In some instances, the nucleic acid sequence comprises whole regions of the human TPO genomic locus.

In some embodiments, in the genetically modified non-human animals provided herein, the nucleic acid sequence that encodes a human TPO protein is operably linked to one or more regulatory sequences of the non-human animal (e.g., mouse) TPO gene. Non-human animal (e.g., mouse) TPO regulatory sequences are those sequences of the non-human animal (e.g., mouse) TPO genomic locus that regulate non-human animal (e.g., mouse) TPO expression, for example, 5′ regulatory sequences, e.g., the TPO promoter, TPO 5′ untranslated region (UTR), etc.; 3′ regulatory sequences, e.g., the 3′UTR; and enhancers, etc. For example, mouse TPO is located on chromosome 12, GRCm39, NC_000078.7, at about positions c30182983-30104658, and the mouse TPO coding sequence may be found at Genbank Accession No. NM_009417.3. The regulatory sequences of mouse TPO are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human TPO protein is located at the non-human animal (e.g., mouse) TPO genomic locus, the regulatory sequences operably linked to the human TPO coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human TPO protein is generated by the random integration, or insertion, of human nucleic acid sequence encoding human TPO protein or a fragment thereof, i.e., “human TPO nucleic acid sequence”, or “human TPO sequence”, into the genome of the non-human animal. Typically, in such embodiments, the location of the nucleic acid sequence encoding a human TPO protein in the genome is unknown. In other instances, the genetically modified non-human animal expressing a human TPO protein is generated by the targeted integration, or insertion, of human TPO nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human TPO protein created by targeting human TPO nucleic acid sequence to the non-human animal TPO (e.g., mouse) locus, human TPO nucleic acid sequence may replace some or all of the non-human animal (e.g., mouse) sequence, e.g., exons and/or introns, at the TPO locus. In some such instances, human TPO nucleic acid sequence is integrated into the non-human animal (e.g., mouse) TPO locus such that expression of the human TPO sequence is regulated by the native, or endogenous, regulatory sequences at the non-human animal (e.g., mouse) TPO locus. In other words, the regulatory sequence(s) to which the nucleic acid sequence encoding a human TPO protein is operably linked are the native TPO regulatory sequences at the non-human animal (e.g., mouse) TPO locus.

In some instances, the integration of human TPO sequence does not affect the transcription of the gene into which the human TPO sequence has integrated. For example, if the human TPO sequence integrates into coding sequence as an intein, or the human TPO sequence comprises a 2A peptide, the human TPO sequence will be transcribed and translated simultaneously with the gene into which the human TPO sequence has integrated. In other instances, the integration of the human TPO sequence interrupts the transcription of the gene into which the human TPO sequence has integrated. For example, upon integration of the human TPO sequence by homologous recombination, some or all of the coding sequence at the integration locus may be removed, such that the human TPO sequence is transcribed instead. In some such instances, the integration of human TPO sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the genetically modified non-human animal (e.g., mouse) expressing a human TPO protein comprises one copy of the nucleic acid sequence encoding a human TPO protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human TPO nucleic acid sequence is integrated into the non-human animal (e.g., mouse) TPO locus such that it creates a null allele for non-human animal (e.g., mouse) TPO. In some such embodiments, the humanized TPO mouse may be heterozygous for the nucleic acid sequence encoding, i.e., the humanized TPO mouse comprises one null allele for non-human animal (e.g., mouse) TPO (the allele comprising the nucleic acid sequence) and one endogenous TPO allele (wild type or otherwise). In other instances, the genetically modified non-human animal (e.g., mouse) expressing a human TPO protein comprises two copies of the nucleic acid sequence encoding a human TPO protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human TPO protein comprises two null alleles for the mouse TPO (the allele comprising the nucleic acid sequence).

Although embodiments employing a human TPO gene in a mouse are extensively discussed herein, other non-human animals (e.g., rodents, e.g., rats) that comprise a a human TPO gene are also provided.

Human TPO polypeptides, loci encoding human TPO polypeptides and non-human animals expressing human TPO polypeptides are described in WO2011/044050, WO 2014/039782 and WO 2014/071397, each of which is incorporated by reference herein.

### Humanized EPO Loci

In some aspects, genetically modified non-human animals provided herein further express a human erythropoietin (hEPO) protein encoded by a nucleic acid operably linked to an EPO promoter. By a human EPO protein, it is a meant a protein that is human EPO or is substantially identical to human EPO, e.g., it is 80% or more identical, 85% or more identical, 90% or more identical, or 95% or more identical to human EPO, for example, 97%, 98%, or 99% identical to human EPO. A nucleic acid sequence that encodes a human EPO protein is, therefore, a polynucleotide that comprises coding sequence for a human EPO protein, i.e., human EPO or a protein that is substantially identical to human EPO.

Erythropoietin (EPO) encodes a secreted, glycosylated cytokine composed of four alpha helical bundles. The encoded EPO protein is mainly synthesized in the kidney, secreted into the blood plasma, and binds to the erythropoietin receptor to promote red blood cell production, or erythropoiesis, in the bone marrow. Expression of EPO gene is upregulated under hypoxic conditions, in turn leading to increased erythropoiesis and enhanced oxygen-carrying capacity of the blood. Expression of EPO gene has also been observed in brain and in the eye, and elevated expression levels have been observed in diabetic retinopathy and ocular hypertension. Recombinant forms of the encoded EPO protein exhibit neuroprotective activity against a variety of potential brain injuries, as well as antiapoptotic functions in several tissue types, and have been used in the treatment of anemia and to enhance the efficacy of cancer therapies.

Polypeptide sequence for human EPO and the nucleic acid sequence that encodes for human EPO may be found at Genbank Accession Nos. NP_000790.2 and NM_000799.4, respectively. The genomic locus encoding the human EPO protein may be found in the human genome at Chromosome 7, NG_021471.2 (4669-7901) or NC_000007.14 (100720468-100723700). Protein sequence is encoded by exons 1 through 5 at this locus. As such, a nucleic acid sequence comprising coding sequence for human EPO comprises one or more of exons 1-5 of the human EPO gene. In some instances, the nucleic acid sequence also comprises aspects of the genomic locus of the human EPO, e.g., introns, 3′ and/or 5′ untranslated sequence (UTRs). In some instances, the nucleic acid sequence comprises whole regions of the human EPO genomic locus.

In some embodiments, in the genetically modified non-human animals provided herein, the nucleic acid sequence that encodes a human EPO protein is operably linked to one or more regulatory sequences of the non-human animal (e.g., mouse) EPO gene. Non-human animal (e.g., mouse) EPO regulatory sequences are those sequences of the non-human animal (e.g., mouse) EPO genomic locus that regulate non-human animal (e.g., mouse) EPO expression, for example, 5′ regulatory sequences, e.g., the EPO promoter, EPO 5′ untranslated region (UTR), etc.; 3′ regulatory sequences, e.g., the 3′UTR; and enhancers, etc. For example, mouse EPO is located on chromosome 5, GRCm39, NC_000071.7, at about positions c137484078-137481282, and the mouse EPO coding sequence may be found at Genbank Accession Nos. NM_007942.2 (transcript variant 1 encoding isoform 1), and NM_001312875.1 (transcript variant 2 encoding isoform 2). The regulatory sequences of mouse EPO are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human EPO protein is located at the non-human animal (e.g., mouse) EPO genomic locus, the regulatory sequences operably linked to the human EPO coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human EPO protein is generated by the random integration, or insertion, of human nucleic acid sequence encoding human EPO protein or a fragment thereof, i.e., “human EPO nucleic acid sequence”, or “human EPO sequence”, into the genome of the non-human animal. Typically, in such embodiments, the location of the nucleic acid sequence encoding a human EPO protein in the genome is unknown. In other instances, the genetically modified non-human animal expressing a human EPO protein is generated by the targeted integration, or insertion, of human EPO nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human EPO protein created by targeting human EPO nucleic acid sequence to the non-human animal EPO (e.g., mouse) locus, human EPO nucleic acid sequence may replace some or all of the non-human animal (e.g., mouse) sequence, e.g., exons and/or introns, at the EPO locus. In some such instances, human EPO nucleic acid sequence is integrated into the non-human animal (e.g., mouse) EPO locus such that expression of the human EPO sequence is regulated by the native, or endogenous, regulatory sequences at the non-human animal (e.g., mouse) EPO locus. In other words, the regulatory sequence(s) to which the nucleic acid sequence encoding a human EPO protein is operably linked are the native EPO regulatory sequences at the non-human animal (e.g., mouse) EPO locus.

In some instances, the integration of human EPO sequence does not affect the transcription of the gene into which the human EPO sequence has integrated. For example, if the human EPO sequence integrates into coding sequence as an intein, or the human EPO sequence comprises a 2A peptide, the human EPO sequence will be transcribed and translated simultaneously with the gene into which the human EPO sequence has integrated. In other instances, the integration of the human EPO sequence interrupts the transcription of the gene into which the human EPO sequence has integrated. For example, upon integration of the human EPO sequence by homologous recombination, some or all of the coding sequence at the integration locus may be removed, such that the human EPO sequence is transcribed instead. In some such instances, the integration of human EPO sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the genetically modified non-human animal (e.g., mouse) expressing a human EPO protein comprises one copy of the nucleic acid sequence encoding a human EPO protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human EPO nucleic acid sequence is integrated into the non-human animal (e.g., mouse) EPO locus such that it creates a null allele for non-human animal (e.g., mouse) EPO. In some such embodiments, the humanized EPO mouse may be heterozygous for the nucleic acid sequence encoding, i.e., the humanized EPO mouse comprises one null allele for non-human animal (e.g., mouse) EPO (the allele comprising the nucleic acid sequence) and one endogenous EPO allele (wild type or otherwise). In other instances, the genetically modified non-human animal (e.g., mouse) expressing a human EPO protein comprises two copies of the nucleic acid sequence encoding a human EPO protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human EPO protein comprises two null alleles for the mouse EPO (the allele comprising the nucleic acid sequence).

Although embodiments employing a human EPO gene in a mouse are extensively discussed herein, other non-human animals (e.g., rodents, e.g., rats) that comprise a a human EPO gene are also provided.

Human EPO polypeptides, loci encoding human EPO polypeptides and non-human animals expressing human EPO polypeptides are described in WO 2015/179317, which is incorporated by reference herein.

### Humanized IL-3 Loci

In some aspects, the genetically modified non-human animals provided herein further express a human IL-3 protein encoded by a nucleic acid operably linked to an IL-3 promoter. By a human IL-3 protein, it is a meant a protein that is human IL-3 or is substantially identical to human IL-3, e.g., it is 80% or more identical, 85% or more identical, 90% or more identical, or 95% or more identical to human IL-3, for example, 97%, 98%, or 99% identical to human IL-3. A nucleic acid sequence that encodes a human IL-3protein is, therefore, a polynucleotide that comprises coding sequence for a human IL-3protein, i.e., human IL-3 or a protein that is substantially identical to human IL-3.

Like GM-CSF, IL-3 is a cytokine crucial for myeloid cell development and function. IL-3 is not cross-reactive between human and mouse. IL-3 stimulates early hematopoietic progenitors in vitro, but is dispensable for steady-state hematopoiesis in vivo. However, together with GM-CSF it is required for effective DTH responses in vivo. IL-3 also specifically stimulates the proliferation of alveolar macrophages (AM) in vitro.

Polypeptide sequence for human IL-3 and the nucleic acid sequence that encodes for human IL-3 may be found at Genbank Accession Nos. NP_000579.2 and NM_000588.4. The genomic locus encoding the human IL-3 protein may be found in the human genome at chromosome 5, GRCh38.p14; NC_000005.10 (132060655-132063204). Protein sequence is encoded by exons 1 through 5 at this locus. As such, a nucleic acid sequence comprising coding sequence for human IL-3 comprises one or more of exons 1-5 of the human IL-3 gene. In some instances, the nucleic acid sequence also comprises aspects of the genomic locus of the human IL-3, e.g., introns, 3′ and/or 5′ untranslated sequence (UTRs). In some instances, the nucleic acid sequence comprises whole regions of the human IL-3 genomic locus.

In some embodiments, in the genetically modified non-human animals provided herein, the nucleic acid sequence that encodes a human IL-3 protein is operably linked to one or more regulatory sequences of the non-human animal (e.g., mouse) IL-3 gene. Non-human animal (e.g., mouse) IL-3 regulatory sequences are those sequences of the non-human animal (e.g., mouse) IL-3 genomic locus that regulate non-human animal (e.g., mouse) IL-3 expression, for example, 5′ regulatory sequences, e.g., the IL-3 promoter, IL-3 5′ untranslated region (UTR), etc.; 3′ regulatory sequences, e.g., the 3′UTR; and enhancers, etc. For example, mouse IL-3 is located on chromosome 11, GRCm39, NC_000077.7, at about positions c54158105-54155911, and the mouse IL-3 coding sequence may be found at Genbank Accession No. NM_010556.4. The regulatory sequences of mouse IL-3 are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human IL-3 protein is located at the non-human animal (e.g., mouse) IL-3 genomic locus, the regulatory sequences operably linked to the human IL-3 coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human IL-3 protein is generated by the random integration, or insertion, of human nucleic acid sequence encoding human IL-3 protein or a fragment thereof, i.e., “human IL-3 nucleic acid sequence”, or “human IL-3 sequence”, into the genome of the non-human animal. Typically, in such embodiments, the location of the nucleic acid sequence encoding a human IL-3 protein in the genome is unknown. In other instances, the genetically modified non-human animal expressing a human IL-3 protein is generated by the targeted integration, or insertion, of human IL-3 nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human IL-3 protein created by targeting human IL-3 nucleic acid sequence to the non-human animal IL-3 (e.g., mouse) locus, human IL-3 nucleic acid sequence may replace some or all of the non-human animal (e.g., mouse) sequence, e.g., exons and/or introns, at the IL-3 locus. In some such instances, human IL-3 nucleic acid sequence is integrated into the non-human animal (e.g., mouse) IL-3 locus such that expression of the human IL-3 sequence is regulated by the native, or endogenous, regulatory sequences at the non-human animal (e.g., mouse) IL-3 locus. In other words, the regulatory sequence(s) to which the nucleic acid sequence encoding a human IL-3 protein is operably linked are the native IL-3 regulatory sequences at the non-human animal (e.g., mouse) IL-3 locus.

In some instances, the integration of human IL-3 sequence does not affect the transcription of the gene into which the human IL-3 sequence has integrated. For example, if the human IL-3 sequence integrates into coding sequence as an intein, or the human IL-3 sequence comprises a 2A peptide, the human IL-3 sequence will be transcribed and translated simultaneously with the gene into which the human IL-3 sequence has integrated. In other instances, the integration of the human IL-3 sequence interrupts the transcription of the gene into which the human IL-3 sequence has integrated. For example, upon integration of the human IL-3 sequence by homologous recombination, some or all of the coding sequence at the integration locus may be removed, such that the human IL-3 sequence is transcribed instead. In some such instances, the integration of human IL-3 sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the genetically modified non-human animal (e.g., mouse) expressing a human IL-3 protein comprises one copy of the nucleic acid sequence encoding a human IL-3 protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human IL-3 nucleic acid sequence is integrated into the non-human animal (e.g., mouse) IL-3 locus such that it creates a null allele for non-human animal (e.g., mouse) IL-3. In some such embodiments, the humanized IL-3 mouse may be heterozygous for the nucleic acid sequence encoding, i.e., the humanized IL-3 mouse comprises one null allele for non-human animal (e.g., mouse) IL-3 (the allele comprising the nucleic acid sequence) and one endogenous IL-3 allele (wild type or otherwise). In other instances, the genetically modified non-human animal (e.g., mouse) expressing a human IL-3 protein comprises two copies of the nucleic acid sequence encoding a human IL-3 protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human IL-3 protein comprises two null alleles for the mouse IL-3 (the allele comprising the nucleic acid sequence).

Although embodiments employing a human IL-3 gene in a mouse are extensively discussed herein, other non-human animals (e.g., rodents, e.g., rats) that comprise a a human IL-3 gene are also provided.

Human IL-3 polypeptides, loci encoding human IL-3 polypeptides and non-human animals expressing human IL-3 polypeptides are described in WO2011/044050, WO 2014/039782 and WO 2014/071397, each of which is incorporated by reference herein.

### Humanized IL15 Loci

In some aspects, the genetically modified non-human animals provided herein further express a human IL-15 protein encoded by a nucleic acid operably linked to a IL-15 promoter. As used herein, “human IL-15 protein”, means a protein that is a wild-type (or native) human IL-15 protein or a variant of a wild-type (or native) human IL-15 protein, which retains one or more signaling functions of a wild-type (or native) human IL-15 protein, e.g., which allows for stimulation of (or signaling via) the human IL-15 receptor, and/or which is capable of binding to the human IL-15 receptor alpha subunit of the human IL-15 receptor, and/or which is capable of binding to IL-2R beta/IL-15R beta and the common γ-chain (γc). Also encompassed by the term “human IL-15 protein” are fragments of a wild-type human IL-15 protein (or variants thereof), which retain one or more signaling functions of a wild-type human IL-15 protein, e.g., a fragment of a human IL-15 protein, which allows for stimulation of (or signaling via) the human IL-15 receptor, and/or which is capable of binding to the human IL-15 receptor alpha subunit of the human IL-15 receptor, and/or which is capable of binding to IL-2R beta/IL-15R beta and the common γ-chain (γc).

The term “human IL-15 protein” also encompasses fusion proteins, i.e., chimeric proteins, which include one or more fragments of a wild-type human IL-15 protein (or a variant thereof) and which retain one or more signaling functions of a wild-type human IL-15 protein, e.g., as described above. A fusion protein which includes one or more fragments of a wild-type human IL-15 protein (or a variant thereof) may also be referred to herein as a humanized IL-15 protein.

A nucleic acid sequence that encodes a human IL-15 protein is, therefore, a polynucleotide that includes a coding sequence for a human IL-15 protein, i.e., a wild-type human IL-15 protein, a variant of a wild-type human IL-15 protein, a fragment of a wild-type human IL-15 protein (or a variant thereof) which retains one or more signaling functions of a wild-type human IL-15 protein, or fusion proteins, i.e., chimeric proteins, which include one or more fragments of a wild-type human IL-15 protein (or a variant thereof) and which retain one or more signaling functions of a wild-type human IL-15 protein, e.g., as described above.

IL-15 (also known as “Interleukin 15”) is a cytokine that stimulates the proliferation of T lymphocytes. Polypeptide sequence for wild-type human IL-15 and the nucleic acid sequence that encodes wild-type human IL-15 may be found at Genbank Accession Nos. NP_000576.1 and NM_000585.5 (isoform 1 and transcript variant 3), NP 751915.1 and NM_172175.3 (isoform 2 and transcript variant 2). The genomic locus encoding the wild-type human IL-15 protein may be found in the human genome at Chromosome 4; NC 000004.12 (141636583-141733987) or NG_029605.2 (4988-102392). In some embodiments, the human IL-15 locus (e.g., NM_000585.5) includes 8 exons, with exons 3-8 being coding exons. As such, in some embodiments, a nucleic acid sequence including coding sequence for human IL-15 includes one or more of exons 3-8 of the human IL-15 gene. In some instances, the nucleic acid sequence also includes aspects of the genomic locus of the human IL-15, e.g., introns, 3′ and/or 5′ untranslated sequence (UTRs). In some instances, the nucleic acid sequence includes whole regions of the human IL-15 genomic locus. In some instances, the nucleic acid sequence includes exons 5-8 of the human IL-15 genomic locus (i.e., coding exons 3-6).

In the humanized IL-15 non-human animals described herein, the nucleic acid sequence that encodes a human IL-15 protein is operably linked to one or more regulatory sequences of an IL-15 gene, e.g., a regulatory sequence of an IL-15 gene of the non-human animal. Non-human animal, e.g., mouse, IL-15 regulatory sequences are those sequences of the non-human animal IL-15 genomic locus that regulate the non-human animal IL-15 expression, for example, 5′ regulatory sequences, e.g., the IL-15 promoter, IL-15 5′ untranslated region (UTR), etc.; 3′ regulatory sequences, e.g., the 3 'UTR; and enhancers, etc. Mouse IL-15 is located on Chromosome 8, NC_000074.7 (c83129883-83058253), and the mouse IL-15 coding sequence may be found at Genbank Accession Nos. NM_008357.3 (transcript variant 1); NM_001254747.2 (transcript variant 2). The regulatory sequences of mouse IL-15 are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human IL-15 protein is located at the mouse IL-15 genomic locus, the regulatory sequences operably linked to the human IL-15 coding sequence are endogenous, or native, to the mouse genome, i.e., they were present in the mouse genome prior to integration of human nucleic acid sequences.

In some instances, the humanized IL-15 non-human animal, e.g., mouse, is generated by the random integration, or insertion, of a human nucleic acid sequence encoding a human IL-15 protein (including fragments as described above), i.e., a “human IL-15 nucleic acid sequence”, or “human IL-15 sequence”, into the genome of the non-human animal. Typically, in such embodiments, the location of the nucleic acid sequence encoding a human IL-15 protein in the genome is unknown. In other instances, the humanized IL-15 non-human animal is generated by the targeted integration, or insertion, of human IL-15 nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a humanized IL-15 mouse including a nucleic acid sequence that encodes a human IL-15 protein created by targeting human IL-15 nucleic acid sequence to the mouse IL-15 locus, human IL-15 nucleic acid sequence may replace some or all of the mouse sequence, e.g., exons and/or introns, at the IL-15 locus. In some such instances, a human IL-15 nucleic acid sequence is integrated into the mouse IL-15 locus such that expression of the human IL-15 sequence is regulated by the native, or endogenous, regulatory sequences at the mouse IL-15 locus. In other words, the regulatory sequence(s) to which the nucleic acid sequence encoding a human IL-15 protein is operably linked are the native IL-15 regulatory sequences at the mouse IL-15 locus.

In some instances, the integration of a human IL-15 sequence does not affect the transcription of the gene into which the human IL-15 sequence has integrated. For example, if the human IL-15 sequence integrates into a coding sequence as an intein, or the human IL-15 sequence includes a 2A peptide, the human IL-15 sequence will be transcribed and translated simultaneously with the gene into which the human IL-15 sequence has integrated. In other instances, the integration of the human IL-15 sequence interrupts the transcription of the gene into which the human IL-15 sequence has integrated. For example, upon integration of the human IL-15 sequence by homologous recombination, some or all of the coding sequence at the integration locus may be removed, such that the human IL-15 sequence is transcribed instead. In some such instances, the integration of a human IL-15 sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele is indistinguishable from a deletion of the entire locus.

In some instances, the humanized IL-15 non-human animal, e.g., mouse, includes one copy of the nucleic acid sequence encoding a human IL-15 protein. For example, the non-human animal may be heterozygous for the nucleic acid sequence. In other words, one allele at a locus will include the nucleic acid sequence, while the other will be the endogenous allele. For example, as discussed above, in some instances, a human IL-15 nucleic acid sequence is integrated into the non-human animal, e.g., mouse, IL-15 locus such that it creates a null allele for the non-human animal IL-15. In some such embodiments, the humanized IL-15 non-human animal may be heterozygous for the nucleic acid sequence encoding human IL-15, i.e., the humanized IL-15 non-human animal includes one null allele for the non-human animal IL-15 (the allele including the nucleic acid sequence) and one endogenous IL-15 allele (wild-type or otherwise). In other instances, the humanized IL-15 includes two copies of the nucleic acid sequence encoding a human IL-15 protein. For example, the non-human animal, e.g., mouse, may be homozygous for the nucleic acid sequence, i.e., both alleles for a locus in the diploid genome will include the nucleic acid sequence, i.e., the humanized IL-15 non-human animal includes two null alleles for the non-human animal IL-15 (the allele including the nucleic acid sequence).

Although embodiments employing a human IL-15 gene in a mouse are extensively discussed herein, other non-human animals (e.g., rodents, e.g., rats) that comprise a a human IL-15 gene are also provided.

Human IL-15 polypeptides, loci encoding human IL-15 polypeptides and non-human animals expressing human IL-15 polypeptides are described in WO 2016/168212, which is incorporated by reference herein.

### Humanized Liver

In some aspects, the genetically modified non-human animals provided herein further comprises modifications to the animal's immune system such that it is unable or has reduced ability to mount an immune response to xenografted cells (e.g., human hepatocytes) (e.g., the non-human animal is immunodeficient). In some cases, the animals are suitable for xenotransplantation of hepatocytes. In some cases, the animals are genetically modified non-human animals in which (1) non-human (i.e., endogenous) hepatocytes in the liver can be selectively and conditionally ablated; and (2) the non-human animal's immune system has been modified such that it is unable to mount an immune response to xenografted cells (e.g., human hepatocytes) (e.g., the non-human animal is immunodeficient). Also provided are cells and genomes comprising the genetic modifications disclosed herein. The genetically modified non-human animals disclosed herein can be used for in vivo engraftment and expansion of xenotransplanted hepatocytes (e.g., human hepatocytes). Xenotransplantation refers to the transplantation of living cells, tissues, or organs from one species to another. Such cells, tissues, or organs are called xenografts or xenotransplants (e.g., xenotransplanted cells).

Any suitable immunodeficient non-human animal can be used. See, e.g., Weber et al. (2009) Liver Transplantation 15:7-14, herein incorporated by reference in its entirety for all purposes. Such non-human animals can be immunocompromised such that native non-human T cells and B cells do not develop. For examples, such immunodeficient non-human animals can lack functional native non-human T cells, B cells, and/or natural killer (NK) cells. Immunodeficient non-human animals refer to non-human animals lacking in at least one essential function of the immune system. For example, an immunodeficient non-human animal is one lacking specific components of the immune system or lacking function of specific components of the immune system (such as, for example, native non-human B cells, T cells, or NK cells). In some cases, an immunodeficient animal lacks native non-human macrophages. In some cases, an immunodeficient animal comprises one or more genetic alterations that prevent or inhibit the development of functional immune cells (such as native non-human B cells, T cells, or NK cells). Various immunodeficient animal models are known in the art, such as animals in genetic alterations in Rag genes (e.g., the immunodeficient non-human animal is Rag1^{-/-} and/or Rag2^{-/-}), and Il2rg gene (Il2rg^{-/-}) (See, Traggiai et al. (2004) Science, 304:104, incorporated herein by reference); animals with severe combined immunodeficiency (SCID) mutation in the Prkdc gene (Prkdc^{scid} or SCID) (see, e.g., Mercer et al. (2001) Nat. Med. 7(8):927-933, herein incorporated by reference in its entirety for all purposes); animals with SCID mutation and an inactivated endogenous Il2rg gene, such as the NOG background (see, e.g., Ito et al. (2002) Blood 100(9):3175-3182, herein incorporated by reference in its entirety for all purposes); or animals with other mutations such as X-linked SCID characterized by autosomal recessive SCID characterized by JAK3 mutations, ADA mutations, IL7R mutations, CD3 mutations, ARTEMIS (DCLRE1C) mutations, and CD45 (PTPRC) mutations. Although certain non-limiting examples of genetic alterations that result in immunodeficiency are provided above, other known immunodeficient non-human animals can also be used.

Any suitable genetic modification to allow for non-human (i.e., endogenous) hepatocytes in the liver to be selectively and conditionally ablated can be used. In some cases, the non-human animals comprise an inactivated endogenous Fah gene. This inactivation results in a toxic accumulation of tyrosine catabolites within hepatocytes. The compound 2-(2-nitro-4-trifluoro-methylbenzoyl)-1,3-cyclohexanedione (NTBC) can be used to block the enzyme hydroxyphenylpyruvate dioxygenase upstream of FAH and therefore prevents the accumulation of hepatotoxic metabolites. In some cases, the non-human animals comprise a urokinase type plasminogen activator gene (Platt; NCBI GeneID 18792 in mice) operably linked to a liver-specific promoter, such as an albumin promoter. See, e.g., Mercer et al. (2001) Nat. Med. 7(8):927-933, herein incorporated by reference in its entirety for all purposes. Such non-human animals have hepatotoxicity leading to liver failure. In some cases, the non-human animals comprise a herpes simplex virus type 1 thymidine kinase (HSVtk) gene operably linked to a liver-specific promoter, such as an albumin promoter. See, e.g., Hasegawa et al. (2011) Biochem. Biophys. Res. Commun. 405(3):405-410, herein incorporated by reference in its entirety for all purposes. Administration of ganciclovir (GCV), a drug that is not toxic to human or mouse tissues, induces tissue-specific ablation of transgenic liver parenchymal cells. Because HSVtk catalyzes GCV phosphorylation, which is the rate-limiting step that cannot be performed in mammalian cells lacking this transgene, liver cells expressing the transgene are selectively destroyed. Although certain non-limiting examples of genetic modifications to allow for non-human (i.e., endogenous) hepatocytes in the liver to be selectively and conditionally ablated are provided above, any other suitable genetic modifications can also be used.

In one example, genetically modified non-human animals for xenotransplantation of hepatocytes (e.g., transplantation of human hepatocytes) have the following genes inactivated (i.e., knocked out): Fah (encodes fumarylacetoacetase); Rag1 (encodes V(D)J recombination-activating protein 1) and/or Rag2 (encodes V(D)J recombination-activating protein 2); and Il2rg (encodes interleukin 2 receptor subunit gamma). Also provided are genetically modified non-human animal cells or genomes having the following genes inactivated (i.e., knocked out): Fah; Rag1 and/or Rag2; and Il2rg. In one example, the genetically modified non-human animal is a rat. See, e.g., Carbonaro et al. (2022) Sci. Rep. 12(1):14079 and US 2016/0249591, each of which is herein incorporated by reference in its entirety for all purposes. In another example, the genetically modified non-human animal is a mouse. See, e.g., Strom et al. (2010) Methods Mol. Biol. 640:491-509, Azuma et al. (2007) Nat. Biotechnol. 25(8):903-910, and U.S. Pat. No. 8,569,573, each of which is herein incorporated by reference in its entirety for all purposes. In another example, the genetically modified non-human animal is a pig. See, e.g., U.S. Pat. No. 9,000,257, herein incorporated by reference in its entirety for all purposes.

Fah is an essential gene in the tyrosine catabolism pathway. When Fah is mutated in animals, toxic intermediate metabolites accumulate in the liver, causing hepatocyte loss and, ultimately, liver failure and death. This toxicity can be ameliorated by blocking the activity of another tyrosine catabolism enzyme, 4-hydroxyphenylpyruvate dioxygenase, which can be achieved by the administration of the small molecule nitisinone (NTBC). Fah mutant mice are healthy and viable when NTBC is administered. They quickly become moribund and die, however, when NTBC is withdrawn. Thus, NTBC administration and withdrawal allows precise temporal control of hepatocyte toxicity in Fah mutant non-human animals.

Fumarylacetoacetase (also known as FAH, FAA, beta-diketonase, or fumarylacetoacetate hydrolase) is encoded by the Fah gene (also known as fumarylacetoacetate hydrolase). FAH is an enzyme required in the last step of the tyrosine catabolic pathway, which hydrolyzes fumarylacetoacetate into fumarate and acetoacetate. A deficiency in Fah leads to the accumulation of toxic metabolites, including fumarylacetoacetate and maleylacetoacetate. 2-(2-Nitro-4-trifluoromethylbenzoyl)-1,3-cyclohexanedione (nitisinone or NTBC) as an inhibitor of 4-hydroxyphenylpyruvate dioxygenase acts by blocking the accumulation of toxic metabolites such as fumarylacetoacetate and maleylacetoacetate and can be effective in ameliorating liver and kidney damage in human patients with Fah deficiency. Several Fah mutations have been found that cause tyrosinemia type I (type 1 hereditary tyrosinemia, or HT) in humans. This condition is characterized by severe liver and kidney disease, neurological problems, and other signs and symptoms that begin in infancy. The altered Fah gene that causes this condition produces an unstable or inactive enzyme, which results in reduced or absent fumarylacetoacetate hydrolase activity. Without sufficient fumarylacetoacetate hydrolase activity, tyrosine and its byproducts are not properly broken down. As a result, fumarylacetoacetate accumulates in the liver and kidneys. Elevated levels of fumarylacetoacetate are thought to be toxic to cells and accumulation of this substance likely causes the liver and kidney problems and other features that are characteristic of tyrosinemia type I.

Mouse Fah maps to 7 D3; 7 48.36 cM on chromosome 7 (NCBI RefSeq Gene ID 14085; Assembly GRCm39 (GCF_000001635.27); location NC_000073.7 (84234367 . . . 84255150, complement). Reference to the mouse Fah gene includes the canonical, wild type form as well as all allelic forms and isoforms. The canonical, wild type mouse FAH protein has been assigned UniProt accession number P35505 and NCBI Accession No. NP_034306.2. Reference to mouse FAH proteins includes canonical, wild type forms as well as all allelic forms and isoforms. An mRNA (cDNA) encoding the canonical isoform is assigned NCBI Accession No. NM_010176.4. Reference to the mouse Fah mRNA (cDNA) and coding sequence includes the canonical, wild type forms as well as all allelic forms and isoforms.

Rat Fah maps to 1q31 on chromosome 1 (NCBI RefSeq Gene ID 29383; Assembly mRatBN7.2 (GCF_015227675.2); location NC_051336.1 (138548830 . . . 138571599, complement). Reference to the rat Fah gene includes the canonical, wild type form as well as all allelic forms and isoforms. The canonical, wild type rat FAH protein has been assigned UniProt accession number P25093 and NCBI Accession No. NP_058877.1. Reference to rat FAH proteins includes canonical, wild type forms as well as all allelic forms and isoforms. An mRNA (cDNA) encoding the canonical isoform is assigned NCBI Accession No. NM_017181.2. Reference to the rat Fah mRNA (cDNA) and coding sequence includes the canonical, wild type forms as well as all allelic forms and isoforms.

An inactivated endogenous Fah gene is a Fah gene that does not produce a FAH protein or does not produce a functional FAH protein. The non-human animal (or cell or genome) can comprise the inactivated Fah gene in its germline. The non-human animal (or cell or genome) can be homozygous for an inactivating mutation in the Fah gene. As one example, an inactivated endogenous Fah gene can comprise an insertion, a deletion, or one or more point mutations in the endogenous Fah gene resulting in loss of expression of functional FAH protein. Some inactivated endogenous Fah genes can comprise a deletion or disruption of all of the endogenous Fah gene or can comprise a deletion or disruption of a fragment of (i.e., a part of or portion of) the endogenous Fah gene. For example, some, most, or all of the coding sequence in the endogenous Fah gene can be deleted or disrupted. In one example, a 5′ fragment of the Fah gene can be deleted or disrupted (e.g., including the start codon). As one example, an inactivated endogenous Fah gene can be one in which the start codon of the endogenous Fah gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Fah gene can be deleted or disrupted (e.g., including the stop codon). In another example, an internal fragment of the Fah gene (i.e., a fragment from the middle of the Fah gene) can be deleted or disrupted. In another example, all of the coding sequence in the endogenous Fah gene is deleted or disrupted.

Inactivated endogenous Fah gene non-human animals, and non-human animals deficient in expressing Fah are described in US2016/024959, U.S. Pat. No. 8,569,573, Azuma et al. Nature Biotechnology 25(8):903-10 (2007), Carbonaro et al. Scientific Reports 12:14079-89 (2022), Carbonaro et al. Sci. Adv. 9, eadf4490 (2023), and Wilson et al. Stem Cell Research 13:404-412 (2014), which are incorporated by reference herein.

Genetically modified non-human animals comprising various modifications described herein such as Rag 2 (and optionally Rag1) knock out, Il2rg KO, Hmox-1 KO, Fah KO, and humanizations of genes including Sirpa and others (such as e.g., M-CSF, CD47, etc. described here) may be bred together, engrafted with human HSCs and human hepatocytes, and used as a model for studying human parasitic infections with both liver and blood stages, e.g., malaria.

### Genetically Modified Non-Human Animals and ES Cells

In certain aspects, provided herein are genetically modified non-human animals (e.g., rodents, such as rats or mice) comprising: (i) a Rag1 and/or Rag2 gene knock-out; (ii) a IL2rg gene knock-out; and (iii) a homozygous null mutation in the non-human animal Heme oxygenase-1 (Hmox-1) gene, and optionally a Fah gene knock-out and/or one or more of the humanized loci disclosed herein as well as genetically modified non-human animal ES cells useful in the making of such non-human animals.

In certain aspects, provided herein are genetically modified non-human animals (e.g., rodents, such as rats or mice) and non-human animal (e.g., rodent, such as rat or mouse) ES cells comprising in their germline and/or genome: (i) a Rag1 and/or Rag2 gene knock-out; (ii) a IL2rg gene knock-out; and (iii) a homozygous null mutation in the non-human animal Heme oxygenase-1 (Hmox-1) gene, and optionally one or more of the engineered loci described herein. In some embodiments, provided herein are genetically modified non-human animals (e.g., mice) and non-human animal (e.g., mouse) ES cells comprising in their germline and/or genome: (i) a Rag2 gene knock-out; (ii) a IL2rg gene knock-out; and (iii) a homozygous null mutation in the non-human animal Heme oxygenase-1 (Hmox-1) gene, and optionally one or more of the engineered loci described herein. In some embodiments, provided herein are genetically modified non-human animals (e.g., rats) and non-human animal (e.g., rat) ES cells comprising in their germline and/or genome: (i) a Rag1 gene knock-out; (ii) a Rag2 gene knock-out; (iii) a IL2rg gene knock-out; and (iv) a homozygous null mutation in the non-human animal Heme oxygenase-1 (Hmox-1) gene, and optionally one or more of the engineered loci described herein. For example, in some embodiments the non-human animal or ES cell comprises in its germline and/or genome a humanized Sirpa locus provided herein. In some embodiments, the non-human animal or ES cell comprises in their germline and/or genome a CD47 locus provided herein. In certain embodiments, the non-human animal or ES cell comprises in their germline and/or genome an M-CSF locus provided herein. In certain embodiments, the non-human animal or ES cell comprises in their germline and/or genome a GM-CSF locus provided herein. In certain embodiments, the non-human animal or ES cell comprises in their germline and/or genome a TPO locus provided herein. In certain embodiments, the non-human animal or ES cell comprises in their germline and/or genome an EPO locus provided herein. In certain embodiments, the non-human animal or ES cell comprises in their germline and/or genome an IL-3 locus provided herein. In certain embodiments, the non-human animal or ES cell comprises in their germline and/or genome an IL-15 locus provided herein. In certain embodiments, the non-human animal or ES cell comprises in their germline and/or genome an inactivation (e.g., a deletion) of Fah gene provided herein. In some embodiments, the non-human animal or ES cell is heterozygous for one or more of the loci, e.g., genetically engineered loci, provided herein. In some embodiments, the non-human animal or ES cell is homozygous for one or more of the loci, e.g., genetically engineered loci, provided herein.

In some embodiments, the non-human animal can be any non-human animal. In some embodiments, the non-human animal is a vertebrate. In some embodiments, the non-human animal is a mammal. In some embodiments, the genetically modified non-human animal described herein may be selected from a group consisting of a mouse, rat, rabbit, pig, bovine (e.g., cow, bull, buffalo), deer, sheep, goat, llama, chicken, cat, dog, ferret, primate (e.g., marmoset, rhesus monkey). For non-human animals where suitable genetically modifiable ES cells are not readily available, other methods can be employed to make a non-human animal comprising the genetic modifications described herein. Such methods include, for example, modifying a non-ES cell genome (e.g., a fibroblast or an induced pluripotent cell) and employing nuclear transfer to transfer the modified genome to a suitable cell, such as an oocyte, and gestating the modified cell (e.g., the modified oocyte) in a non-human animal under suitable conditions to form an embryo.

In some embodiments, the non-human animal is a mammal. In some embodiments, the non-human animal is a small mammal, e.g., of the superfamily Dipodoidea or Muroidea. In some embodiments, the non-human animal is a rodent. In certain embodiments, the rodent is a mouse, a rat or a hamster. In some embodiments, the rodent is selected from the superfamily Muroidea. In some embodiments, the non-human animal is from a family selected from Calomyscidae (e.g., mouse-like hamsters), Cricetidae (e.g., hamster, New World rats and mice, voles), Muridae (e.g., true mice and rats, gerbils, spiny mice, crested rats), Nesomyidae (e.g., climbing mice, rock mice, white-tailed rats, Malagasy rats and mice), Platacanthomyidae (e.g., spiny dormice), and Spalacidae (e.g., mole rates, bamboo rats, and zokors). In some embodiments, the rodent is selected from a true mouse or rat (family Muridae), a gerbil, a spiny mouse, and a crested rat. In some embodiments, the mouse is from a member of the family Muridae. In some embodiments, the non-human animal is a rodent. In some embodiments, the rodent is selected from a mouse and a rat. In some embodiments, the non-human animal is a mouse.

In some embodiments, the non-human animal is a mouse of a C57BL strain. In some embodiments, the C57BL strain is selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the non-human animal is a mouse of a 129 strain. In some embodiments, the 129 strain is selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. In some embodiments, the genetically modified mouse is a mix of a 129 strain and a C57BL strain. In some embodiments, the mouse is a mix of 129 strains and/or a mix of C57BL/6 strains. In some embodiments, the 129 strain of the mix is a 129S6 (129/SvEvTac) strain. In some embodiments, the mouse is a BALB strain (e.g., BALB/c). In some embodiments, the mouse is a mix of a BALB strain and another strain (e.g., a C57BL strain and/or a 129 strain). In some embodiments, the non-human animals provided herein can be a mouse derived from any combination of the aforementioned strains.

In some embodiments, the non-human animal provided herein is a rat. In some embodiments, the rat is selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

In certain embodiments, the genetically modified non-human animals or ES cells comprise in their genome and/or germline multiple loci provided herein, such as multiple genetically engineered loci provided herein. For example, in some embodiments the non-human animal or ES cell comprises in its germline and/or genome: (i) a Rag1 and/or Rag2 gene knock-out; (ii) a IL2rg gene knock-out; and (iii) a homozygous null mutation in the non-human animal Heme oxygenase-1 (Hmox-1) gene. In some embodiments the non-human animal or ES cell comprises in its germline and/or genome (i) a Rag1 and/or Rag2 gene knock-out; (ii) a IL2rg gene knock-out; and (iii) a homozygous null mutation in the non-human animal Heme oxygenase-1 (Hmox-1) gene, and a humanized Sirpa locus provided herein. In some embodiments, the non-human animal or ES cell comprises in its germline and/or genome (i) a Rag1 and/or Rag2 gene knock-out; (ii) a IL2rg gene knock-out; and (iii) a homozygous null mutation in the non-human animal Heme oxygenase-1 (Hmox-1) gene, a humanized Sirpa locus provided herein, and a humanized M-CSF locus provided herein. In some embodiments, the non-human animal or ES cell comprises in its germline and/or genome (i) a Rag1 and/or Rag2 gene knock-out; (ii) a IL2rg gene knock-out; and (iii) a homozygous null mutation in the non-human animal Heme oxygenase-1 (Hmox-1) gene, a humanized Sirpa locus provided herein, a humanized M-CSF locus provided herein, and a humanized CD47 locus provided herein. In some embodiments the non-human animal or ES cell comprises in its germline and/or genome (i) a Rag1 and/or Rag2 gene knock-out; (ii) a IL2rg gene knock-out; and (iii) a homozygous null mutation in the non-human animal Heme oxygenase-1 (Hmox-1) gene, a humanized Sirpa locus provided herein, and optionally one or more humanized loci selected from the group consisting of a humanized CD47 locus provided herein, a humanized M-CSF locus provided herein, a humanized GM-CSF locus provided herein, a humanized TPO locus provided herein, a humanized EPO locus provided herein, a humanized IL-3 locus provided herein, a humanized IL-15 locus provided herein, and any combinations thereof. In some embodiments, the non-human animal or ES cell described herein further comprises in its germline and/or genome an inactivation (e.g., a deletion) of the Fah gene.

In certain aspects, the genetically modified non-human animal does not express Hmox-1 polypeptide. In certain aspects, the genetically modified non-human animal expresses one or more of the human or humanized polypeptides encoded by the humanized loci provided herein. For example, in some embodiments the non-human animal expresses a human or humanized Sirpa polypeptide. In certain embodiments, the non-human animal expresses a human or humanized CD47 polypeptide. In certain embodiments, the non-human animal expresses a human or humanized M-CSF polypeptide. In certain embodiments, the non-human animal expresses a human or humanized GM-CSF polypeptide. In certain embodiments, the non-human animal expresses a human or humanized TPO polypeptide. In certain embodiments, the non-human animal expresses a human or humanized EPO polypeptide. In certain embodiments, the non-human animal or ES cell the non-human animal expresses a human or humanized IL-3 polypeptide. In certain embodiments, the non-human animal expresses a human or humanized IL-15 polypeptide. In certain embodiments, the non-human animal or ES cell does not express an FAH protein. In certain embodiments, the genetically modified non-human animal does not express Hmox-1 protein and FAH protein.

The genetically modified non-human animals and ES cells can be generated using any appropriate method known in the art. For example, such genetically modified non-human animal ES cells can be generated using VELOCIGENE® technology, which is described in U.S. Pat. Nos. 6,586,251, 6,596,541, 7,105,348, and Valenzuela et al. (2003) “High-throughput engineering of the mouse genome coupled with high-resolution expression analysis” Nat. Biotech. 21(6): 652-659, each of which is hereby incorporated by reference. Modifications can also be made using a genome targeted nuclease system, such as a CRISPR/Cas system, a transcription activator-like effector nuclease (TALEN) system or a zinc finger nuclease (ZFN) system. In some embodiments, modifications are made using a CRISPR/Cas system, as described, for example, in U.S. patent application Ser. Nos. 14/314,866, 14/515,503, 14/747,461 and 14/731,914, each of which is incorporated by reference. Genetically modified rat ES cells and rats can be made according to US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties). Exemplary methods of making such genetically modified non-human animals and ES cells are also provided herein in Example 1.

ES cells described herein can then be used to generate a non-human animal using methods known in the art. For example, the mouse non-human animal ES cells described herein can be used to generate genetically modified mice using the VELOCIMOUSE® method, as described in U.S. Pat. No. 7,294,754 and Poueymirou et al., Nature Biotech 25:91-99 (2007), each of which is hereby incorporated by reference. Resulting mice can be bread to homozygosity.

### Methods of Making Genetically Modified Non-Human Animals and ES Cells

In certain aspects, provided herein are methods of making non-human animals (e.g., a mouse or a rat) and ES cells that comprise one or more of the genetically modified loci provided here. For example, in some embodiments provided herein are methods of making non-human animals (e.g., a mouse or a rat) and ES cells that comprise (i) a Rag1 and/or Rag2 gene knock-out; (ii) a IL2rg gene knock-out; and (iii) a homozygous null mutation in the non-human animal Heme oxygenase-1 (Hmox-1) gene. In some embodiments, provided herein are methods of making non-human animals (e.g., a mouse or a rat) and ES cells that comprise (i) a Rag1 and/or Rag2 gene knock-out; (ii) a IL2rg gene knock-out; and (iii) a homozygous null mutation in the non-human animal Heme oxygenase-1 (Hmox-1) gene, and further comprise a humanized Sirpa locus provided herein. In some embodiments, provided herein are methods of making non-human animals (e.g., a mouse or a rat) and ES cells that further comprise a humanized CD47 locus provided herein, a humanized M-CSF locus provided herein, a humanized GM-CSF locus provided herein, a humanized TPO locus provided herein, a humanized EPO locus provided herein, a humanized IL-3 locus provided herein, and/or a humanized IL-15 locus provided herein. In some embodiments provided herein are methods of making non-human animals (e.g., a mouse or a rat) and ES cells that comprise a homozygous null mutation in the Fah gene. The exemplary methods of making genetically modified non-human animals and ES cells provided herein are described in the description, examples, and/or figures herein. The generation of a non-human animal comprising a null mutation in the non-human animal Hmox-1 gene may be accomplished using any convenient method for the making genetically modified animals, e.g., as known in the art or as described herein in Example 1.

The generation of a non-human animal comprising a nucleic acid sequence that encodes a human or humanized protein (e.g., hSIRPA, hCD47, hM-CSF, hGM-CSF, hTPO, hEPO, hIL-3, or hIL-15) may be accomplished using any convenient method for the making genetically modified animals, e.g., as known in the art or as described herein.

For example, a nucleic acid encoding the human or humanized protein (e.g., hSIRPA, hCD47, hM-CSF, hGM-CSF, hTPO, hEPO, hIL-3, and/or hIL-15) may be incorporated into a recombinant vector in a form suitable for insertion into the genome of the host cell and expression of the human protein in a non-human host cell. In various embodiments, the recombinant vector may include the one or more regulatory sequences operatively linked to the nucleic acid encoding the human protein in a manner which allows for transcription of the nucleic acid into mRNA and translation of the mRNA into the human protein, as described above. It will be understood that the design of the vector may depend on such factors as the choice of the host cell to be transfected and/or the amount of human protein to be expressed.

Any of various methods may then be used to introduce the human nucleic acid sequence into an animal cell to produce a genetically modified animal that expresses the human gene. Such techniques are well-known in the art and include, but are not limited to, pronuclear microinjection, transformation of embryonic stem cells, homologous recombination and knock-in techniques. Methods for generating genetically modified animals that can be used include, but are not limited to, those described in Sundberg and Ichiki (2006, Genetically Engineered Mice Handbook, CRC Press), Hofker and van Deursen (2002, Genetically modified Mouse Methods and Protocols, Humana Press), Joyner (2000, Gene Targeting: A Practical Approach, Oxford University Press), Turksen (2002, Embryonic stem cells: Methods and Protocols in Methods Mol Biol, Humana Press), Meyer et al. (2010, Proc. Nat. Acad. Sci. USA 107: 15022-15026), and Gibson (2004, A Primer Of Genome Science 2nd ed. Sunderland, Massachusetts: Sinauer), U.S. Pat. No. 6,586,251, Rathinam et al. (2011, Blood 118:3119-28), Willinger et al, (2011, Proc Natl Acad Sci USA, 108:2390-2395), Rongvaux et al, (2011, Proc Natl Acad Sci USA, 108:2378-83) and Valenzuela et al. (2003, Nat Biot 21:652-659).

For example, the subject genetically modified animals can be created by introducing the nucleic acid encoding the human protein into an oocyte, e.g., by microinjection, and allowing the oocyte to develop in a female foster animal. In preferred embodiments, the expression is injected into fertilized oocytes. Fertilized oocytes can be collected from superovulated females the day after mating and injected with the expression construct. The injected oocytes are either cultured overnight or transferred directly into oviducts of 0.5-day p.c. pseudopregnant females. Methods for superovulation, harvesting of oocytes, expression construct injection and embryo transfer are known in the art and described in Manipulating the Mouse Embryo (2002, A Laboratory Manual, 3rd edition, Cold Spring Harbor Laboratory Press). Offspring can be evaluated for the presence of the introduced nucleic acid by DNA analysis (e.g., PCR, Southern blot, DNA sequencing, etc.) or by protein analysis (e.g., ELISA, Western blot, etc.).

As another example, the construct comprising the nucleic acid sequence encoding the human protein may be transfected into stem cells (e.g., ES cells or iPS cells) using well-known methods, such as electroporation, calcium-phosphate precipitation, lipofection, etc. The cells can be evaluated for the presence of the introduced nucleic acid by DNA analysis (e.g., PCR, Southern blot, DNA sequencing, etc.) or by protein analysis (e.g., ELISA, Western blot, etc.). Cells determined to have incorporated the expression construct can then be introduced into preimplantation embryos. For a detailed description of methods known in the art useful for the compositions and methods of the invention, see Nagy et al, (2002, Manipulating the Mouse Embryo: A Laboratory Manual, 3rd edition, Cold Spring Harbor Laboratory Press), Nagy et al. (1990, Development 110:815-821), U.S. Pat. Nos. 7,576,259, 7,659,442, 7,294,754, and Kraus et al. (2010, Genesis 48:394-399).

Additionally, as described in some of the Examples below, a nucleic acid construct may be constructed using VELOCIGENE® genetic engineering technology (see, e.g., Valenzuela et al. (2003) High throughput engineering of the mouse genome coupled with high-resolution expression analysis, Nature Biotech. 21(6): 652-59 and U.S. Pat. No. 6,586,251), introduced into stem cells (e.g., ES cells), and correctly targeted clones determined using loss-of-allele and gain-of-allele assays (Valenzuela et al, supra); correctly targeted ES cells may be used as donor ES cells for introduction into an 8-cell stage mouse embryo using the VELOCIMOUSE® method (see, e.g., U.S. Pat. No. 7,294,754 and Poueymirou et al. 2007, F0 generation mice that are essentially fully derived from the donor gene-targeted ES cells allowing immediate phenotypic analyses Nature Biotech. 25(1):91-99). In addition, genetically modified rat ES cells and rats can be made according to US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties).

In some embodiments, genetically modified founder animals can be bred to additional animals carrying one or more genetic modifications. For example, HMOX-1-deficient non-human animals provided herein can further be bred to other genetically modified non-human animals carrying other genetic modifications, which include introduction of either completely human or humanized genes, e.g., hSirpa knock-in mice, hM-CSF knock-in mice, hCD47 knock-in mice, hIL-3 knock-in mice, hGM-CSF knock-in mice, hTPO knock-in mice, hEPO knock-in mice, hIL-15 knock-in mice, and the like, or be bred to knockout animals, e.g., a non-human animal that is deficient for one or more proteins, e.g., does not express one or more of its genes, e.g., a Rag1-deficient animal, a Rag2-deficient animal, an Il2rg-deficient animal, or an FAH-deficient animal.

In another embodiment, stem cells, e.g., ES cells, may be generated such that they comprise several genetic modifications, e.g., humanizations or gene deletions described herein, and such stem cells may be introduced into an embryo to generate genetically modified animals with several genetic modifications.

As discussed above, in some embodiments, the genetically modified non-human animal is an immunodeficient animal. Genetically modified non-human animals that are immunodeficient and comprise one or more human or humanized proteins, e.g., hSIRPA, hIL-3, hGM-CSF, hM-CSF, hEPO, hCD47, hIL-15, and/or hTPO, may be generated using any convenient method for the generation of genetically modified animals, e.g., as known in the art or as described herein. For example, the generation of the genetically modified immunodeficient animal can be achieved by introduction of the nucleic acid encoding the human protein into an oocyte or stem cells comprising a mutant SCID gene allele or Rag and/or Rag2 and Il2rg null alleles that, when homozygous, will result in immunodeficiency as described in greater detail above and in the working examples herein. Mice are then generated with the modified oocyte or ES cells using, e.g., methods described herein and known in the art, and mated to produce the immunodeficient mice comprising the desired genetic modification. As another example, genetically modified non-human animals can be generated in an immunocompetent background, and crossed to an animal comprising a mutant gene allele that, when hemizygous or homozygous, will result in immunodeficiency, and the progeny mated to create an immunodeficient animal expressing the at least one human protein of interest.

In some embodiments, the genetically modified mouse is treated so as to eliminate endogenous hematopoietic cells that may exist in the mouse. In one embodiment, the treatment comprises irradiating the genetically modified mouse. In a specific embodiment, newborn genetically modified mouse pups are irradiated sublethally. In a specific embodiment, newborn pups are irradiated 2×200 cGy with a four-hour interval.

Various embodiments of the invention provide genetically modified animals that include a human nucleic acid in substantially all of their cells, as well as genetically modified animals that include a human nucleic acid in some, but not all their cells. In some instances, e.g., targeted recombination, one copy of the human nucleic acid will be integrated into the genome of the genetically modified animals. In other instances, e.g., random integration, multiple copies, adjacent or distant to one another, of the human nucleic acid may be integrated into the genome of the genetically modified animals.

Thus, in some embodiments, the subject genetically modified non-human animal may be an immunodeficient animal comprising a genome that includes a nucleic acid encoding a human polypeptide operably linked to the corresponding non-human animal promoter, wherein the animal expresses the encoded human polypeptide. In other words, the subject genetically modified immunodeficient non-human animal comprises a genome that comprises a nucleic acid encoding at least one human polypeptide, wherein the nucleic acid is operably linked to the corresponding non-human promoter and a polyadenylation signal, and wherein the animal expresses the encoded human polypeptide.

Additional methods of generating genetically modified non-human animals comprising a genome that includes a nucleic acid encoding one or more human proteins, e.g., hSIRPA, hIL-3, hGM-CSF, hM-CSF, hEPO, hCD47, hIL-15, and/or hTPO, are described in U.S. Pat. No. 11,019,810, US Pat. Publ. No. US 2021/0161112, WO 2011/044050, WO 2012/112544, WO 2014/039782, WO 2014/071397, WO 2015/179317, and WO 2016/168212, each of which each of which is incorporated by reference herein.

### Engraftment

In some embodiments, the subject genetically modified non-human animal is also immunodeficient “Immunodeficient” includes deficiencies in one or more aspects of an animal's native, or endogenous, immune system, e.g., the animal is deficient for one or more types of functioning host immune cells, e.g., deficient for non-human B cell number and/or function, non-human T cell number and/or function, non-human NK cell number and/or function, etc.

One method to achieve immunodeficiency in the subject animals is sublethal irradiation. Alternatively or in addition, immunodeficiency may be achieved by any one of a number of gene mutations known in the art, any of which may be bred either alone or in combination into the subject genetically modified non-human animals of the present disclosure or which may be used as the source of stem cells into which the genetic modifications of the subject disclosure may be introduced. Non-limiting examples include X-linked SCID, associated with IL2RG gene mutations and characterized by the lymphocyte phenotype T(−) B(+) NK(−); autosomal recessive SCID associated with Jak3 gene mutations and characterized by the lymphocyte phenotype T(−) B(+) NK(−); ADA gene mutations characterized by the lymphocyte phenotype T(−) B(−) NK(−); IL-7R alpha-chain mutations characterized by the lymphocyte phenotype T(−) B(+) NK(+); CD3 delta or epsilon mutations characterized by the lymphocyte phenotype T(−) B(+) NK(+); RAG1 and RAG2 mutations characterized by the lymphocyte phenotype T(−) B(−) NK(+); Artemis gene mutations characterized by the lymphocyte phenotype T(−) B(−) NK(+), CD45 gene mutations characterized by the lymphocyte phenotype T(−) B(+) NK(+); and Prkdcscld mutations characterized by the lymphocyte phenotype T(−), B(−). As such, in some embodiments, the genetically modified immunodeficient non-human animal has one or more deficiencies selected from an IL2 receptor gamma chain deficiency, a Jak3 deficiency, an ADA deficiency, an IL7R deficiency, a CD3 deficiency, a RAG1 and/or RAG2 deficiency, an Artemis deficiency, a CD45 deficiency, and a Prkdc deficiency. In one embodiment, the immunodeficiency is achieved by gene mutation or deletion in Rag1 and/or Rag2 and Il2rg genes. These and other animal models of immunodeficiency will be known to the ordinarily skilled artisan, any of which may be used to generate immunodeficient animals of the present disclosure.

In some embodiments, genetically modified non-human animals in accordance with the invention find use as recipients of human hematopoietic cells that are capable of developing human immune cells from engrafted human hematopoietic cells. As such, in some aspects of the invention, the subject genetically modified animal is a genetically modified, immunodeficient, non-human animal that is engrafted with human hematopoietic cells.

Any source of human hematopoietic cells, human hematopoietic stem cells (HSCs) and/or hematopoietic stem progenitor cells (HSPC) as known in the art or described herein may be transplanted into the genetically modified immunodeficient non-human animals of the present disclosure. One suitable source of human hematopoietic cells known in the art is human umbilical cord blood cells, in particular CD34-positive (CD34+) cells. Another source of human hematopoietic cells is human fetal liver. Another source is human bone marrow. Also encompassed are induced pluripotent stem cells (iPSC) and induced hematopoietic stem cells (iHSC) produced by the de-differentiation of somatic cells, e.g., by methods known in the art. Methods for the transplantation of human cells into non-human animals are well-described in the art and elsewhere herein, any of which may be employed by the ordinarily skilled artisan to arrive at the subject genetically modified engrafted non-human animals.

Cell populations of particular interest include those that comprise hematopoietic stem or progenitor cells, which will contribute to or reconstitute the hematopoietic system of the genetically modified non-human animals, for example, peripheral blood leukocytes, fetal liver cells, fetal bone, fetal thymus, fetal lymph nodes, vascularized skin, artery segments, and purified hematopoietic stem cells, e.g., mobilized HSCs or cord blood HSCs.

Cells may be from any mammalian species, e.g., murine, rodent, canine, feline, equine, bovine, ovine, primate, human, etc. In one embodiment, the cells are human cells. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present disclosure are maintained for fewer than 10 passages in vitro.

If the cells are primary cells, they may be harvested from an individual by any convenient method. For example, cells, e.g., blood cells, e.g., leukocytes, may be harvested by apheresis, leukocytapheresis, density gradient separation, etc. As another example, cells, e.g., skin, muscle, bone marrow, spleen, liver, pancreas, lung, intestine, stomach tissue, etc. may be harvested by biopsy. An appropriate solution may be used for dispersion or suspension of the harvested cells. Such solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

In some instances, a heterogeneous population of cells will be transplanted into the genetically modified non-human animals. In other instances, a population of cells that is enriched for a particular type of cell, e.g., a progenitor cell, e.g., a hematopoietic progenitor cell, will be engrafted into the genetically modified non-human animals. Enrichment of a cell population of interest may be by any convenient separation technique. For example, the cells of interest may be enriched by culturing methods. In such culturing methods, particular growth factors and nutrients are typically added to a culture that promote the survival and/or proliferation of one cell population over others. Other culture conditions that affect survival and/or proliferation include growth on adherent or non-adherent substrates, culturing for particular lengths of time, etc. Such culture conditions are well known in the art. As another example, cells of interest may be enriched for by separation the cells of interest from the initial population by affinity separation techniques. Techniques for affinity separation may include magnetic separation using magnetic beads coated with an affinity reagent, affinity chromatography, “panning” with an affinity reagent attached to a solid matrix, e.g., plate, cytotoxic agents joined to an affinity reagent or used in conjunction with an affinity reagent, e.g., complement and cytotoxins, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the cells of interest.

For example, using affinity separation techniques, cells that are not the cells of interest for transplantation may be depleted from the population by contacting the population with affinity reagents that specifically recognize and selectively bind markers that are not expressed on the cells of interest. For example, to enrich for a population of hematopoietic progenitor cells, one might deplete cells expressing mature hematopoietic cell markers. Additionally or alternatively, positive selection and separation may be performed using by contacting the population with affinity reagents that specifically recognize and selectively bind markers associated with hematopoietic progenitor cells, e.g., CD34, CD133, etc. By “selectively bind” is meant that the molecule binds preferentially to the target of interest or binds with greater affinity to the target than to other molecules. For example, an antibody will bind to a molecule comprising an epitope for which it is specific and not to unrelated epitopes. In some embodiments, the affinity reagent may be an antibody, i.e., an antibody that is specific for CD34, CD133, etc. In some embodiments, the affinity reagent may be a specific receptor or ligand for CD34, CD133, etc., e.g., a peptide ligand and receptor; effector and receptor molecules, a T-cell receptor specific for CD34, CD133, etc., and the like. In some embodiments, multiple affinity reagents specific for the marker of interest may be used.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g., phycoerythrin and allophycocyanins, fluorescein and Texas red. Frequently each antibody is labeled with a different fluorochrome, to permit independent sorting for each marker.

The initial population of cells are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent are selected for by any convenient affinity separation technique, e.g., as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for a cell type of interest, e.g., hematopoietic cells, are achieved in this manner. The cells will be about 70%, about 75%, about 80%, about 85% about 90% or more of the cell composition, about 95% or more of the enriched cell composition, and will preferably be about 95% or more of the enriched cell composition. In other words, the composition will be a substantially pure composition of cells of interest.

The cells to be transplanted into the genetically modified non-human animals, be they a heterogeneous population of cells or an enriched population of cells, may be transplanted immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells. Additionally or alternatively, the cells may be cultured in vitro under various culture conditions. Culture medium may be liquid or semi-solid, e.g., containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g., penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The cells may be genetically modified prior to transplanting to the genetically modified non-human animals, e.g., to provide a selectable or traceable marker, to induce a genetic defect in the cells (e.g., for disease modeling), to repair of a genetic defect or ectopically express a gene in the cells (e.g., to determine if such modifications will impact the course of a disease), etc. Cells may be genetically modified by transfection or transduction with a suitable vector, homologous recombination, or other appropriate technique, so that they express a gene of interest, or with an antisense mRNA, siRNA or ribozymes to block expression of an undesired gene. Various techniques are known in the art for the introduction of nucleic acids into target cells. To prove that one has genetically modified the cells, various techniques may be employed. The genome of the cells may be restricted and used with or without amplification. The polymerase chain reaction; gel electrophoresis; restriction analysis; Southern, Northern, and Western blots; sequencing; or the like, may all be employed. General methods in molecular and cellular biochemistry for these and other purposes disclosed in this application can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The cells may be transplanted in the genetically modified non-human animals by any convenient method, including, for example, intra-hepatic injection, tail-vein injection, retro-orbital injection, and the like. Typically, about 0.5×10⁵-2×10⁶ pluripotent or progenitor cells are transplanted, e.g., about 1×10⁵-1×10⁶ cells, or about 2×10⁵-5×10⁵ cells. In some instances, the mouse is sublethally irradiated prior to transplanting the human cells. In other words, the mouse is exposed to a sublethal dose of radiation, e.g., as described in the examples section below and as well-known in the art. The engrafted genetically modified non-human animal is then maintained under laboratory animal husbandry conditions for at least 1 week, e.g., 1 week or more, or two weeks or more, sometimes 4 weeks or more, and in some instances 6 weeks or more, to allow sufficient reconstitution of the immune system with the engrafted cells.

In some embodiments, the transplanted human hematopoietic cells give rise in the genetically modified non-human animal to one or more engrafted human cells selected from a human CD34-positive cell, a human hematopoietic stem cell, a human hematopoietic cell, a myeloid progenitor cell, an erythroid progenitor cell, a myeloid cell, a dendritic cell, a monocyte, a neutrophil, a mast cell, an erythrocyte, and a combination thereof. In one embodiment, the human cell is present at 4 months, 5 months, 6 months, 7 months, 8 months, 9 months, 10 months, 11 months, or 12 months after engraftment. In a specific embodiment, the human cells comprise cells of the erythroid lineage.

In some embodiments, the transplanted human hematopoietic cells give rise in the genetically modified non-human animal to an engrafted human hemato-lymphoid system that comprises human hematopoietic stem and progenitor cells, human myeloid progenitor cells, human myeloid cells, human dendritic cells, human monocytes, human granulocytes, human neutrophils, human mast cells, human erythrocytes, human thymocytes, human T cells, human B cells, and human platelets. In one embodiment, the human hemato-lymphoid system is present at 4 months, 5 months, 6 months, 7 months, 8 months, 9 months, 10 months, 11 months, or 12 months after engraftment. In a specific embodiment, the human hemato-lymphoid system comprises cells of the erythroid lineage.

Cells of the erythroid lineage include erythrocytes and cells that give rise to erythrocytes. “Erythrocytes” include mature red blood cells, also referred to red cells or red corpuscles. Cells that give rise to erythrocytes include erythrocyte progenitor cells, i.e., proliferating multipotent cells, and erythrocyte precursors, i.e., proliferating or nonproliferating cells committed to becoming erythrocytes.

Erythrocytes are the major cellular element of the circulating blood and transport oxygen as their principal function. The number of erythrocytes per cubic millimeter of blood is usually maintained between 4.5 and 5.5 million in men and between 4.2 and 4.8 million in women. It varies with age, activity, and environmental conditions. For example, an increase to a level of 8 million/mm can normally occur at over 10,000 feet above sea level. An erythrocyte normally lives for 110 to 120 days, when it is removed from the bloodstream and broken down by the reticuloendothelial system. New erythrocytes are produced at a rate of slightly more than 1% a day; thus a constant level is usually maintained. Acute blood loss, hemolytic anemia, or chronic oxygen deprivation may cause erythrocyte production to increase greatly.

Erythrocytes originate from hematopoietic stem cells in the marrow of the long bones, developing into erythrocytes through successive cell stages that include common myeloid progenitor cells (CD123+, CD34+, c-kit+, Flt3+); megakaryocyte-erythroid progenitor cells (CD34+, CD38+, CD45RA−); proerythroblasts (also called pronormoblasts if normal, or promegaloblasts if abnormal; large CD71+, EpoR+, c-kit+, Terl 19+ progenitors); basophilic erythroblasts (cytoplasm is basophilic, the nucleus is large with clumped chromatin, and the nucleoli have disappeared); polychromatic erythroblasts (also called an intermediate normoblast, in which the nuclear chromatin shows increased clumping and the cytoplasm begins to acquire hemoglobin and takes on an acidophilic tint); orthochromatic normoblasts (the final stage before nuclear loss, in which the nucleus is small and ultimately becomes a blue-black, homogeneous, structureless mass); and reticulocytes (circulating CD235+, CD71+ cells; the cell is characterized by a meshlike pattern of threads and particles at the former site of the nucleus).

Mature erythrocytes appear on a peripheral smear as biconcave, round or ovoid discs about 6-8μιη in diameter. They contain hemoglobin and have a zone of central pallor due to the cell's biconcavity, and may be readily identified by flow cytometry or immunohistochemistry-based methods by the elevated expression of cell surface markers CD235 and CD59 relative to non-erythroid cells.

In some aspects, the genetically modified non-human animal provided herein is engrafted with human hematopoietic cells and comprises an infection by a human pathogen. Of particular interest in these embodiments are human pathogens that target human cells of the erythroid lineage. Non-limiting examples of such pathogens include protozoans of the genera Plasmodium, Babesia, Theileria, and the like. As described in greater detail below, the subject genetically modified non-human animal engrafted with human hematopoietic cells may be infected with human pathogen using any appropriate method known in the art or described herein for infecting animals with the pathogens of interest. Animals so infected will typically show signs of parasitaemia including altered morphology by Giemsa-stained blood smear, and a severe decrease (e.g., 50%) in total erythrocyte concentration and anemia.

In some aspects, the genetically modified non-human animal provided herein is engrafted with human hematopoietic cells comprising a disease-specific mutation, e.g., a mutation in β-globin gene that leads to sickle cell disease.

### Nonlimiting Applications of Genetically Modified Engrafted Mice

The genetically modified non-human animals of the present disclosure find many uses in the art. For example, engrafted genetically modified animals of the present disclosure are useful for studying human erythropoiesis and the function of human erythrocytes. As another example, engrafted genetically modified mice of the present disclosure provide a useful system for screening candidate agents for desired activities in vivo, for example, to identify agents that are able to modulate (i.e., promote or suppress) human erythropoiesis and/or the function of human erythrocytes, e.g., in a healthy or a diseased state (e.g., as cancerous cells, during pathogen infection, etc.). For example, engrafted genetically modified mice of the present disclosure can be used to identify novel therapeutics; or as another example, to identify agents that are toxic to human cells of the erythroid lineage, and to identify agents that prevent against, mitigate, or reverse the toxic effects of toxic agents on human cells of the erythroid lineage; etc. As another example, engrafted genetically modified animals of the present disclosure provide a useful system for predicting the responsiveness of an individual to a disease therapy, e.g., by providing an in vivo platform for screening the responsiveness of an individual's immune system to an agent, e.g., a therapeutic agent, to predict the responsiveness of an individual to that agent. As yet another example, hematopoietic stem and progenitor cells (HSPCs) from patients with genetic abnormalities in erythrocytes (e.g. sickle-cell anemia, beta-thalassemia, etc.) or iPS-derived HSPCs with such genetic modifications can be engrafted into HMOX-1-deficient HIS mice to model certain erythroid diseases and test potential therapeutics.

As one non-limiting example, engrafted genetically modified mice of the present disclosure find use in the generation of mouse models of pathogen infection by parasites that target human erythroid cells, e.g., Plasmodium, Babesia, Theileria, and the like. Such mouse models of infection will be useful in both research, e.g., to better understand the progression of infection in humans, and in drug discovery, e.g., to identify candidate agents that protect against or treat infection by such parasites.

Protozoans of the genus Plasmodium are the cause of malaria in humans. Malaria begins with a bite from an infected Anopheles mosquito, which introduces the protozoa via its saliva into the circulatory system, and ultimately to the liver where they mature and reproduce. The protozoa then enter the bloodstream and infect cells of the erythroid lineage at various stages of maturation.

Five species of Plasmodium can infect and be transmitted by humans. The vast majority of deaths are caused by P. falciparum, while P. vivax, P. ovale, and P. malariae cause a generally milder form of malaria that is rarely fatal. This believed to be due at least in part to the type(s) of cells targeted by each species: P. falciparum grows in red blood cells (RBCs) of all maturities whereas, for example, P. vivax is restricted to growth in reticulocytes, which represent only approximately 1%-2% of total RBCs in the periphery. In addition, P. falciparum causes severe malaria via a distinctive property not shared by any other human malaria, namely, that of sequestration. Within the 48-hour asexual blood stage cycle, the mature forms change the surface properties of infected red blood cells, causing them to stick to blood vessels (a process called cyto adherence). This leads to obstruction of the microcirculation and results in dysfunction of multiple organs.

Symptoms of malaria include fever, chills, headache, sweats, fatigue, anemia, nausea, dry (nonproductive) cough, muscle and/or back pain, and an enlarged spleen. Other symptoms and complications associated with malaria include brain infection (cerebritis), hemolytic anemia, kidney failure, liver failure, meningitis, pulmonary edema, and hemorrhaging from the spleen. Generally, an individual at risk for developing malaria will begin to show symptoms 7 days or more after infection, e.g., 9 to 14 days after the initial infection by P. falciparum, 12 to 18 days after the initial infection by P. vivax or P. ovale, 18 to 40 days after the initial infection by P. malariae, or 11 to 12 days after the initial infection by P. knowlesi. Anti-malaria agents used in the art to treat or prevent malaria include chloroquine, quinidine, doxycycline, tetracycline, clindamycin, atovaquone plus proguanil (Malarone), Mefloquine, artesunate, and pyrimethamine plus sulfadoxine (Fansidar).

Methods for determining if a subject has been infected with Plasmodium are well known in the art, and include, for example, microscopic examination of blood using blood films, with antigen-based Rapid Diagnostic Tests (RDT), e g, immunochromatography-based RDTs, by detection of parasite DNA by polymerase chain reaction (PCR), etc. Any convenient method may be used to determine if the human red blood cells of the subject have been infected with the pathogen.

The presence of human red blood cells in the peripheral blood of HSC-engrafted HMOX-1^{-/-} human immune system (HIS) mouse models offers a unique opportunity for modeling malaria with in vivo mouse models. Malaria begins a life cycle stage after infection where sporozoites injected from a mosquito bite initially infect the liver and then emerging merozoites infect human RBCs (Vaughan et al. (2012) J Clin Invest 122(10):3618-28; Minkah et al (2018) Front Immunol. 9:807; Kaushansky et al. (2014) Cell Microbiol. 16(5):602-11; Good et al. (2015) Trends Parasitol. 31(11):583-94; Foquet et al. (2017) Methods Mol Biol. 1506:117-30). Whereas human liver infection has been modeled in vivo with immune-deficient mice that can be engrafted with human hepatocytes, e.g., in mice with deletion of fumarylacetoacetate hydrolase (FAH) that allows for ablation of mouse liver and replacement with human liver, in vivo studies in mouse of malaria have been hampered by the lack of survival of human RBCs in mice (Vaughan et al. (2012) J Clin Invest 122(10):3618-28). By combining HMOX-1-deficient HIS models with modifications that allow human liver engraftment, a superior model is generated for studying the complete malaria life cycle and testing potential treatments at all stages of malaria.

Thus, in one embodiment, the HMOX-1-deficient MSRG47 mice or rats described herein are bred to FAH−/− SRG model (FSRG) (see, e.g., Carbonaro et al. (2023) Sci. Adv. 9, eadf4490; Carbonaro et al. (2022) Sci. Rep. 12:14079, both incorporated herein by reference) to generate a human immune system model that allows for human RBCs, human leukocytes, and human hepatocytes to develop (e.g., after human HSC and human hepatocyte engraftment). Such animals can be utilized to test potential malaria therapies, especially immune-based therapies, and are envisioned in the methods for screening potential therapies described herein.

Another example of pathogens of interest are protozoans of the genus Babesia. Babesia infection results in a malaria-like disease called babesiosis. Babesiosis is a vector-borne illness usually transmitted by Ixodes scapularis ticks. The disease is typically caused by B. microti in humans, B. canis rossi and B. canis canis in dogs, B. bovis in cows, and B. bigemina in cattle. Babesia microti, which infects humans, uses the same tick vector as Lyme disease and ehrlichiosis, and may occur in conjunction with these other diseases. The protozoa can also be transmitted by blood transfusion.

In humans, babesiosis may be asymptomatic, or characterized by symptoms ranging from mild fever and diarrhea to high fever, shaking chills, and severe anemia. In severe cases, organ failure, including respiratory distress syndrome, may occur. Severe cases occur mostly in people who have had a splenectomy, or persons with an immunodeficiency, such as HIV/AIDS patients. Treatment typically comprises a two-drug regimen of quinine and clindamycin, or of atovaquone and azithromycin. In instances where babesiosis appears life-threatening, a blood exchange transfusion is performed, in which infected red blood cells are removed and replaced with uninfected ones.

Definitive diagnosis of infection by Babesia is by the identification of the parasite on a Giemsa-stained thin blood smear. The parasite appears in erythrocytes as paired merozoites forming the “Maltese cross formation” in humans or “two pears hanging together” in animals. Other diagnostic methods include PCR of peripheral blood, and serologic testing for antibodies (IgG, IgM) against Babesia.

Yet another malaria-like disease, theileriosis, is caused by protozoans of the genus Theileria. In humans, theileriosis is caused by T. microti; in horses, by T. equi (“Equine Piroplasmosis”); in sheep and goats, by T. lestoquardi; and in cattle, African buffalo, water buffalo, and water bucks, by T. annulata (“Tropical Theileriosis”, also known as “Mediterranean theileriosis”) or T. parva (“East Coast fever”, also known as “Corridor disease”). Theirleriosis is transmitted to the host by various tick species including Ixodes scapularis, Rhipicephalus, Dermacentor, Haemaphysalis, and Hyalomma. The organism reproduces in the tick as it progresses through its life stage, and matures and enters the saliva after the tick attaches to a host. Usually, the tick must be attached for a few days before it becomes infective. However, if environmental temperatures are high, infective sporozoites can develop in ticks on the ground, and may enter the host within hours of attachment.

Theirleriosis in humans typically presents as fever and hemolysis. Definitive diagnosis of infection by Theileria is by the identification of the parasite on a Giemsa-stained thin blood smear.

Engrafted genetically modified animals of the present disclosure find use in screening candidate agents to identify those that will prevent (e.g., vaccines) or treat infections by Plasmodium, Babesia, Theileria, and other parasites that target human erythrocytes. The terms “treatment”, “treating” and the like are used herein to generally include obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein include any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Candidate agents of interest as anti-parasitic therapeutics include those that may be administered before, during or after the infection with the parasite, and which, when administered in an effective amount, inhibit the effects of a parasite on an individual (i.e., the host), for example, by killing the parasite or the cell infected by the parasite, by preventing the propagation of the parasite, by preventing the production or action of an agent produced by the parasite that is toxic to the individual (i.e., a toxin), etc. The terms “individual,” “subject,” “host,” and “patient,” are used interchangeably herein and include any mammalian subject for whom diagnosis, treatment, or therapy is desired, particularly humans.

In screening assays for biologically active agents, a human hematopoietic cell-engrafted genetically modified non-human animal of the present disclosure, is contacted with a candidate agent of interest and the effect of the candidate agent is assessed by monitoring one or more output parameters. These output parameters may be reflective of the viability of the human red blood cells, e.g., the total number of human red blood cells; or of the apoptotic state of the human red blood cells, e.g., the amount of cell blebbing, the amount of phosphatidylserine on the human red blood cell surface, and the like, by methods that are well known in the art. Alternatively or additionally, the output parameters may be reflective of the effect of the agent on infection in the human red blood cells of the human hematopoietic cell-engrafted genetically modified non-human animal of the present disclosure.

Candidate agents for screening or methods of the present disclosure may include, for examples, organic molecules (e.g., small molecule inhibitors), nucleic acids (e.g., RNA interfering agents, oligonucleotides, or nucleic acids that encode polypeptides), peptides, peptidomimetic inhibitors, aptamers, antibodies, intrabodies, etc. The “RNA interfering agent” used herein may be a small interfering RNA (siRNA), CRISPR RNA (crRNA), CRISPR guide RNA (gRNA), a small hairpin RNA (shRNA), a microRNA (miRNA), or a piwi-interacting RNA (piRNA).

Candidate agents are screened for biological activity by administering the agent to at least one and usually a plurality of samples, sometimes in conjunction with samples lacking the agent. The change in parameters in response to the agent is measured, and the result evaluated by comparison to reference samples, e.g., in the presence and absence of the agent, obtained with other agents, etc. In instances in which a screen is being performed to identify candidate agents that will prevent, mitigate or reverse the effects of a pathogen, the screen is typically performed in the presence of the pathogenic agent, where the pathogenic agent is added at the time most appropriate to the results to be determined. For example, in cases in which the protective/preventative ability of the candidate agent is tested, the candidate agent may be added before the pathogen, simultaneously with the pathogen, or subsequent to infection by the pathogen. As another example, in cases in which the ability of the candidate agent to reverse the effects of a pathogen is tested, the candidate agent may be added subsequent to infection with the pathogen. As mentioned above, in some instances, the “sample” is a genetically modified non-human animal that has been engrafted with cells, e.g., the candidate agent is provided to a genetically modified non-human animal that has been engrafted with human hematopoietic cells. In some instances, the “sample” is the human hematopoietic cells to be engrafted, i.e., the candidate agent is provided to cells, e.g., reticulocytes, erythrocytes, etc., prior to engraftment into the immunodeficient genetically modified animal.

If the candidate agent is to be administered directly to the engrafted genetically modified animal, the agent may be administered by any of a number of well-known methods in the art for the administration of peptides, small molecules and nucleic acids to mice. For example, the agent may be administered orally, mucosally, topically, intradermally, or by injection, e.g., intraperitoneal, subcutaneous, intramuscular, or intravenous injection, and the like. The agent may be administered in a buffer, or it may be incorporated into any of a variety of formulations, e.g., by combination with appropriate pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. The agent may be systemic after administration or may be localized by the use of regional administration, intramural administration, or use of an implant that acts to retain the active dose at the site of implantation. The active agent may be formulated for immediate activity or it may be formulated for sustained release. If the agent(s) are provided to cells prior to engraftment, the agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

An analysis of the response of cells in the engrafted genetically modified animal to the candidate agent may be performed at any time following treatment with the agent. For example, the cells may be analyzed 1, 2, or 3 days, sometimes 4, 5, or 6 days, sometimes 8, 9, or 10 days, sometimes 14 days, sometimes 21 days, sometimes 28 days, sometimes 1 month or more after contact with the candidate agent, e.g., 2 months, 4 months, 6 months or more. In some embodiments, the analysis comprises analysis at multiple time points. The selection of the time point(s) for analysis will be based upon the type of analysis to be performed, as will be readily understood by the ordinarily skilled artisan.

The analysis may comprise measuring any of the parameters described herein or known in the art for measuring cell viability, cell proliferation, cell identity, cell morphology, and cell function, particularly as they may pertain to cells of the immune cells. For example, flow cytometry may be used to determine the total number of hematopoietic cells or the number of cells of a particular hematopoietic cell type. Histochemistry or immunohistochemistry may be performed to determine the apoptotic state of the cells, e.g., terminal deoxynucleotidyl transferase dUTP nick end labeling (TUNEL) to measure DNA fragmentation, or immunohistochemistry to detect Annexin V binding to phosphatidylserine on the cell surface. Flow cytometry may also be employed to assess the proportions of differentiated cells and differentiated cell types, e.g., to determine the ability of hematopoietic cells to survive and/or differentiate in the presence of agent. ELISAs, Westerns, and Northern blots may be performed to determine the levels of cytokines, chemokines, immunoglobulins, etc. expressed in the engrafted genetically modified mice, e.g., to assess the function of the engrafted cells, to assess the survival of erythrocytes, etc. In vivo assays to test the function of immune cells, as well as assays relevant to particular diseases or disorders of interest such as anemia, e.g., sickle cell anemia, etc. may also be performed. See, e.g., Current Protocols in Immunology (Richard Coico, ed. John Wiley & Sons, Inc. 2012) and Immunology Methods Manual (I. Lefkovits ed., Academic Press 1997), the disclosures of which are incorporated herein by reference.

So, for example, a method is provided for determining the effect of an agent on erythroid cells infectable or infected by pathogen, comprising administering the agent to a genetically modified non-human animal of present disclosure, that has been engrafted with human reticulocytes and/or erythrocytes; measuring a parameter of the viability of the engrafted cells over time in the presence of the agent; and comparing that measurement to the measurement from an engrafted genetically modified non-human animal not exposed to the agent. The agent is determined to be anti-pathogenic if it reduces the infection of and/or the death of human erythrocytes in the peripheral blood of the mouse by at least 20%, at least 30%, at least 40%, at least 50%, at least 60%, at least 70%, at least 80%, at least 90%, or 100% (i.e., to undetectable amounts), following one or more administrations of the agent over a selected period of time. In a specific embodiment, the administration of the drug or combination of drugs is at least three days, at least one week, at least 10 days, at least two weeks, at least three weeks, at least four weeks, at least 6 weeks, at least 8 weeks, at least 10 weeks after engraftment with human hematopoietic cells.

Other examples of uses for the subject mice are provided elsewhere herein. Additional applications of the genetically modified and engrafted mice described in this disclosure will be apparent to those skilled in the art upon reading this disclosure.

### Additional Exemplary Embodiments

In exemplary embodiment 1, provided herein is a genetically modified non-human animal, comprising: (i) a homozygous null mutation in Rag2 gene; (ii) a homozygous null mutation in IL2rg gene; and (iii) a homozygous null mutation in the non-human animal Heme oxygenase-1 (Hmox-1) gene.

In exemplary embodiment 2, provided herein is a genetically modified non-human animal of embodiment 1, wherein a genetically modified non-human animal comprises a homozygous null mutation in Rag1 gene.

In exemplary embodiment 3, provided herein is a genetically modified non-human animal of embodiment 1 or 2, wherein the null mutation is a deletion of at least exons that correspond to mouse Hmox-1 exons 3-5.

In exemplary embodiment 4, provided herein is a genetically modified non-human animal of any one of embodiments 1-3, wherein the null mutation is a deletion of the full Hmox-1 endogenous coding sequence.

In exemplary embodiment 5, provided herein is a genetically modified non-human animal of any one of embodiments 1˜4 comprising a homozygous null mutation in Fah gene.

In exemplary embodiment 6, provided herein is a genetically modified non-human animal of embodiment 5, wherein the homozygous null mutation in Fah gene comprises an insertion, a deletion, and/or a substitution in the endogenous Fah gene.

In exemplary embodiment 7, provided herein is a genetically modified non-human animal of any one of embodiments 1-6, wherein the genetically modified non-human animal expresses a human or humanized SIRPA polypeptide encoded by a nucleic acid operably linked to a Sirpa promoter.

In exemplary embodiment 8, provided herein is a genetically modified non-human animal of embodiment 7, wherein the genetically modified non-human animal comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In exemplary embodiment 9, provided herein is a genetically modified non-human animal of embodiment 8, wherein the Sirpa gene comprises exons 2-4 of a human SIRPA gene.

In exemplary embodiment 10, provided herein is a genetically modified non-human animal of embodiment 8 or 9, wherein the genetically modified non-human animal expresses a Sirpa polypeptide comprising an extracellular portion of a human SIRPA polypeptide and an intracellular portion of a non-human animal Sirpa polypeptide.

In exemplary embodiment 11, provided herein is a genetically modified non-human animal of any one of embodiments 8-10, wherein the non-human animal Sirpa polypeptide is an endogenous non-human animal Sirpa polypeptide, and/or the non-human animal Sirpa gene is an endogenous non-human animal gene.

In exemplary embodiment 12, provided herein is a genetically modified non-human animal of embodiment 7, wherein the genetically modified non-human animal expresses a human SIRPA polypeptide encoded by a nucleic acid operably linked to a Sirpa promoter.

In exemplary embodiment 13, provided herein is a genetically modified non-human animal of any one of embodiments 7-12, wherein the genetically modified non-human animal further expresses one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to an EPO promoter.

In exemplary embodiment 14, provided herein is a genetically modified non-human animal of any one of embodiments 7-13, wherein at least one promoter operably linked to a nucleic acid that encodes a human or humanized protein is an endogenous non-human animal promoter.

In exemplary embodiment 15, provided herein is a genetically modified non-human animal of embodiment 14, wherein all promoters operably linked to the nucleic acids that encode the human or humanized proteins are endogenous non-human animal promoters.

In exemplary embodiment 16, provided herein is a genetically modified non-human animal of embodiment 14 or 15, wherein the endogenous non-human animal promoter is at the corresponding non-human animal gene locus.

In exemplary embodiment 17, provided herein is a genetically modified non-human animal of any one of embodiments 7-16, comprising a null mutation in at least one corresponding non-human animal gene at the corresponding non-human animal gene locus.

In exemplary embodiment 18, provided herein is a genetically modified non-human animal of any one of embodiments 7-17, wherein the genetically modified non-human animal is heterozygous for at least one allele comprising the nucleic acid sequence that encodes the human or humanized protein.

In exemplary embodiment 19, provided herein is a genetically modified non-human animal of any one of embodiments 7-17, wherein the genetically modified non-human animal is homozygous for at least one allele comprising the nucleic acid sequence that encodes the human or humanized protein.

In exemplary embodiment 20, provided herein is a genetically modified non-human animal of any one of embodiments 7-19, wherein the genetically modified non-human animal expresses a human M-CSF protein encoded by a nucleic acid operably linked to an M-CSF promoter.

In exemplary embodiment 21, provided herein is a genetically modified non-human animal of any one of embodiments 7-20, wherein the genetically modified non-human animal expresses a human or humanized CD47 protein encoded by a nucleic acid operably linked to a CD47 promoter.

In exemplary embodiment 22, provided herein is a genetically modified non-human animal of embodiment 21, wherein the genetically modified non-human animal expresses a humanized CD47 protein, and the humanized CD47 protein comprises an extracellular portion of a human CD47 protein and an intracellular portion of an endogenous non-human animal CD47 protein.

In exemplary embodiment 23, provided herein is a genetically modified non-human animal of any one of embodiments 7-22, wherein the genetically modified non-human animal expresses: (i) a human or humanized SIRPA protein encoded by a nucleic acid operably linked to a Sirpa promoter; (ii) a human M-CSF protein encoded by a nucleic acid operably linked to an M-CSF promoter; and (iii) a human or humanized CD47 protein encoded by a nucleic acid operably linked to a CD47 promoter.

In exemplary embodiment 24, provided herein is a genetically modified non-human animal of any one of embodiments 7-23, wherein the genetically modified non-human animal expresses a human EPO protein encoded by a nucleic acid operably linked to an EPO promoter.

In exemplary embodiment 25, provided herein is a genetically modified non-human animal of any one of embodiments 1-24, further comprising an engraftment of human hematopoietic cells.

In exemplary embodiment 26, provided herein is a genetically modified non-human animal of embodiment 25, wherein human hematopoietic cells comprise one or more cells selected from the group consisting of a human CD34-positive cell, a human hematopoietic stem cell, a human hematopoietic progenitor cell, a human erythroid precursor cell, and a human erythrocyte.

In exemplary embodiment 27, provided herein is a genetically modified non-human animal of embodiment 36, wherein the animal comprises human cells of erythroid lineage.

In exemplary embodiment 28, provided herein is a genetically modified non-human animal of embodiment 27, wherein the non-human animal further comprises an infection with a pathogen that targets human cells of the erythroid lineage.

In exemplary embodiment 29, provided herein is a genetically modified non-human animal of any one of embodiments 25-28, wherein the animal comprises the inactivated endogenous FAH gene, and wherein the animal further comprises transplanted human hepatocytes.

In exemplary embodiment 30, provided herein is a genetically modified non-human animal of embodiment 28 or embodiment 29, wherein the pathogen can cause malaria in human.

In exemplary embodiment 31, provided herein is a genetically modified non-human animal of embodiment 28, wherein the pathogen is selected from a Plasmodium sp., Babesia sp., and a Theileri sp.

In exemplary embodiment 32, provided herein is a genetically modified non-human animal of any one of embodiments 25-27, wherein the engrafted human hematopoietic cells give rise to abnormal human cells of the erythroid lineage.

In exemplary embodiment 33, provided herein is a genetically modified non-human animal of embodiment 32, wherein the engrafted human hematopoietic cells comprise a mutation in β-globin gene that leads to sickle cell disease.

In exemplary embodiment 34, provided herein is a genetically modified non-human animal of any one of embodiments 1-33, wherein the genetically modified non-human animal is a mammal

In exemplary embodiment 35, provided herein is a genetically modified non-human animal of embodiment 34, wherein the mammal is a rodent, such as a rat or a mouse.

In exemplary embodiment 36, provided herein is a genetically modified non-human animal of embodiment 35, wherein the rodent is a mouse.

In exemplary embodiment 37, provided herein is a method for identifying an agent that inhibits an infection by a pathogen that targets human cells of the erythroid lineage, the method comprising: a. administering the agent to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; iii. an engraftment of human hematopoietic cells; and iv. an infection by a pathogen that targets human cells of the erythroid lineage, and b. determining whether the agent reduces the amount of the pathogen and/or inhibits the activity of the pathogen in the pathogen-infected non-human animal.

In exemplary embodiment 38, provided herein is a method for identifying an agent that prevents an infection by a pathogen that targets human cells of the erythroid lineage, the method comprising: a. administering the agent to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic cells, b. injecting the genetically modified non-human animal with parasitized reticulocytes or erythrocytes, and c. determining whether the agent prevents the infection of the human reticulocytes and/or erythrocytes of the non-human animal.

In exemplary embodiment 39, provided herein is a method of embodiment 37 or 38, wherein the pathogen can cause malaria in human.

In exemplary embodiment 40, provided herein is a method of embodiment 37 or 38, wherein the pathogen is selected from a Plasmodium sp., Babesia sp., and a Theileri sp.

In exemplary embodiment 41, a method for identifying an agent that treats sickle cell disease, the method comprising: a. administering the agent to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic cells comprising a mutation in β-globin gene that leads to sickle cell disease, and b. determining whether the agent prevents or reduces red cell sickling in the non-human animal.

In exemplary embodiment 42, provided herein is a method for assessing therapeutic efficacy of a drug candidate targeting human red blood cells, the method comprising: a. administering the drug candidate to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic progenitor cells, and b. monitoring the human red blood cells in the non-human animal to assess the therapeutic efficacy of the drug candidate.

In exemplary embodiment 43, provided herein is a method of embodiment 42, wherein the human red blood cells are monitored to determine whether generation and/or survival of the human red blood cells in the non-human animal is increased by the drug candidate.

In exemplary embodiment 44, provided herein is a method of assessing toxicity of a drug candidate on human red blood cells, comprising: a. administering the drug candidate to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic progenitor cells, and b. monitoring the human red blood cells in the non-human animal to assess the toxicity of the drug candidate.

In exemplary embodiment 45, provided herein is a method of embodiment 44, wherein the toxicity is on-target toxicity or off-target toxicity.

In exemplary embodiment 46, provided herein is a method of embodiment 44 or 45, wherein the human red blood cells are monitored to determine whether number of the human red blood cells in the non-human animal is reduced by the drug candidate.

In exemplary embodiment 47, provided herein is a method of embodiment 46, wherein the drug candidate is a chemotherapeutic agent, or an anti-malaria agent.

In exemplary embodiment 48, provided herein is a method of embodiment 44 or 45, wherein the human red blood cells are monitored to assess whether the drug candidate induces agglutination of the red blood cells.

In exemplary embodiment 49, provided herein is a method of embodiment 48, wherein the drug candidate is a modulator of a human CD47 protein.

In exemplary embodiment 50, provided herein is a method of embodiment 49, wherein the modulator is an antibody.

In exemplary embodiment 51, provided herein is a method of identifying an agent that reduces toxicity of a toxic drug on human red blood cells, comprising: a. administering the agent and the toxic drug to a genetically modified non-human animal, wherein the genetically modified non-human animal comprises: i. a homozygous null mutation in the non-human animal Hmox-1 gene; ii. a homozygous null mutation in Rag2 gene and a homozygous null mutation in IL2rg gene; and iii. an engraftment of human hematopoietic progenitor cells, and b. determining whether the agent reduces the toxicity of the toxic drug on human red blood cells in the non-human animal.

In exemplary embodiment 52, provided herein is a method of embodiment 51, wherein the agent and the toxic drug are administered to the non-human animal concurrently or sequentially.

In exemplary embodiment 53, provided herein is a method of any one of embodiments 37-52, wherein the genetically modified non-human animal comprises a homozygous null mutation in Rag1 gene.

In exemplary embodiment 54, provided herein is a method of any one of embodiments 37-53, wherein the null mutation is a deletion of at least exons that correspond to mouse Hmox-1 exons 3-5.

In exemplary embodiment 55, provided herein is a method of any one of embodiments 37-54, wherein the null mutation is a deletion of the full Hmox-1 endogenous coding sequence.

In exemplary embodiment 56a, provided herein is a method of any one of embodiments 37-55, wherein the genetically modified non-human animal comprises engraftment of human hepatocytes.

In exemplary embodiment 56b, provided herein is a method of any one of embodiments 37-56a, wherein the genetically modified non-human animal comprises a homozygous null mutation in Fah gene.

In exemplary embodiment 57, provided herein is a method of embodiment 56b, wherein the homozygous null mutation in Fah gene comprises an insertion, a deletion, and/or a substitution in the endogenous Fah gene.

In exemplary embodiment 58, provided herein is a method of any one of embodiments 37-57, wherein the genetically modified non-human animal expresses a human or humanized SIRPA polypeptide encoded by a nucleic acid operably linked to a Sirpa promoter.

In exemplary embodiment 59, provided herein is a method of embodiment 58, wherein the genetically modified non-human animal comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In exemplary embodiment 60, provided herein is a method of embodiment 59, wherein the Sirpa gene comprises exons 2-4 of a human SIRPA gene.

In exemplary embodiment 61, provided herein is a method of embodiment 59 or 60, wherein the genetically modified non-human animal expresses a Sirpa polypeptide. comprising an extracellular portion of a human SIRPA polypeptide and an intracellular portion of a non-human animal Sirpa polypeptide.

In exemplary embodiment 62, provided herein is a method of any one of embodiments 59-61, wherein the non-human animal Sirpa polypeptide is an endogenous non-human animal Sirpa polypeptide, and/or the non-human animal Sirpa gene is an endogenous non-human animal gene.

In exemplary embodiment 63, provided herein is a method of embodiment 58, wherein the genetically modified non-human animal expresses a human SIRPA polypeptide encoded by a nucleic acid operably linked to a Sirpa promoter.

In exemplary embodiment 64, provided herein is a method of any one of embodiments 58-63, wherein the genetically modified non-human animal further expresses one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to a EPO promoter.

In exemplary embodiment 65, provided herein is a method of any one of embodiments 58-64, wherein at least one promoter operably linked to a nucleic acid that encodes a human or humanized protein is an endogenous non-human animal promoter.

In exemplary embodiment 66, provided herein is a method of embodiment 65, wherein all promoters operably linked to the nucleic acids that encodes the human or humanized proteins are endogenous non-human animal promoters.

In exemplary embodiment 67, provided herein is a method of embodiment 65 or 66, wherein the endogenous non-human animal promoter is at the corresponding non-human animal gene locus.

In exemplary embodiment 68, provided herein is a method of any one of embodiments 58-67, the genetically modified non-human animal comprises a null mutation in at least one corresponding non-human animal gene at the corresponding non-human animal gene locus.

In exemplary embodiment 69, provided herein is a method of any one of embodiments 58-68, wherein the genetically modified non-human animal is heterozygous for at least one allele comprising the nucleic acid sequence that encodes the human or humanized protein.

In exemplary embodiment 70, provided herein is a method of any one of embodiments 58-69, wherein the genetically modified non-human animal is homozygous for at least one allele comprising the nucleic acid sequence that encodes the human or humanized protein.

In exemplary embodiment 71, provided herein is a method of any one of embodiments 58-70 wherein the genetically modified non-human animal expresses a human M-CSF protein encoded by a nucleic acid operably linked to an M-CSF promoter.

In exemplary embodiment 72, provided herein is a method of any one of embodiments 58-71, wherein the genetically modified non-human animal expresses a human or humanized CD47 protein encoded by a nucleic acid operably linked to a CD47 promoter.

In exemplary embodiment 73, provided herein is a method of embodiment 72, wherein the genetically modified non-human animal expresses a humanized CD47 protein, and the humanized CD47 protein comprises an extracellular portion of a human CD47 protein and an intracellular portion of an endogenous non-human animal CD47 protein.

In exemplary embodiment 74, provided herein is a method of any one of embodiments 58-73, wherein the genetically modified non-human animal expresses: (i) a human or humanized SIRPA protein encoded by a nucleic acid operably linked to a Sirpa promoter; (ii) a human M-CSF protein encoded by a nucleic acid operably linked to an M-CSF promoter; and (iii) a human or humanized CD47 protein encoded by a nucleic acid operably linked to a CD47 promoter.

In exemplary embodiment 75, provided herein is a method of any one of embodiments 58-74, wherein the genetically modified non-human animal expresses a human EPO protein encoded by a nucleic acid operably linked to an EPO promoter.

In exemplary embodiment 76, provided herein is a method of any one of embodiments 37-75, wherein the genetically modified non-human animal is a mammal

In exemplary embodiment 77, provided herein is a method of embodiment 76, wherein the mammal is a rodent, such as a rat or a mouse.

In exemplary embodiment 78, provided herein is a method of embodiment 77, wherein the rodent is a mouse.

In exemplary embodiment 79, provided herein is a genetically modified non-human animal cell, comprising: (i) a homozygous null mutation in Rag2 gene; (ii) a homozygous null mutation in IL2rg gene; and (iii) a homozygous null mutation in the non-human animal Heme oxygenase-1 (Hmox-1) gene.

In exemplary embodiment 80, provided herein is a genetically modified non-human animal cell of embodiment 79 comprising a homozygous null mutation in Rag1 gene.

In exemplary embodiment 81, provided herein is a genetically modified non-human animal cell of embodiment 79 or 80, wherein the null mutation is a deletion of at least exons that correspond to mouse Hmox-1 exons 3-5.

In exemplary embodiment 82, provided herein is a genetically modified non-human animal cell of any one of embodiments 79-81, wherein the null mutation is a deletion of the full Hmox-1 endogenous coding sequence.

In exemplary embodiment 83, provided herein is a genetically modified non-human animal cell of any one of embodiments 79-82 comprising a homozygous null mutation in Fah gene.

In exemplary embodiment 84, provided herein is a genetically modified non-human animal cell of embodiment 83, wherein the homozygous null mutation in Fah gene comprises an insertion, a deletion, and/or a substitution in the endogenous Fah gene.

In exemplary embodiment 85, provided herein is a genetically modified non-human animal cell of any one of embodiments 79-84, wherein the genetically modified non-human animal cell expresses a human or humanized SIRPA polypeptide encoded by a nucleic acid operably linked to a Sirpa promoter.

In exemplary embodiment 86, provided herein is a genetically modified non-human animal cell of embodiment 85, wherein the genetically modified non-human animal cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In exemplary embodiment 87, provided herein is a genetically modified non-human animal cell of embodiment 86, wherein the Sirpa gene comprises exons 2-4 of a human SIRPA gene.

In exemplary embodiment 88, provided herein is a genetically modified non-human animal cell of embodiment 86 or 87, wherein the genetically modified non-human animal cell expresses a Sirpa polypeptide comprising an extracellular portion of a human SIRPA polypeptide and an intracellular portion of a non-human animal Sirpa polypeptide.

In exemplary embodiment 89, provided herein is a genetically modified non-human animal cell of any one of embodiments 86-88, wherein the non-human animal Sirpa polypeptide is an endogenous non-human animal Sirpa polypeptide, and/or the non-human animal Sirpa gene is an endogenous non-human animal gene.

In exemplary embodiment 90, provided herein is a genetically modified non-human animal cell of embodiment 85, wherein the genetically modified non-human animal cell expresses a human SIRPA polypeptide encoded by a nucleic acid operably linked to a Sirpa promoter.

In exemplary embodiment 91, provided herein is a genetically modified non-human animal cell of any one of embodiments 85-90, wherein the genetically modified non-human animal cell further expresses one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to an EPO promoter.

In exemplary embodiment 92, provided herein is a genetically modified non-human animal cell of any one of embodiments 85-91, wherein at least one promoter operably linked to a nucleic acid that encodes a human or humanized protein is an endogenous non-human animal promoter.

In exemplary embodiment 93, provided herein is a genetically modified non-human animal cell of embodiment 92, wherein all promoters operably linked to the nucleic acids that encode the human or humanized proteins are endogenous non-human animal promoters.

In exemplary embodiment 94, provided herein is a genetically modified non-human animal cell of embodiment 92 or 93, wherein the endogenous non-human animal promoter is at the corresponding non-human animal gene locus.

In exemplary embodiment 95, provided herein is a genetically modified non-human animal cell of any one of embodiments 85-94, comprising a null mutation in at least one corresponding non-human animal gene at the corresponding non-human animal gene locus.

In exemplary embodiment 96, provided herein is a genetically modified non-human animal cell of any one of embodiments 85-95, wherein the genetically modified non-human animal cell is heterozygous for at least one allele comprising the nucleic acid sequence that encodes the human or humanized protein.

In exemplary embodiment 97, provided herein is a genetically modified non-human animal cell of any one of embodiments 85-95, wherein the genetically modified non-human animal cell is homozygous for at least one allele comprising the nucleic acid sequence that encodes the human or humanized protein.

In exemplary embodiment 98, provided herein is a genetically modified non-human animal cell of any one of embodiments 85-97, wherein the genetically modified non-human animal cell expresses a human M-CSF protein encoded by a nucleic acid operably linked to an M-CSF promoter.

In exemplary embodiment 99, provided herein is a genetically modified non-human animal cell of any one of embodiments 85-98, wherein the genetically modified non-human animal cell expresses a human or humanized CD47 protein encoded by a nucleic acid operably linked to a CD47 promoter.

In exemplary embodiment 100, provided herein is a genetically modified non-human animal cell of embodiment 99, wherein the genetically modified non-human animal cell expresses a humanized CD47 protein, and the humanized CD47 protein comprises an extracellular portion of a human CD47 protein and an intracellular portion of an endogenous non-human animal CD47 protein.

In exemplary embodiment 101, provided herein is a genetically modified non-human animal cell of any one of embodiments 85-100, wherein the genetically modified non-human animal cell expresses: (i) a human or humanized SIRPA protein encoded by a nucleic acid operably linked to a Sirpa promoter; (ii) a human M-CSF protein encoded by a nucleic acid operably linked to an M-CSF promoter; and (iii) a human or humanized CD47 protein encoded by a nucleic acid operably linked to a CD47 promoter.

In exemplary embodiment 102, provided herein is a genetically modified non-human animal cell of any one of embodiments 85-101, wherein the genetically modified non-human animal cell expresses a human EPO protein encoded by a nucleic acid operably linked to an EPO promoter.

In exemplary embodiment 103, provided herein is a genetically modified non-human animal cell of any one of embodiments 79-102, wherein the genetically modified non-human animal cell is a mammalian cell.

In exemplary embodiment 104, provided herein is a genetically modified non-human animal cell of embodiment 103, wherein the mammalian cell is a rodent cell, such as a rat cell or a mouse cell.

In exemplary embodiment 105, provided herein is a genetically modified non-human animal cell of embodiment 104, wherein the rodent cell is a mouse cell.

In exemplary embodiment 106, provided herein is a genetically modified non-human animal cell of any one of embodiments 79-84, 86-87, 89, 92-97, and 103-105, wherein the genetically modified non-human animal cell is a non-human animal embryonic stem (ES) cell.

In exemplary embodiment 107, provided herein is a non-human animal embryonic stem cell, comprising in its genome: (i) a homozygous null mutation in Rag2 gene; (ii) a homozygous null mutation in IL2rg gene; and (iii) a homozygous null mutation in the non-human animal Heme oxygenase-1 (Hmox-1) gene.

In exemplary embodiment 108, provided herein is a non-human animal embryonic stem cell of embodiment 107, wherein the non-human animal embryonic stem cell comprises in its genome a homozygous null mutation in Rag1 gene.

In exemplary embodiment 109, provided herein is a non-human animal embryonic stem cell of embodiment 107 or 108, wherein the null mutation is a deletion of at least exons that correspond to mouse Hmox-1 exons 3-5.

In exemplary embodiment 110, provided herein is a non-human animal embryonic stem cell of any one of embodiments 107-109, wherein the null mutation is a deletion of the full Hmox-1 endogenous coding sequence.

In exemplary embodiment 111, provided herein is a non-human animal embryonic stem cell of any one of embodiments 107-110, wherein the non-human animal embryonic stem cell comprises in its genome a homozygous null mutation in Fah gene.

In exemplary embodiment 112, provided herein is a non-human animal embryonic stem cell of embodiment 111, wherein the homozygous null mutation in Fah gene comprises an insertion, a deletion, and/or a substitution in the endogenous Fah gene.

In exemplary embodiment 113, provided herein is a non-human animal embryonic stem cell of any one of embodiments 107-112, wherein the non-human animal embryonic stem cell comprises in its genome a nucleic acid that encodes a human or humanized SIRPA polypeptide, and wherein the nucleic acid is operably linked to a Sirpa promoter.

In exemplary embodiment 114, provided herein is a non-human animal embryonic stem cell of embodiment 113, wherein the genetically modified non-human animal embryonic stem cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In exemplary embodiment 115, provided herein is a non-human animal embryonic stem cell of embodiment 114, wherein the Sirpa gene comprises exons 2-4 of a human SIRPA gene.

In exemplary embodiment 116, provided herein is a non-human animal embryonic stem cell of any one of embodiments 114-115, wherein the non-human animal Sirpa polypeptide is an endogenous non-human animal Sirpa polypeptide.

In exemplary embodiment 117, provided herein is a non-human animal embryonic stem cell of any one of embodiments 114-115, wherein the non-human animal Sirpa gene is an endogenous non-human animal gene.

In exemplary embodiment 118, provided herein is a non-human animal embryonic stem cell of embodiment 113, wherein the genetically modified non-human animal embryonic stem cell comprises a nucleic acid encodes a human SIRPA polypeptide, and the nucleic acid is operably linked to a Sirpa promoter.

In exemplary embodiment 119, provided herein is a non-human animal embryonic stem cell of any one of embodiments 113-118, wherein the genetically modified non-human animal embryonic stem cell further comprises in its genome one or more nucleic acids selected from the group consisting of: a nucleic acid encoding a human TPO protein and operably linked to a TPO promoter; a nucleic acid encoding a human GM-CSF protein and operably linked to a GM-CSF promoter; a nucleic acid encoding a human IL3 protein and operably linked to a IL3 promoter; a nucleic acid encoding a human IL15 protein and operably linked to a IL15 promoter; a nucleic acid encoding a human M-CSF protein and operably linked to an M-CSF promoter; a nucleic acid encoding a human or humanized CD47 protein and operably linked to a CD47 promoter; and a nucleic acid encoding a human EPO protein and operably linked to an EPO promoter.

In exemplary embodiment 120, provided herein is a non-human animal embryonic stem cell of any one of embodiments 113-119, wherein at least one promoter operably linked to a nucleic acid that encodes a human or humanized protein is an endogenous non-human animal promoter.

In exemplary embodiment 121, provided herein is a non-human animal embryonic stem cell of embodiment 120, wherein all promoters operably linked to the nucleic acids that encode the human or humanized proteins are endogenous non-human animal promoters.

In exemplary embodiment 122, provided herein is a non-human animal embryonic stem cell of embodiment 120 or 121, wherein the endogenous non-human animal promoter is at the corresponding non-human animal gene locus.

In exemplary embodiment 123, provided herein is a non-human animal embryonic stem cell of any one of embodiments 113-122, comprising a null mutation in at least one corresponding non-human animal gene at the corresponding non-human animal gene locus.

In exemplary embodiment 124, provided herein is a non-human animal embryonic stem cell of any one of embodiments 113-123, wherein the genetically modified non-human animal cell is heterozygous for at least one allele comprising the nucleic acid sequence that encodes the human or humanized protein.

In exemplary embodiment 125, provided herein is a non-human animal embryonic stem cell of any one of embodiments 113-123, wherein the genetically modified non-human animal cell is homozygous for at least one allele comprising the nucleic acid sequence that encodes the human or humanized protein.

In exemplary embodiment 126, provided herein is a non-human animal embryonic stem cell of any one of embodiments 113-125, wherein the genetically modified non-human animal embryonic stem cell comprises in its genome a nucleic acid encoding a human M-CSF protein and operably linked to an M-CSF promoter.

In exemplary embodiment 127, provided herein is a non-human animal embryonic stem cell of any one of embodiments 113-126, wherein the genetically modified non-human animal embryonic stem cell comprises in its genome a nucleic acid encoding a human or humanized CD47 protein and operably linked to a CD47 promoter.

In exemplary embodiment 128, provided herein is a non-human animal embryonic stem cell of embodiment 127, wherein the genetically modified non-human animal cell embryonic stem cell comprises in its genome a nucleic acid encoding a humanized CD47 protein, and the humanized CD47 protein comprises an extracellular portion of a human CD47 protein and an intracellular portion of an endogenous non-human animal CD47 protein.

In exemplary embodiment 129, provided herein is a non-human animal embryonic stem cell of any one of embodiments 113-128, wherein the genetically modified non-human animal embryonic stem cell comprises in its genome: (i) a nucleic acid encoding a human or humanized SIRPA protein and operably linked to a Sirpa promoter; (ii) a nucleic acid encoding a human M-CSF protein and operably linked to an M-CSF promoter; and (iii) a nucleic acid encoding a human or humanized CD47 protein and operably linked to a CD47 promoter.

In exemplary embodiment 130, provided herein is a non-human animal embryonic stem cell of any one of embodiments 113-129, wherein the genetically modified non-human animal embryonic stem cell comprises in its genome a nucleic acid encoding a human EPO protein and operably linked to an EPO promoter.

In exemplary embodiment 131, provided herein is a non-human animal embryonic stem cell of any one of embodiments 107-130, wherein the genetically modified non-human animal embryonic stem cell is a mammalian embryonic stem cell.

In exemplary embodiment 132, provided herein is a non-human animal embryonic stem cell of embodiment 131, wherein the mammalian embryonic stem cell is a rodent embryonic stem cell, such as a rat embryonic stem cell or a mouse embryonic stem cell.

In exemplary embodiment 133, provided herein is a non-human animal embryonic stem cell of embodiment 132, wherein the rodent embryonic stem cell is a mouse embryonic stem cell.

In exemplary embodiment 134, provided herein is a method of making a non-human animal embryonic stem cell, comprising genetically engineering the non-human animal embryonic stem cell so that the non-human animal embryonic stem cell has a genome that comprises: (i) a homozygous null mutation in Rag2 gene; (ii) a homozygous null mutation in IL2rg gene; and (iii) a homozygous null mutation in the non-human animal Heme oxygenase-1 (Hmox-1) gene.

In exemplary embodiment 135, provided herein is a method of embodiment 134, wherein the non-human animal embryonic stem cell is further engineered to comprise in its genome a homozygous null mutation in Rag1 gene.

In exemplary embodiment 136, provided herein is a method of embodiment 134 or 135, wherein the null mutation is a deletion of at least exons that correspond to mouse Hmox-1 exons 3-5.

In exemplary embodiment 137, provided herein is a method of any one of embodiments 134-136, wherein the null mutation is a deletion of the full Hmox-1 endogenous coding sequence.

In exemplary embodiment 138, provided herein is a method of any one of embodiments 134-137, wherein the non-human animal embryonic stem cell is further engineered to comprise in its genome a homozygous null mutation in Fah gene.

In exemplary embodiment 139, provided herein is a method of embodiment 138, wherein the homozygous null mutation in Fah gene comprises an insertion, a deletion, and/or a substitution in the endogenous Fah gene.

In exemplary embodiment 140, provided herein is a method of any one of embodiments 134-139, wherein the non-human animal embryonic stem cell is further engineered to comprise in its genome a nucleic acid that encodes a human or humanized SIRPA polypeptide, and wherein the nucleic acid is operably linked to a Sirpa promoter.

In exemplary embodiment 141, provided herein is a method of embodiment 140, wherein the genetically modified non-human animal embryonic stem cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In exemplary embodiment 142, provided herein is a method of embodiment 141, wherein the Sirpa gene comprises exons 2-4 of a human SIRPA gene.

In exemplary embodiment 143, provided herein is a method of embodiment 141 or 142, wherein the non-human animal Sirpa polypeptide is an endogenous non-human animal Sirpa polypeptide.

In exemplary embodiment 144, provided herein is a method of any one of embodiment 141 or 142, wherein the non-human animal Sirpa gene is an endogenous non-human animal gene.

In exemplary embodiment 145, provided herein is a method of embodiment 140, wherein the genetically modified non-human animal embryonic stem cell comprises a nucleic acid encodes a human SIRPA polypeptide, and the nucleic acid is operably linked to a Sirpa promoter.

In exemplary embodiment 146, provided herein is a method of any one of embodiments 140-145, wherein the genetically modified non-human animal embryonic stem cell is further engineered to comprise in its genome one or more nucleic acids selected from the group consisting of: a nucleic acid encoding a human TPO protein and operably linked to a TPO promoter; a nucleic acid encoding a human GM-CSF protein and operably linked to a GM-CSF promoter; a nucleic acid encoding a human IL3 protein and operably linked to a IL3 promoter; a nucleic acid encoding a human IL15 protein and operably linked to a IL15 promoter; a nucleic acid encoding a human M-CSF protein and operably linked to an M-CSF promoter; a nucleic acid encoding a human or humanized CD47 protein and operably linked to a CD47 promoter; and a nucleic acid encoding a human EPO protein and operably linked to an EPO promoter.

In exemplary embodiment 147, provided herein is a method of any one of embodiments 140-146, wherein at least one promoter operably linked to a nucleic acid that encodes a human or humanized protein is an endogenous non-human animal promoter.

In exemplary embodiment 148, provided herein is a method of embodiment 147, wherein all promoters operably linked to the nucleic acids that encode the human or humanized proteins are endogenous non-human animal promoters.

In exemplary embodiment 149, provided herein is a method of embodiment 147 or 148, wherein the endogenous non-human animal promoter is at the corresponding non-human animal gene locus.

In exemplary embodiment 150, provided herein is a method of any one of embodiments 140-149, comprising a null mutation in at least one corresponding non-human animal gene at the corresponding non-human animal gene locus.

In exemplary embodiment 151, provided herein is a method of any one of embodiments 140-150, wherein the genetically modified non-human animal cell is heterozygous for at least one allele comprising the nucleic acid sequence that encodes the human or humanized protein.

In exemplary embodiment 152, provided herein is a method of any one of embodiments 140-150, wherein the genetically modified non-human animal cell is homozygous for at least one allele comprising the nucleic acid sequence that encodes the human or humanized protein.

In exemplary embodiment 153, provided herein is a method of any one of embodiments 140-152, wherein the genetically modified non-human animal embryonic stem cell is engineered to comprise in its genome a nucleic acid encoding a human M-CSF protein and operably linked to an M-CSF promoter.

In exemplary embodiment 154, provided herein is a method of any one of embodiments 140-153, wherein the genetically modified non-human animal embryonic stem cell is engineered to comprise in its genome a nucleic acid encoding a human or humanized CD47 protein and operably linked to a CD47 promoter.

In exemplary embodiment 155, provided herein is a method of embodiment 154, wherein the genetically modified non-human animal cell embryonic stem cell is engineered to comprise in its genome a nucleic acid encoding a humanized CD47 protein, and the humanized CD47 protein comprises an extracellular portion of a human CD47 protein and an intracellular portion of an endogenous non-human animal CD47 protein.

In exemplary embodiment 156, provided herein is a method of any one of embodiments 140-155, wherein the genetically modified non-human animal embryonic stem cell is engineered to comprise in its genome: (i) a nucleic acid encoding a human or humanized SIRPA protein and operably linked to a Sirpa promoter; (ii) a nucleic acid encoding a human M-CSF protein and operably linked to an M-CSF promoter; and (iii) a nucleic acid encoding a human or humanized CD47 protein and operably linked to a CD47 promoter.

In exemplary embodiment 157, provided herein is a method of any one of embodiments 140-156, wherein the genetically modified non-human animal embryonic stem cell is engineered to comprise in its genome a nucleic acid encoding a human EPO protein and operably linked to an EPO promoter.

In exemplary embodiment 158, provided herein is a method of any one of embodiments 134-157, wherein the genetically modified non-human animal embryonic stem cell is a mammalian embryonic stem cell.

In exemplary embodiment 159, provided herein is a method of embodiment 158, wherein the mammalian embryonic stem cell is a rodent embryonic stem cell, such as a rat embryonic stem cell or a mouse embryonic stem cell.

In exemplary embodiment 160, provided herein is a method of embodiment 159, wherein the rodent embryonic stem cell is a mouse embryonic stem cell.

In exemplary embodiment 161, provided herein is a non-human animal embryo comprises the non-human animal embryonic stem cell of any one of embodiments 106-133, or the non-human animal embryonic stem cell made according to the method of any one of embodiments 134-160.

In exemplary embodiment 162, provided herein is a method of making a non-human animal comprising in its genome: (i) a homozygous null mutation in Rag2 gene; (ii) a homozygous null mutation in IL2rg gene; and (iii) a homozygous null mutation in the non-human animal Heme oxygenase-1 (Hmox-1) gene, the method comprising steps of: (a) obtaining a non-human animal embryonic stem cell of any one of embodiments 106-133, or the non-human animal embryonic stem cell made according to the method of any one of embodiments 134-160; and (b) creating a non-human animal using the non-human animal embryonic cell of (a).

In exemplary embodiment 163, provided herein is a method of making a non-human animal comprising in its genome: (i) a homozygous null mutation in Rag2 gene; (ii) a homozygous null mutation in IL2rg gene; and (iii) a homozygous null mutation in the non-human animal Heme oxygenase-1 (Hmox-1) gene, the method comprising modifying the genome of the non-human animal so that it comprises: (i) a homozygous null mutation in Rag2 gene; (ii) a homozygous null mutation in IL2rg gene; and (iii) a homozygous null mutation in the non-human animal Heme oxygenase-1 (Hmox-1) gene.

In exemplary embodiment 164, provided herein is a method of embodiment 163, wherein the genetically modified non-human animal is further engineered to comprise a homozygous null mutation in Rag1 gene.

In exemplary embodiment 165, provided herein is a method of embodiment 163 or 164, wherein the null mutation is a deletion of at least exons that correspond to mouse Hmox-1 exons 3-5.

In exemplary embodiment 166, provided herein is a method of any one of embodiments 163-165, wherein the null mutation is a deletion of the full Hmox-1 endogenous coding sequence.

In exemplary embodiment 167, provided herein is a method of any one of embodiments 163-166, wherein the genetically modified non-human animal is further engineered to comprise a homozygous null mutation in Fah gene.

In exemplary embodiment 168, provided herein is a method of embodiment 167, wherein the homozygous null mutation in Fah gene comprises an insertion, a deletion, and/or a substitution in the endogenous Fah gene.

In exemplary embodiment 169, provided herein is a method of any one of embodiments 163-168, wherein the genetically modified non-human animal is further engineered to express a human or humanized SIRPA polypeptide encoded by a nucleic acid operably linked to a Sirpa promoter.

In exemplary embodiment 170, provided herein is a method of embodiment 169, wherein the genetically modified non-human animal is engineered to comprise a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In exemplary embodiment 171, provided herein is a method of embodiment 170, wherein the Sirpa gene comprises exons 2-4 of a human SIRPA gene.

In exemplary embodiment 172, provided herein is a method of embodiment 170 or 171, wherein the genetically modified non-human animal expresses a Sirpa polypeptide comprising an extracellular portion of a human SIRPA polypeptide and an intracellular portion of a non-human animal Sirpa polypeptide.

In exemplary embodiment 173, provided herein is a method of any one of embodiments 170-172, wherein the non-human animal Sirpa polypeptide is an endogenous non-human animal Sirpa polypeptide, and/or the non-human animal Sirpa gene is an endogenous non-human animal gene.

In exemplary embodiment 174, provided herein is a method of embodiment 169, wherein the genetically modified non-human animal expresses a human SIRPA polypeptide encoded by a nucleic acid operably linked to a Sirpa promoter.

In exemplary embodiment 175, provided herein is a method of any one of embodiments 169-174, wherein the genetically modified non-human animal is further engineered to express one or more human or humanized proteins selected from the group consisting of: a human TPO protein encoded by a nucleic acid operably linked to a TPO promoter; a human GM-CSF protein encoded by a nucleic acid operably linked to a GM-CSF promoter; a human IL3 protein encoded by a nucleic acid operably linked to a IL3 promoter; a human IL15 protein encoded by a nucleic acid operably linked to a IL15 promoter; a human M-CSF protein encoded by a nucleic acid operably linked to an M-CSF promoter; a human or humanized CD47 protein encoded by a nucleic acid operably linked to a CD47 promoter; and a human EPO protein encoded by a nucleic acid operably linked to a EPO promoter.

In exemplary embodiment 176, provided herein is a method of any one of embodiments 169-175, wherein at least one promoter operably linked to a nucleic acid that encodes a human or humanized protein is an endogenous non-human animal promoter.

In exemplary embodiment 177, provided herein is a method of embodiment 176, wherein all promoters operably linked to the nucleic acids that encode the human or humanized proteins are endogenous non-human animal promoters.

In exemplary embodiment 178, provided herein is a method of embodiment 176 or 177, wherein the endogenous non-human animal promoter is at the corresponding non-human animal gene locus.

In exemplary embodiment 179, provided herein is a method of any one of embodiments 169-178, comprising a null mutation in at least one corresponding non-human animal gene at the corresponding non-human animal gene locus.

In exemplary embodiment 180, provided herein is a method of any one of embodiments 169-179, wherein the genetically modified non-human animal is heterozygous for at least one allele comprising the nucleic acid sequence that encodes the human or humanized protein.

In exemplary embodiment 181, provided herein is a method of any one of embodiments 169-180, wherein the genetically modified non-human animal is homozygous for at least one allele comprising the nucleic acid sequence that encodes the human or humanized protein.

In exemplary embodiment 182, provided herein is a method of any one of embodiments 169-181 wherein the genetically modified non-human animal expresses a human M-CSF protein encoded by a nucleic acid operably linked to an M-CSF promoter.

In exemplary embodiment 183, provided herein is a method of any one of embodiments 169-182, wherein the genetically modified non-human animal expresses a human or humanized CD47 protein encoded by a nucleic acid operably linked to a CD47 promoter.

In exemplary embodiment 184, provided herein is a method of embodiment 183, wherein the genetically modified non-human animal expresses a humanized CD47 protein, and the humanized CD47 protein comprises an extracellular portion of a human CD47 protein and an intracellular portion of an endogenous non-human animal CD47 protein.

In exemplary embodiment 185, provided herein is a method of any one of embodiments 169-184, wherein the genetically modified non-human animal expresses: (i) a human or humanized SIRPA protein encoded by a nucleic acid operably linked to a Sirpa promoter; (ii) a human M-CSF protein encoded by a nucleic acid operably linked to an M-CSF promoter; and (iii) a human or humanized CD47 protein encoded by a nucleic acid operably linked to a CD47 promoter.

In exemplary embodiment 186, provided herein is a method of any one of embodiments 169-185, wherein the genetically modified non-human animal expresses a human EPO protein encoded by a nucleic acid operably linked to an EPO promoter.

In exemplary embodiment 187, provided herein is a method of any one of embodiments 163-186, wherein the genetically modified non-human animal is a mammal

In exemplary embodiment 188, provided herein is a method of embodiment 187, wherein the mammal is a rodent, such as a rat or a mouse.

In exemplary embodiment 189, provided herein is a method of embodiment 188, wherein the rodent is a mouse.

### EXAMPLES

The following Examples and the accompanying Drawings are provided so as to describe to those of ordinary skill in the art how to make and use methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. The Examples do not include detailed descriptions of conventional methods that would be well known to those of ordinary skill in the art (molecular cloning techniques, etc.). Unless indicated otherwise, parts are parts by weight, molecular weight is average molecular weight, temperature is indicated in Celsius, and pressure is at or near atmospheric.

### Example 1: Generation of Hmox-1 Knock Out Mouse

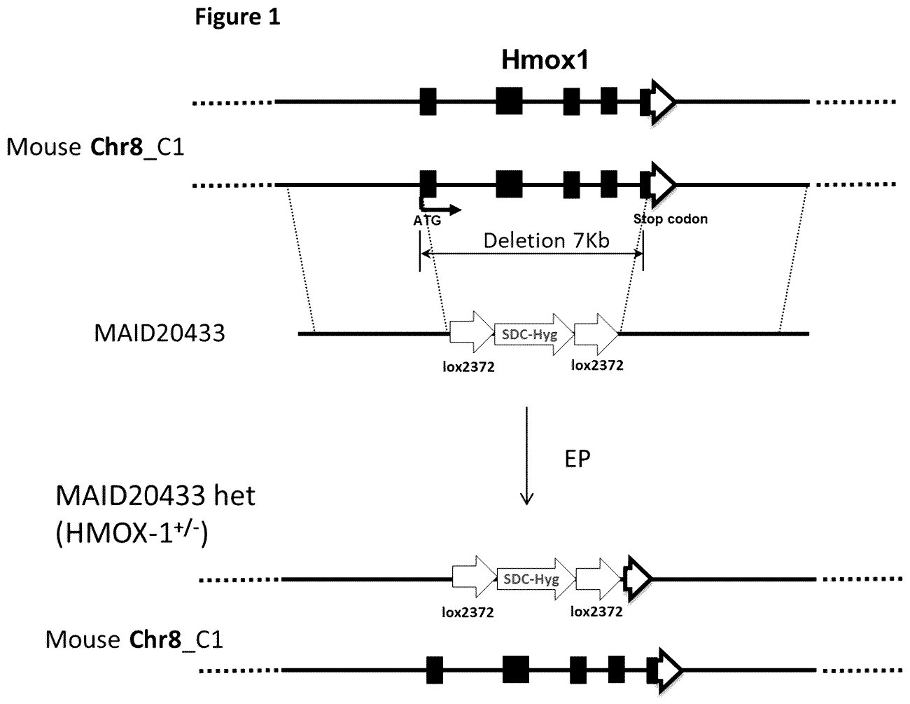

Mouse Hmox-1 gene was deleted in the mouse genome using VELOCIGENE® technology (see, e.g., U.S. Pat. No. 6,586,251 and Valenzuela et al. (2003) High-throughput engineering of the mouse genome coupled with high-resolution expression analysis. Nat. Biotech. 21(6): 652-659, both incorporated herein by reference). A 7 kb mouse genomic sequence of the mouse Hmox-1 gene, from the start codon ATG to the stop codon, was deleted on mouse chromosome 8 Cl, between coordinates chr8: 75093750-75100019 (GRCm38 assembly). FIG. 1.

In detail, mouse homology arms were made by PCR amplification using BAC clone RP23-102124 as the template, and are indicated in Table 1 below:

TABLE 1

| | | | Coordinates |
|---|---|---|---|
| Homology | | | (GRCm38 |
| Arm | 5′ primer | 3′ primer | assembly) |
| 5′; | GATGTTGCAACAGCAGCGA | CACCGGACTGGGCTAGTT | chr8:75093530- |
| 220 bp | GAA (SEQ ID NO. 6) | CA (SEQ ID NO. 7) | 75093749 |
| 3′; | ATGCAATACTGGCCCCCAG | GATTTGGGGCTGCTGGTTT | chr8:75100020- |
| 232 bp | G (SEQ ID NO. 8) | CAA (SEQ ID NO. 9) | 75100251 |

To make the targeting vector (designated MAID20433) from mouse BAC clone RP23-102124, a Hygromycin (Hyg) resistance self-deleting cassette (with CRE recombinase controlled by Protamine promoter) flanked by mutant lox sites (lox2372-hyg-lox2372), replaced ˜7 kb mouse sequence containing the mouse Hmox-1 gene by bacterial homologous recombination (BHR).

The final targeting vector contained from 5′ to 3′: the chloramphenicol resistance cassette (CM; not depicted on FIG. 1), the 5′ mouse homology arm, the lox2372-Hyg-lox2372 self-deleting cassette, the 3′ mouse homology arm; the final clone was selected based on CM/Hyg resistance.

MAID20433 targeting vector was electroporated into mouse embryonic stem (ES) cells. Targeted homologous recombination resulted in deletion of ˜7 kb of mouse sequence (GRCm38 coordinates chr8: 75093750-75100019). Successful integration was confirmed by a modification of allele (MOA) assay as described, e.g., in Valenzuela et al, supra. Primers and probes used for the MOA assay for the loss of mouse Hmox-1 sequences are depicted in Table 2 below. The cassette was subsequently removed by expression of CRE recombinase (controlled by Protamine promoter) in mice.

TABLE 2

| | | Coordinates |
|---|---|---|
| Probe | | (GRCm38 |
| Name | Probe (5′ to 3′) | assembly) |
| HMOX-1_ | TCAGACGATTTGTAAGATGCAGGGA | chr8: |
| U | (SEQ ID NO. 10) | 75094080- |
| | | 75094104 |
| HMOX-1_ | TGTAGCAGATCCTGGCCTTGGAC | chr8: |
| D | (SEQ ID NO. 11) | 75099760- |
| | | 75099782 |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference). The mouse embryo comprising the donor ES cells was incubated in vitro and then implanted into a surrogate mother to produce an F0 mouse fully derived from the donor ES cells. Mice with Hmox-1 gene deleted were identified by genotyping using the MOA assay described above. Mice heterozygous for deletion of Hmox-1 gene were bred to homozygosity. HMOX-1 KO mice also comprising Rag2 gene knock-out, Il2rg gene knock-out, human M-CSF, humanized Sirpa, and humanized CD47 gene (described in WO 2011/044050, WO 2012/112544, WO 2014/039782, WO 2014/071397, U.S. Pat. No. 11,019,810, WO 2014/039782, WO 2014/071397, WO 2016/168212, U.S. Pat. No. 10,918,095, WO 2018/177440, and WO 2016/089692, each of which is incorporated by reference herein) were obtained either through ES cell modification or breeding. Mice were bred to homozygosity for all genes. These mice are designated as HMOX-1^{-/-} HIS or HMOX-1^{-/-} MSRG47 mice throughout the Examples. Mice were maintained on a sulfa diet (LabDiet, St. Louis, MO) in a MPF facility, and were intra-bred for about 5-10 generations.

### Example 2: Hmox-1 Deletion in Mice Reduces Mouse Macrophages

In order to assess if the deletion of Hmox-1 gene has an effect on mouse macrophages, HMOX-1^{-/-} MSRG47 mouse spleen was harvested into RPMI with 10% fetal bovine serum (FBS), and single cell suspension prepared by mechanical digestion followed by passage through a 70 mm mesh filter. Blood was collected into PBS with 5 mM EDTA via cardiac puncture. Blood and splenic cells were treated with ACK lysing buffer (Gibco) to lyse red blood cells, and cells were then resuspended in PBS with 2% FBS and 1 mM EDTA along with human and mouse Fc block (BD Biosciences). Cells were counted and then stained with anti-mouse CD45 APC-Cy7 (clone 30-F11; BD Biosciences), CD14 FITC (clone Sa14-2; Biolegend), CD11b BV605 (clone M1/70; Biolegend), F4/80 PE-Dazzle (clone BM8; Biolegend), VCAM PE (clone 429; Biolegend), and anti-human CD45 PE-Cy5.5 (clone HI30; Invitrogen). Cells were washed 3× and then acquired on a BD FACS symphony. Analysis was done with FlowJo 10.7 software and cells were gated on hCD45⁻/mCD45⁺ population and then murine monocytes (CD11b⁺/CD14⁺) analyzed along with murine macrophages (F4/80⁺ of CD11b⁺/CD14⁺ cells).

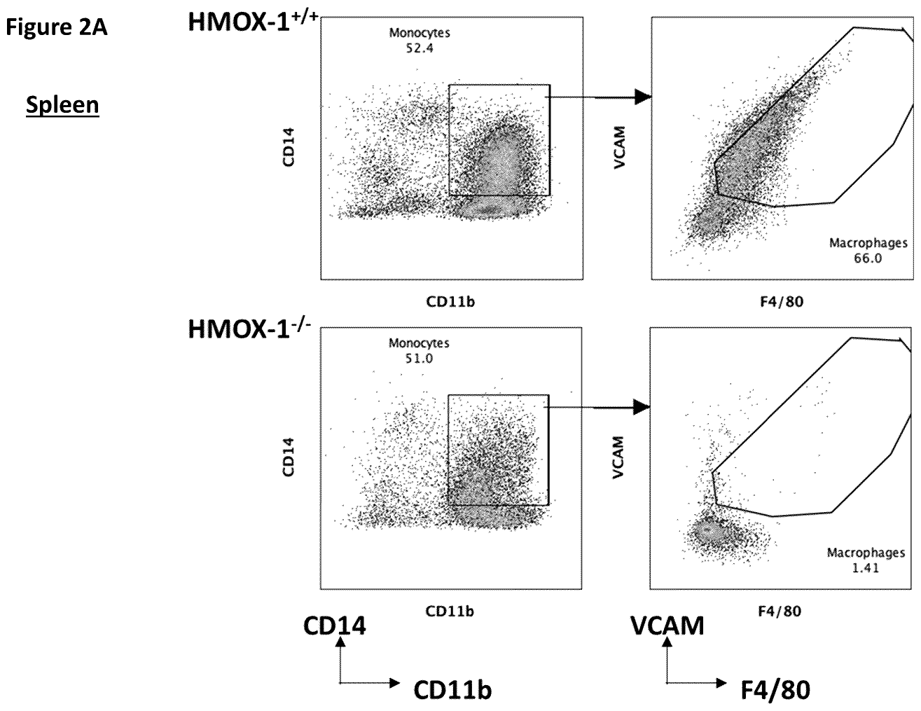

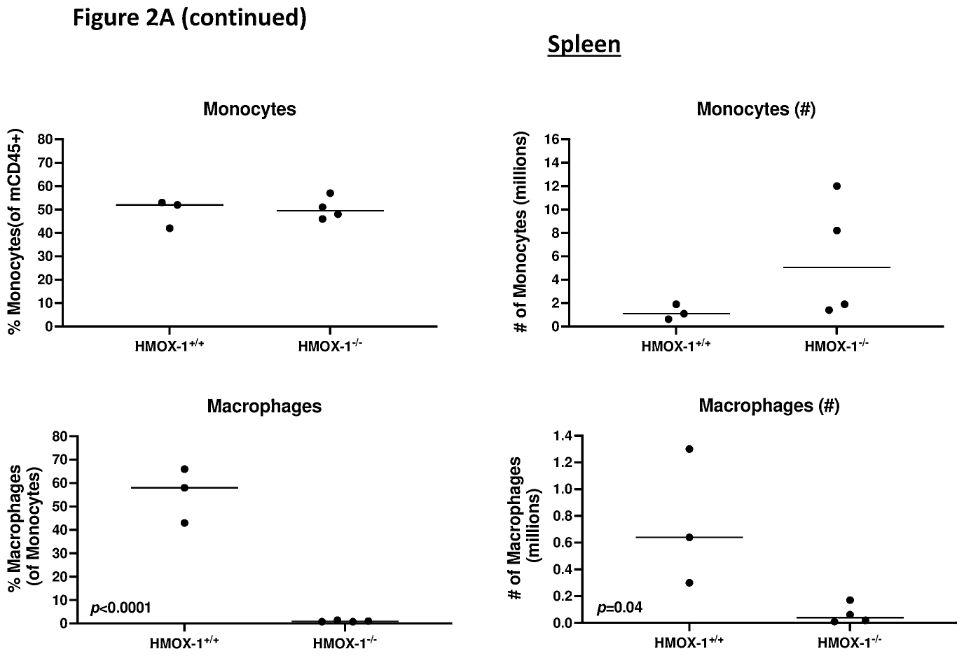

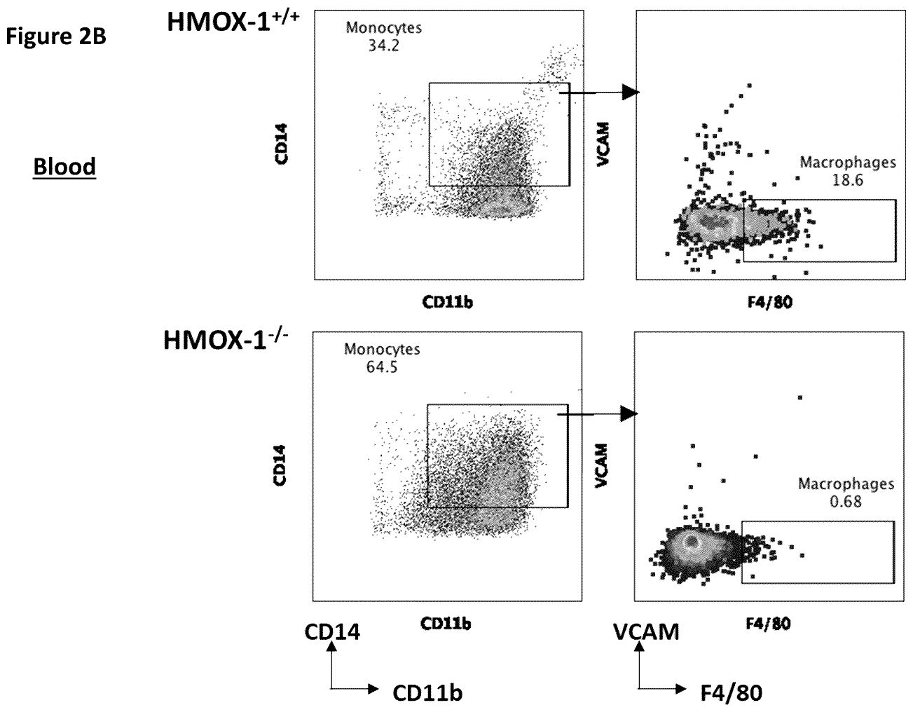

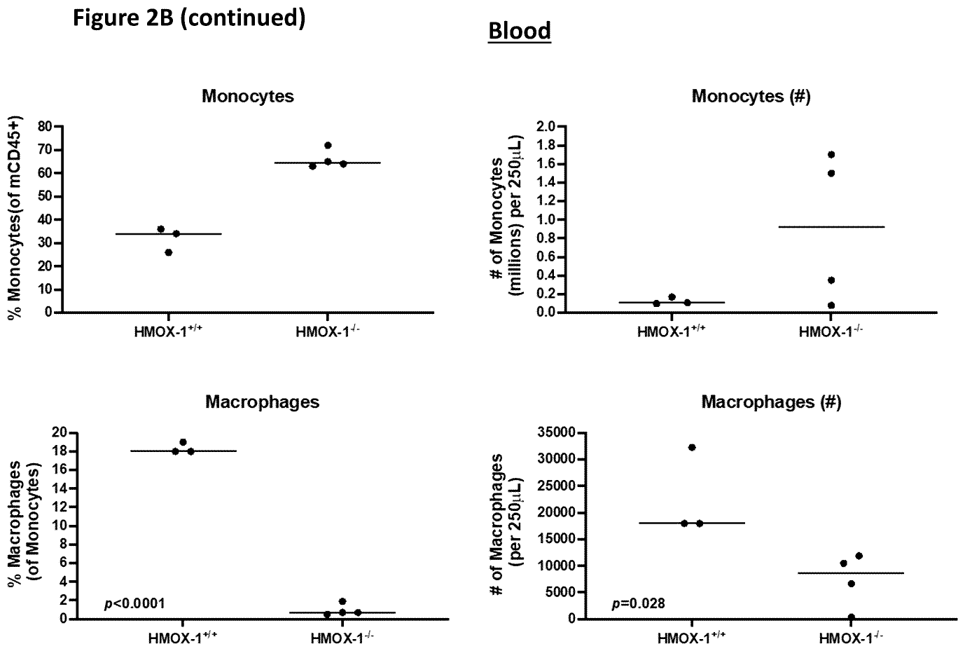

As depicted in FIGS. 2A-B, HMOX-1^{-/-} MSRG47 mice have reduced murine macrophages but no decrease in monocytes in both blood and spleen. Thus, the primary cells responsible for clearance of human red blood cells, murine macrophages, are diminished allowing for enhanced survival of human red blood cells introduced in vivo by hematopoietic stem cell (HSC) engraftment or passive transfusion. Furthermore, by reducing murine macrophages, but still maintaining murine myeloid cells, this mouse strain does not suffer from other pathologies resulting from complete loss of myeloid cells (e.g., murine CSF-1^{-/-}; Dai et al. 2002. Blood. 99: 111-120).

### Example 3: Effect of Hmox-1 Deletion on Human Red Blood Cells

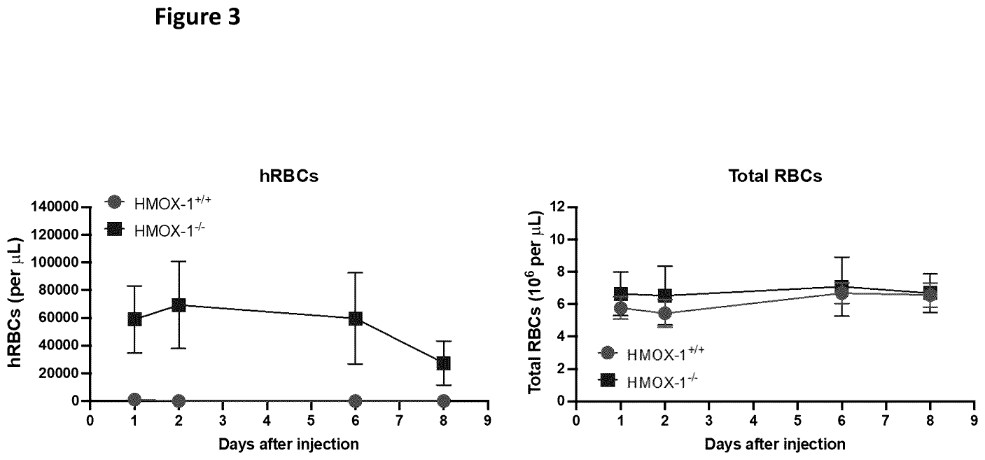

In order to assess the effect of Hmox-1 deletion on human red blood cells, first human RBCs were prepared from human buffy coats (BioIVT) diluted by 2-fold in PBS with 5 mM EDTA. 400 million RBCs were injected intraperitoneally into non-engrafted HMOX-1^{+/+} and HMOX-1^{-/-} MSRG47 mice. Mice were bled retro-orbitally at days 1, 2, 6, and 8 post-injection, and peripheral blood was collected in PBS with 5 mM EDTA and analyzed by FACS staining with anti-human CD235a (marker for human RBCs; clone HI264, Biolegend) and anti-mouse Ter119 PE-Cy7 (marker for murine RBCs; clone TER-119; Biolegend). Cells were acquired on a BD FACS Symphony, and analysis was done with FlowJo 10.7 software gating for human RBCs on CD235a⁺/Ter119⁻ population. As depicted in FIG. 3 (left graph), at day 1 post-injection, no hRBCs could be detected in the peripheral blood of HMOX-1^{+/+} mice, as is expected since it is reported that injected hRBCs are cleared within hours from the peripheral blood of mice. In contrast, injected human red blood cells survived longer (e.g., for more than 1 week in the peripheral blood) in HMOX-1 KO mice relative to mice expressing wild type mouse HMOX-1 gene (FIG. 3 (left graph)). Notably, the level of total RBCs did not change nor was there a difference between HMOX-1^{-/-} and HMOX-1^{+/+} mice (FIG. 3 (right graph)).

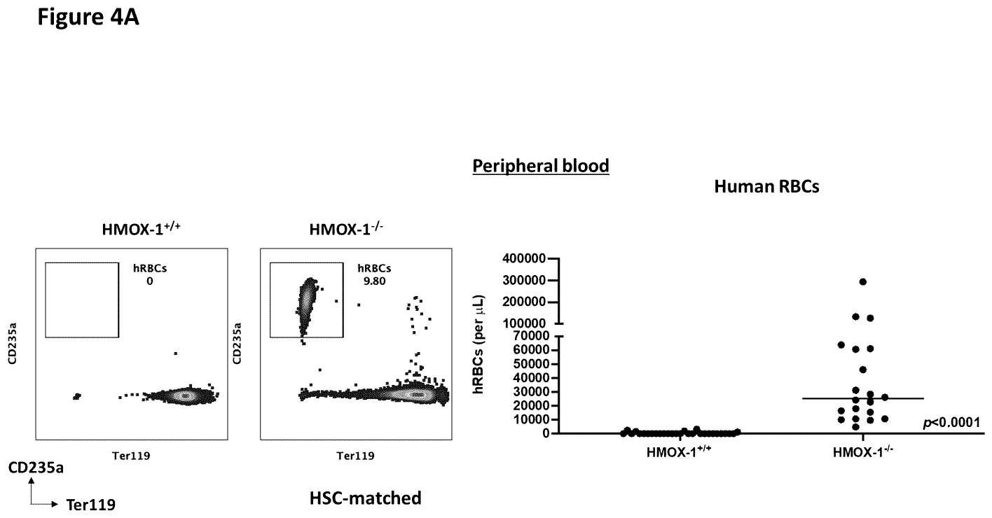

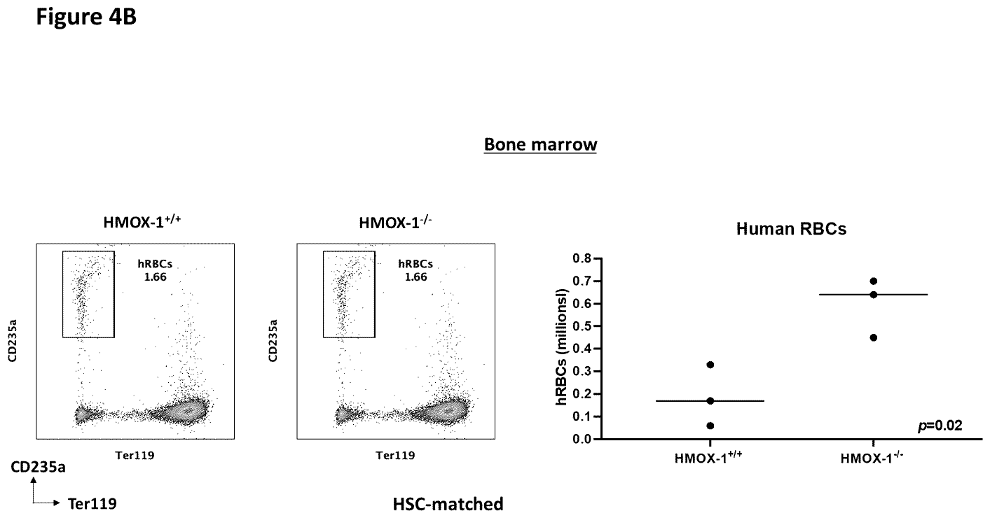

Subsequently, the effect of HMOX-1 deletion on development and survival of human red blood cells in mice engrafted with human hematopoietic stem cells (HSCs) was assessed. Human fetal liver (FL) samples were obtained from Advanced Biosciences Resources (Alameda, CA) with proper consent. FL samples were cut in small fragments, treated for 25 min at 37° C. with Collagenase D (100 ng/mL; Roche). Cell suspension was prepared and the human CD34+ cells were separated by density gradient centrifugation, followed by positive immunomagnetic selection using anti-human CD34 microbeads according to the manufacturer's instructions (Miltenyi Biotec). Cells were either frozen in 10% DMSO containing human albumin serum and kept in liquid nitrogen or injected directly. Newborn pups of HMOX-1^{+/+} and HMOX-1^{-/-} MSRG47 mice were irradiated (160cGy) and injected intrahepatically with 100,000 human hematopoietic stem cells (HSCs) isolated from fetal liver. 12 weeks later mice were retro-orbitally bled and total RBCs counted. Levels of hCD45+ engraftment were analyzed by FACS staining. RBCs were FACS stained with anti-human CD235a PE (marker for human RBCs; clone HI264, Biolegend) and anti-mouse Ter119 PE-Cy7 (marker for murine RBCs; clone TER-119; Biolegend). For overall hematopoietic engraftment, blood was collected retro-orbitally 10 to 12 weeks after engraftment. Red blood cells were lysed using ACK (Gibco) and the cells were stained with the following monoclonal antibodies for flow cytometry analysis: anti-mouse CD45-APC-Cy7 (clone: 30-F11; BD Biosciences) and anti-human CD45-PE-Cy5.5 (clone: HI30; Invitrogen). Cells were acquired on a BD FACS Fortessa or symphony, and analysis was done with FACSDiva (BD Biosciences) and FlowJo 10.7 software gating for human RBCs on CD235a⁺/Ter119⁻ population. Mice with ≥10% hCD45+ of total circulating CD45+ cells (total including both mouse and human CD45+ cells) were used for experiments. For experimental repeats, different donor sources of human CD34+ cells were used. Donor to donor variations were comparable with the range of variation between individual same donor CD34+ cell engrafted mice. As depicted in FIG. 4A, HSC-engrafted HMOX-1 KO mice exhibit human red blood cells in peripheral blood, as compared to their wild type littermates. Graph represents data from 3 different HSC donors. In FIG. 4B, hRBCs were detected in the bone marrow (BM) of both HSC-engrafted MSRG47 HMOX-1^{-/+} and HMOX-1^{-/-} (albeit higher levels in HMOX-1^{-/-}), but as previously reported with HIS mouse models and as confirmed by FIG. 4A, hRBC progenitors developing in the BM could not survive in the peripheral blood in HMOX-1^{+/+} mice.

### Example 4: Effect of Human EPO on Red Blood Cell Levels

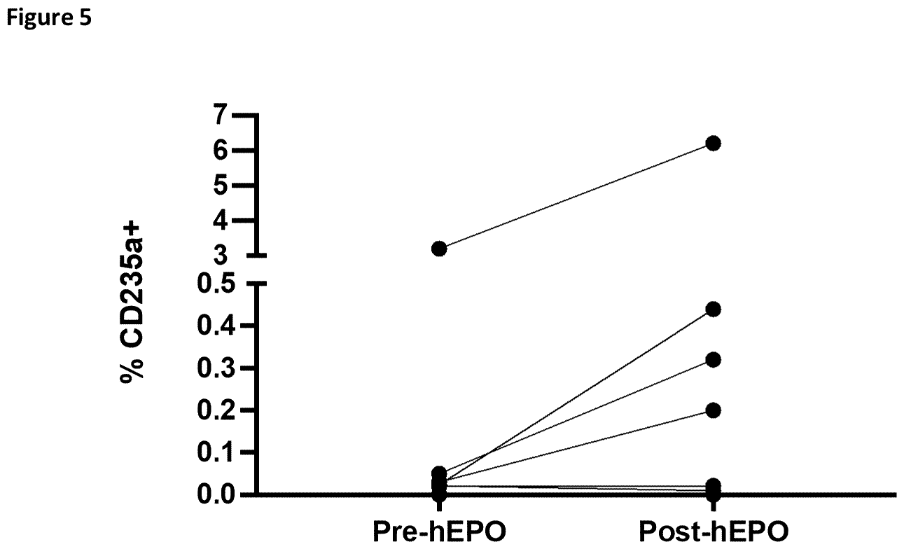

It is known that erythropoietin (EPO) stimulates red blood cell production from the bone marrow. Therefore, it was determined whether injection of human EPO would increase human red blood cells further upon deletion of HMOX-1 gene. To do so, human HSC-engrafted HMOX-1^{-/-} MSRG47 mice were analyzed for pre-injection levels of human RBCs by FACS staining with anti-human CD235a PE (marker for human RBCs; clone HI264, Biolegend) and anti-mouse Ter119 PE-Cy7 (marker for murine RBCs; clone TER-119; Biolegend) as previously described. Mice were injected intra-peritoneally with 100 units of recombinant human erythropoietin (EPO; R&D systems) in PBS. One week post-injection, mice were rebled to check human RBCs level via anti-human CD235a PE (marker for human RBCs; clone HI264, Biolegend) and mouse Ter119 PE-Cy7 (marker for murine RBCs; clone TER-119; Biolegend) FACS staining. As depicted in FIG. 5, injection of human EPO increased human red blood cell levels in peripheral blood of HMOX-1 KO HIS mice.

### Example 5: Generation of HMOX-1 Knock Out Mouse with Humanized EPO

Due to the effect of human EPO on human red blood cell levels as demonstrated in Example 4, Hmox-1 deletion is made onto a HIS mouse strain with humanized EPO. The HMOX-1 KO HIS strain generated in Example 1 is bred to the HIS strains with humanized EPO (such as those described in the international publication No. WO 2015/179317 A2, which is incorporated by reference herein in its entirety). Levels of human red blood cells are compared between HSC-engrafted HMOX-1 KO MSRG47 mice and the same HSC donor-engrafted HMOX-1 KO MSRG47 mice with humanized EPO.

### Example 6: Healthy Kidney/Liver Function in HMOX-1^{-/-} HIS Mouse Model

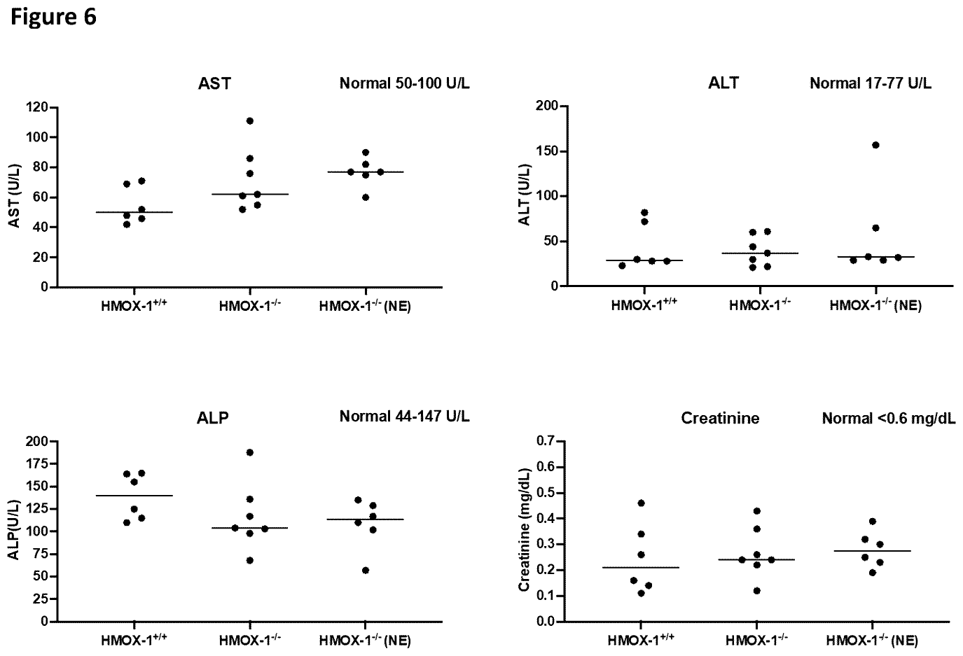

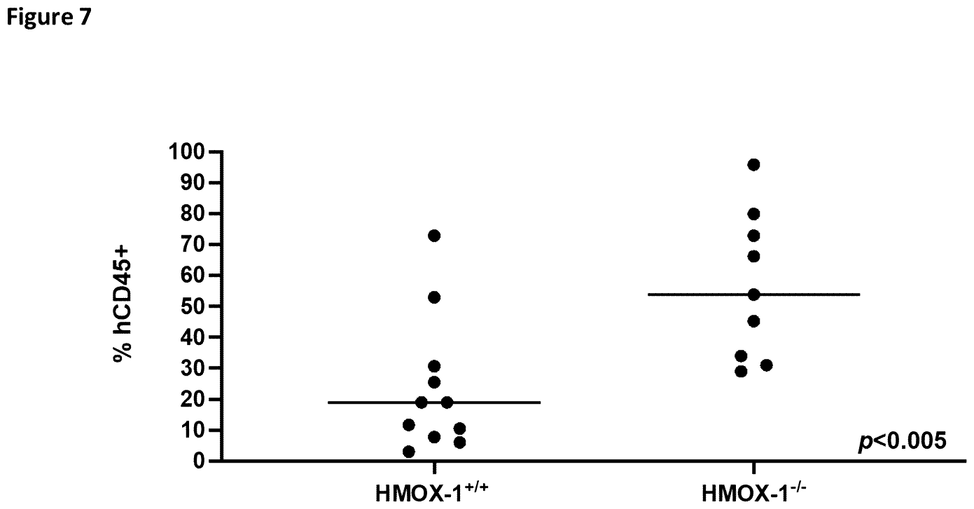

In FIG. 6, serum from HSC-engrafted MSRG47 HMOX-1^{+/+} and MSRG47 HMOX-1^{-/-} was collected along with serum from non-engrafted MSRG47 HMOX-1^{-/-} mice to analyze aspartate aminotransferase (AST), alanine aminotransferase (ALT), creatinine, and alkaline phosphatase (ALP). Elevated levels of these factors are indicative of kidney or liver damage. All mice showed generally normal serum levels of AST, ALT, creatinine, and ALP, which indicated that HMOX-1^{-/-} HIS mice (whether engrafted or non-engrafted) did not have any kidney/liver pathology, in contrast to reports in literature in which the Hmox-1 deletion was on an immune-competent background.

### Example 7: Single Cell RNAseq of HMOX-1^{-/-} HIS Mouse

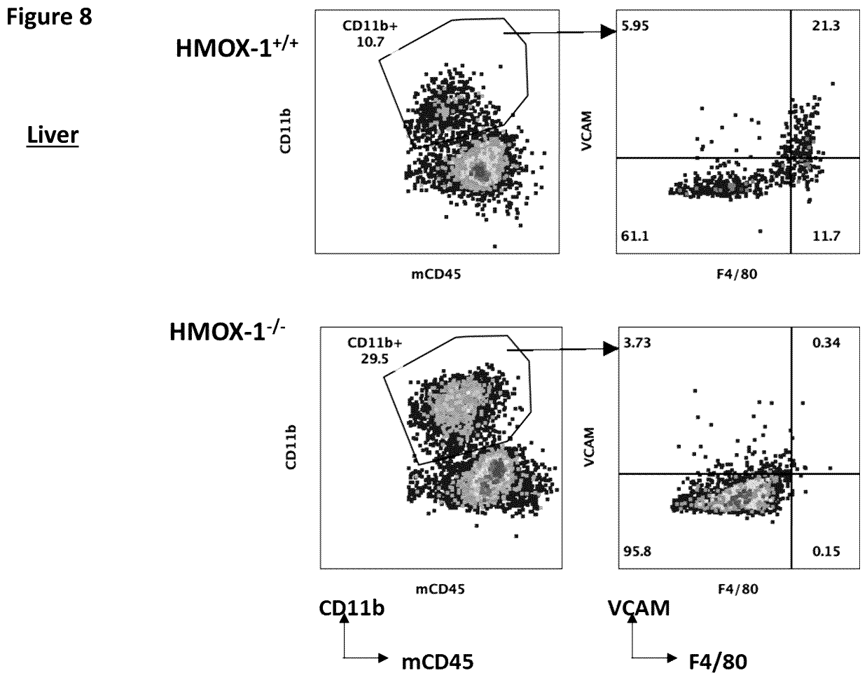

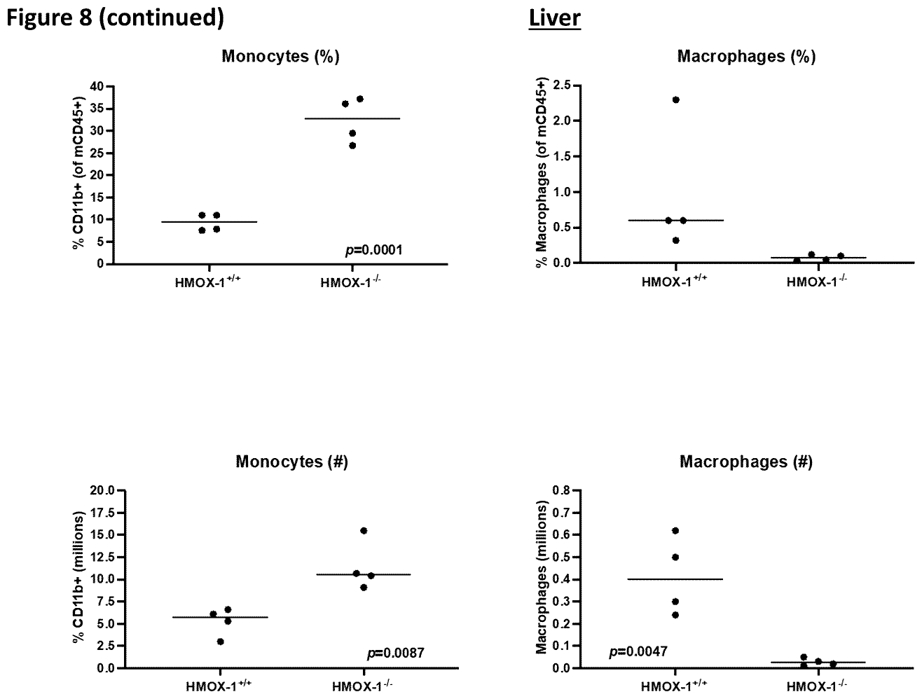

Flow cytometric analysis has already shown a reduction in murine macrophages without a loss of overall murine monocytes in HMOX-1^{-/-} mice (FIGS. 2A, 2B and 8). To further determine if there are differences in human leukocytes that develop from hHSC engraftment of HMOX-1^{+/+} versus HMOX-1^{-/-} HIS mice, single cell RNAseq was performed on mouse and human CD45+ leukocytes isolated from the spleen/BM of HMOX-1^{+/+} and HMOX-1^{-/-} MSRG47 mice. Cells were isolated by positive magnetic enrichment using either mouse CD45 or human CD45 microbeads (Miltenyi). Isolated cells were processed using 10×5prime NextGEM v2 library kit. After the processing, CellRanger (v.6.1.1, 10× Genomics) with GRCh38 reference was performed on the processed cells to compute gene expression levels and to generate analysis-ready data. The quality of the data was checked (number of cells, mean reads per cell, median genes per cell, median UMI counts per cell, etc.) to filter out artifacts, empty droplets, and multiplets. In each sample, cells with the total number of molecules detected within a cell higher than 30,000 and lower than 1,000 were filtered out. Then Python module Scanpy (v1.7.1) was used to normalize the data. The normalization was done as follows: 1. Transformation of total counts so each cell sums to 10,000; 2. Log 1p of the transformed counts; 3. Unit Scaling of the gene expression counts across all cells to ensure a mean of zero and a max value of 10. Lastly, R Seurat package (v4.3.0.1) was utilized to generate UMAP plots and to run clustering. In each cluster, markers were identified based on differentially expressed genes, and cell types were given to the clusters based on the top markers (data not shown). Cell type percentages were calculated by [the number of cells for the cell type×100/the total number of cells in the condition].

The percentage of various subsets of murine CD45+ leukocytes in the spleen of engrafted HMOX-1^{+/+} and HMOX-1^{-/-} MSRG47 mice are shown in Table 5A below.

TABLE 5A
Single cell RNAseq analysis reveals loss of murine erythroblastic
macrophages in spleen of HMOX-1^{-/-} HIS mice

| Cell % | KO | WT |
|---|---|---|
| EB Macrophages | 0.103 | 20.7 |
| Monocytes | 28.26 | 19.23 |
| Neutrophils | 39.27 | 42.96 |
| Eosinophils | 17.96 | 6.978 |
| cDCs | 12.22 | 7.96 |
| pDCs | 2.193 | 2.167 |

* Cell % = cell # of a cell type × 100/all the cell # in the condition

Murine macrophages are selectively diminished in the spleen of HMOX-1^{-/-} mice and these macrophages are denoted “erythroblastic” macrophages (EB macrophages) because they share genes that are commonly found on erythrocytes such as EPOR, KLF1, and Hemoglobin-associated genes Hbb and Hba. This subset of macrophages is involved in the formation of erythroblastic islands for RBC maturation as well as erythrophagocytosis of senescent RBCs (Romano, L. et al. Blood 140, 1621-1634 (2022); Li, W. et al. Blood 134, 480-491 (2019); Li, W. et al. Front Cell Dev Biol 8, 613885 (2020); An, X. et al. Int J Hematol 93, 139-143 (2011)). Remarkably, single cell RNAseq analysis of human CD45+ cells in the spleen indicated a loss of human EB macrophages as well as a significant reduction in human B cells (Table 5B).

TABLE 5B
Single cell RNAseq analysis reveals loss of human erythroblastic
macrophages in spleen of HMOX-1^{-/-} HIS mice

| Cell % | KO | WT |
|---|---|---|
| EB Macrophages | 0.098 | 11.84 |
| Neutrophils | 5.435 | 8.61 |
| B cells | 4.879 | 38.5 |
| Macrophages | 29.24 | 19.31 |
| T cells | 57.3 | 17.48 |
| cDCs | 2.227 | 2.994 |
| pDCs | 0.819 | 1.276 |

* Cell % = cell # of a cell type × 100/all the cell # in the condition

### Example 8: Combining HMOX-1-Deficient HIS Models with Modifications that Allow Human Liver Engraftment

The presence of human RBC in the peripheral blood of HSC-engrafted HMOX-1^{-/-} HIS mouse models offers a unique opportunity for modeling malaria with in vivo mouse models, especially when combined with humanized liver models. Various humanized liver rodent models are known in the art, and described herein above. In this example, FSRG mice (mice comprising a knock-out of Fah gene (FAH^{-/-}), Rag^{-/-}, Il2rg^{-/-}, and humanized Sipra), generated as described in Carbonaro et al. (2023) Sci. Adv. 9, eadf4490 (incorporated herein in its entirety by reference) are bred to HMOX-1^{-/-} MSRG47 mice described herein above. Mice are bred to homozygosity for all genes. Animals are maintained with drinking water containing 2-(2-nitro-4-trifluoromethylbenzoyl)-1,3-cyclohexanedione (NTBC) as described previously (see Carbonaro et al. (2023) supra, and Carbonaro et al. (2022) Sci. Rep. 12:14079; incorporated herein by reference in their entireties).

Newborn pups are irradiated and injected intrahepatically with 100,000 human CD34+ HSCs as described in Example 3 above. After 12 weeks, mice are checked for engraftment by retro-orbital bleed, and presence of human red blood cells is confirmed. Upon engraftment check, mice are subjected to human hepatocyte transplantation intrasplenically after NTBC withdrawal as described in Carbonaro et al. (2022) and (2023), supra. Mice are checked for human liver and human red blood cell engraftment.

Successfully engrafted mice are infected with malaria (P. falciparum) parasite as described in, e.g., Vaughan et al. (2012) J Clin Invest 122(10):3618-28, and both liver and blood stages of malaria infection are studied.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov. A person of ordinary skill in the art would understand that the sequence databases disclosed above or those known in the art are periodically updated to publish corrected sequences. Incorporated by reference in their entirety are such corrected sequences.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1.-110. (canceled)

111. A genetically modified non-human animal cell, comprising: (i) a homozygous null mutation in Rag2 gene; (ii) a homozygous null mutation in IL2rg gene; and (iii) a homozygous null mutation in the non-human animal Heme oxygenase-1 (Hmox-1) gene.

112. The genetically modified non-human animal cell of claim 111, comprising a homozygous null mutation in Rag1 gene.

113. The genetically modified non-human animal cell of claim 111, comprising a homozygous null mutation in Fah gene.

114. The genetically modified non-human animal cell of claim 111, wherein the genetically modified non-human animal cell expresses a human or humanized SIRPA polypeptide encoded by a nucleic acid operably linked to a Sirpa promoter.

115. The genetically modified non-human animal cell of claim 114, wherein the genetically modified non-human animal cell further expresses one or more human or humanized proteins selected from the group consisting of:
a human TPO protein encoded by a nucleic acid operably linked to a TPO promoter;
a human GM-CSF protein encoded by a nucleic acid operably linked to a GM-CSF promoter;
a human IL3 protein encoded by a nucleic acid operably linked to a IL3 promoter;
a human IL15 protein encoded by a nucleic acid operably linked to a IL15 promoter;
a human M-CSF protein encoded by a nucleic acid operably linked to an M-CSF promoter;
a human or humanized CD47 protein encoded by a nucleic acid operably linked to a CD47 promoter; and
a human EPO protein encoded by a nucleic acid operably linked to an EPO promoter.

116. The genetically modified non-human animal cell of claim 111, wherein the genetically modified non-human animal cell is a rodent cell.

117. The genetically modified non-human animal cell of claim 116, wherein the rodent cell is a rat cell or a mouse cell.

118. The genetically modified non-human animal cell of claim 111, wherein the genetically modified non-human animal cell is a non-human animal embryonic stem (ES) cell.

119. A genetically modified non-human animal, comprising: (i) a homozygous null mutation in Rag2 gene knock-out; (ii) a homozygous null mutation in IL2rg gene knock-out; and (iii) a homozygous null mutation in the non-human animal Heme oxygenase-1 (Hmox-1) gene.

120. A method for identifying an agent that inhibits an infection by a pathogen that targets human cells of the erythroid lineage, the method comprising:
a. administering an agent to a genetically modified non-human animal, wherein the genetically modified non-human animal comprises:
i. a homozygous null mutation in the non-human animal Hmox-1 gene;
ii. a homozygous null mutation in Rag2 gene and a homozygous null mutation in IL2rg gene; and
iii. an engraftment of human hematopoietic cells; and
b. (i) infecting the genetically modified non-human animal with a pathogen that targets human cells of the erythroid lineage, and (ii) determining whether the agent reduces the amount of the pathogen and/or inhibits the activity of the pathogen in the pathogen-infected non-human animal;
or
(i) injecting the genetically modified non-human animal with parasitized reticulocytes or erythrocytes, and (ii) determining whether the agent prevents the infection of the human reticulocytes and/or erythrocytes of the non-human animal.

121. The method of claim 120, wherein the pathogen (1) can cause malaria in human, or (2) is selected from a Plasmodium sp., Babesia sp., and a Theileri sp.

122. A method for identifying an agent that treats sickle cell disease, the method comprising:
a. administering the agent to a genetically modified non-human animal, wherein the genetically modified non-human animal comprises:
i. a homozygous null mutation in the non-human animal Hmox-1 gene;
ii. a homozygous null mutation in Rag2 gene and a homozygous null mutation in IL2rg gene; and
iii. an engraftment of human hematopoietic cells comprising a mutation in β-globin gene that leads to sickle cell disease, and
b. determining whether the agent prevents or reduces red cell sickling in the non-human animal.

123. A method for assessing therapeutic efficacy of a drug candidate targeting human red blood cells, the method comprising:
a. administering the drug candidate to a genetically modified non-human animal, wherein the genetically modified non-human animal comprises:
i. a homozygous null mutation in the non-human animal Hmox-1 gene;
ii. a homozygous null mutation in Rag2 gene and a homozygous null mutation in IL2rg gene; and
iii. an engraftment of human hematopoietic progenitor cells, and
b. monitoring the human red blood cells in the non-human animal to assess the therapeutic efficacy of the drug candidate.

124. The method of claim 123, wherein the human red blood cells are monitored to determine whether generation and/or survival of the human red blood cells in the non-human animal is increased by the drug candidate.

125. A method of assessing toxicity of a drug candidate on human red blood cells, comprising:
a. administering the drug candidate to a genetically modified non-human animal, wherein the genetically modified non-human animal comprises:
i. a homozygous null mutation in the non-human animal Hmox-1 gene;
ii. a homozygous null mutation in Rag2 gene and a homozygous null mutation in IL2rg gene; and
iii. an engraftment of human hematopoietic progenitor cells, and
b. monitoring the human red blood cells in the non-human animal to assess the toxicity of the drug candidate.

126. The method of claim 125, wherein the human red blood cells are monitored to (1) determine whether number of the human red blood cells in the non-human animal is reduced by the drug candidate; or (2) assess whether the drug candidate induces agglutination of the red blood cells.

127. The method of claim 126, wherein the drug candidate is a chemotherapeutic agent, an anti-malaria agent, or a modulator of a human CD47 protein.

128. A method of identifying an agent that reduces toxicity of a toxic drug on human red blood cells, comprising:
a. administering the agent and the toxic drug to a genetically modified non-human animal, wherein the genetically modified non-human animal comprises:
i. a homozygous null mutation in the non-human animal Hmox-1 gene;
ii. a homozygous null mutation in Rag2 gene and a homozygous null mutation in IL2rg gene; and
iii. an engraftment of human hematopoietic progenitor cells, and
b. determining whether the agent reduces the toxicity of the toxic drug on human red blood cells in the non-human animal.

129. The method of claim 128, wherein the agent and the toxic drug are administered to the non-human animal concurrently or sequentially.

130. A method of making a non-human animal embryonic stem cell, comprising genetically engineering the non-human animal embryonic stem cell so that the non-human animal embryonic stem cell has a genome that comprises: (i) a homozygous null mutation in Rag2 gene; (ii) a homozygous null mutation in IL2rg gene; and (iii) a homozygous null mutation in the non-human animal Heme oxygenase-1 (Hmox-1) gene.

131. A non-human animal embryo comprises the non-human animal embryonic stem cell of claim 118.

132. A method of making a non-human animal comprising in its genome: (i) a Rag2 gene knock-out; (ii) a IL2rg gene knock-out; and (iii) a homozygous null mutation in the non-human animal Heme oxygenase-1 (Hmox-1) gene at the non-human animal Hmox-1 gene locus, the method comprising steps of:
(a) obtaining a non-human animal embryonic stem cell of claim 118; and
(b) creating a non-human animal using the embryonic cell of (a).

133. A method of making a non-human animal comprising in its genome: (i) a Rag2 gene knock-out; (ii) a IL2rg gene knock-out; and (iii) a homozygous null mutation in the non-human animal Heme oxygenase-1 (Hmox-1) gene, the method comprising modifying the genome of the non-human animal so that it comprises: (i) a Rag2 gene knock-out; (ii) a IL2rg gene knock-out; and (iii) a homozygous null mutation in the non-human animal Heme oxygenase-1 (Hmox-1) gene at the non-human animal Hmox-1 gene locus.
